(19) United States
(12) Patent Application Publication
(10) Pub. No.: US 2011/0177070 A1
(43) Pub. Date: Jul. 21, 2011

(54) TGF-Beta Antagonist Multi-Target Binding Proteins

(75) Inventors: Alan Keith Lofquist, Kirkland, WA (US); Peter Robert Baum, Seattle, WA (US); Peter Armstrong Thompson, Bellevue, WA (US); Lynda Misher, Seattle, WA (US)

(73) Assignee: Emergent Product Development Seatlle, LLC

(21) Appl. No.: 13/001,088

(22) PCT Filed: Jul. 2, 2009
(86) PCT No.: PCT/US2009/049616

§ 371 Date: Mar. 3, 2011

Related U.S. Application Data
(60) Provisional application No. 61/134,091, filed on Jul. 2, 2008.
(60) Provisional application No. 61/134,090, filed on Jul. 2, 2008.
(60) Provisional application No. 61/134,089, filed on Jul. 2, 2008.
(60) Provisional application No. 61/134,088, filed on Jul. 2, 2008.
(60) Provisional application No. 61/134,085, filed on Jul. 2, 2008.
(60) Provisional application No. 61/134,087, filed on Jul. 2, 2008.
(60) Provisional application No. 61/134,086, filed on Jul. 2, 2008.
(60) Provisional application No. 61/181,232, filed on May 26, 2009.

(51) Int. Cl. A61K 39/395; C07K 16/46; C07H 21/04; C12N 15/63; C12N 5/10; A61P 35/00; C12P 21/04; A61P 19/02

(52) U.S. Cl. 424/134.1; 530/387.3; 536/23.4; 435/320.1; 435/369; 435/69.6

(57) ABSTRACT

This disclosure provides a multi-target fusion protein composed of a TGF? antagonist domain and another binding domain antagonistic for a heterologous target (such as IL6, IL10, VEGF, TNF, HGF, TWEAK, IGF) or agonistic for a heterologous target (such as GITR). The multi-specific fusion protein may also include an intervening domain that separates the binding domains and allows for dimerization. This disclosure also provides polynucleotides encoding the multi-specific fusion proteins, compositions of the fusion proteins, and methods of using the multi-specific fusion proteins and compositions.

### TECHNICAL FIELD

This disclosure relates generally to the field of multi-target binding molecules and therapeutic applications thereof and more specifically to a fusion protein composed of either a transforming growth factor-beta (TGFβ) antagonist domain and another binding domain antagonistic for a heterologous target, such as IL6, IL10, VEGF, TNF, HGF, TWEAK, IGF1 or IGF2, or a TGFβ antagonist domain and another binding domain agonistic for a heterologous target, such as GITR, as well as compositions and therapeutic uses thereof.

### BACKGROUND

Transforming growth factor-beta (TGFβ) is a potent cytokine that has significant effects on the immune system. The main function of TGFβ in the immune system is to maintain tolerance and initial immune responses against foreign pathogens. Three isoforms of TGFβ have been identified in mammals, TGFβ1, TGFβ2 and TGFβ3, with TGFβ1 being the predominant isoform. TGFβ is secreted in a latent form and only a small percentage of total secreted TGFβ is activated under physiological conditions. The biological effects of TGFβ occur mostly through binding of TGFβ to the receptors ALK5 and TGFβ receptor II (TGFβR2). Specifically, active TGFβ dimer binds to a tetrameric ALK5 and TGFβR2 complex to initiate cell signaling. ALK5 is not required for the initial binding of TGFβ, but is required for signaling.

TGFβ has been shown to influence many cellular functions such as cell proliferation, differentiation, cell-cell and cell-matrix adhesion, cell motility and activation of lymphocytes. (For a review of the role of TGFβ in regulating immune responses, see Li et al. (2006) Annu Rev. Immunol. 24:99-146.) Furthermore, TGFβ is believed to induce or mediate the progression of many diseases such as osteoporosis, hypertension, atherosclerosis, hepatic cirrhosis and fibrotic diseases of the kidney, liver and lungs, and tumor progression. TGFβ can augment end-organ damage caused by chronic inflammation and TGFβ antagonists have been shown to be effective in attenuating this damage in animal models of diseases such as diabetic kidney disease, glomerulonephritis, cyclosporine-mediated renal injury and systemic lupus erythematosus (SLE) (Border et al. (1990) Nature 346:371-374; Border et al. (1992) Nature 360:361-364; Isaka et al. (1999) Kidney Int. 55:465-475; Sharma et al. (1996) Diabetes 45:522; Xin et al. (2004) Transplantation 15:1433; Benigni et al. (2003) J. Am. Soc. Nephrol. 14:1816). With respect to cancer, TGFβ can have a direct inhibitory activity on malignant cells and can augment the production or activity of a range of tumor growth factors and angiogenic factors.

While TGFβ knock-out mice have severe pathology related to unrestrained inflammation and autoimmunity, administration of TGFβ antagonists is well tolerated in mice and humans (Rusek et al. (2003) Immunopharmacol. Immunotoxicol. 25:235-57; Denton et al. (2007) Arthritis Rheum. 56:323-33). Methods of treatment using TGF antagonists known in the art include use of antibodies against TGFβ, use of TGFβR2 ectodomain Ig fusion proteins, and use of small molecule inhibitors of TGFβ1 kinase activity. All of these methods have modest beneficial impact in rodent models of disease or in clinical trials in humans (Denton et al. (2007) Arthritis Rheum. 56:323). Indeed, chronic use of a TGFβ antagonist in mice shows no evidence of activation of the immune system as might be expected from the phenotype of TGFβ−/− knock-out mice. This is likely to reflect, in part, the complex nature of the biology of cytokines, interleukins, chemokines and growth factors in human diseases and the requirement to inhibit more than one pathway simultaneously to maximize the benefit to patients.

### BRIEF DESCRIPTION OF THE FIGURES

FIGS. 1A-1C show that multi-specific (Xceptor) fusion proteins containing one of various different Hyper-IL6 binding domains fused to a TNFR ectodomain bind to Hyper-IL6 specifically as measured by ELISA, and that these multi-specific fusion proteins preferentially bind Hyper-IL6 over IL6 and IL6R alone. Only two fusion proteins tested bound IL6 and none bound sIL6R.

FIG. 2 shows that multi-specific fusion proteins containing a TNFR ectodomain fused to one of various different Hyper-IL6 binding domains bind to TNF-α as measured by ELISA.

FIG. 3 shows that multi-specific fusion proteins containing one of various different Hyper-IL6 binding domains fused to a TNFR ectodomain can simultaneously bind to Hyper-IL6 and TNF-α as measured by ELISA.

FIG. 4 shows that multi-specific fusion proteins containing one of various different Hyper-IL6 binding domains fused to a TNFR ectodomain block gp130 from binding to Hyper-IL6 as measured by ELISA.

FIGS. 5A and 5B show that multi-specific fusion proteins containing one of various different Hyper-IL6 binding domains fused to a TNFR ectodomain block (A) IL6 or (B) Hyper-IL6 induced proliferation of TF-1 cells.

FIG. 6 shows that multi-specific fusion proteins containing one of various different Hyper-IL6 binding domains fused to a TNFR ectodomain block TNF-α from binding to TNFR as measured by ELISA.

FIG. 7 shows that multi-specific fusion proteins containing a TNFR ectodomain fused to one of various different Hyper-IL6 binding domains block TNF-α induced killing of L929 cells.

FIG. 8 shows that multi-specific fusion proteins containing a TGFβR2 ectodomain fused to one of various different Hyper-IL6 binding domains bind to TGFβ1 as measured by ELISA.

FIG. 9 shows that multi-specific fusion proteins containing a TNFR ectodomain fused to a TGFβRII ectodomain block TGFβ-1 induced inhibition of IL-4 proliferation of HT2 cells.

FIG. 10 shows that multi-specific fusion proteins containing a TNFR ectodomain fused to an IL6 binding domain did not bind to HepG2 (liver) cells.

FIG. 11 shows that multi-specific fusion proteins containing a TNFR ectodomain fused to an IL6 binding domain blocked the HIL6-induced SAA response in mice.

FIG. 12 shows that multi-specific fusion proteins containing a TNFR ectodomain fused to an IL6 binding domain blocked the HIL6-induced sgp130 response in mice.

FIGS. 13A and B show the results of studies on the ability of multi-specific fusion proteins containing a TNFR ectodomain fused to an IL6 binding domain to block the TNFα-induced SAA response in mice, at 2 hours and 24 hours post-administration, respectively.

### DETAILED DESCRIPTION

The present disclosure provides multi-specific fusion proteins, referred to herein as Xceptor molecules. Exemplary structures of such multi-specific fusion proteins, include N-BD-ID-ED-C, N-ED-ID-BD-C, and N-ED1-ID-ED2-C, wherein N- and -C represent the amino- and carboxy-terminus, respectively, BD is an immunoglobulin-like or immunoglobulin variable region binding domain, ID is an intervening domain, and ED is an ectodomain (e.g. an extracellular domain), such as a receptor ligand binding domain, cysteine rich domain (A domain; see WO 02/088171 and WO 04/044011), semaphorin or semaphorin-like domain, or the like. In some constructs, the ID can comprise an immunoglobulin constant region or sub-region disposed between the first and second binding domains. In still further constructs, the BD and ED are each linked to the ID via the same or different linker (e.g., a linker comprising one to 50 amino acids), such as an immunoglobulin hinge region (made up of, for example, the upper and core regions) or functional variant thereof, or a lectin interdomain region or functional variant thereof, or a cluster of differentiation (CD) molecule stalk region or functional variant thereof.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “comprise” are used synonymously. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

A “binding domain” or “binding region” according to the present disclosure may be, for example, any protein, polypeptide, oligopeptide, or peptide that possesses the ability to specifically recognize and bind to a biological molecule (e.g., TGFβ or IL6) or complex of more than one of the same or different molecule or assembly or aggregate, whether stable or transient (e.g., IL6/IL6R complex). Such biological molecules include proteins, polypeptides, oligopeptides, peptides, amino acids, or derivatives thereof, lipids, fatty acids, or derivatives thereof; carbohydrates, saccharides, or derivatives thereof; nucleotides, nucleosides, peptide nucleic acids, nucleic acid molecules, or derivatives thereof; glycoproteins, glycopeptides, glycolipids, lipoproteins, proteolipids, or derivatives thereof; other biological molecules that may be present in, for example, a biological sample; or any combination thereof. A binding region includes any naturally occurring, synthetic, semi-synthetic, or recombinantly produced binding partner for a biological molecule or other target of interest. A variety of assays are known for identifying binding domains of the present disclosure that specifically bind with a particular target, including Western blot, ELISA, or Biacore analysis.

Binding domains and fusion proteins thereof of this disclosure can be capable of binding to a desired degree, including “specifically or selectively binding” a target while not significantly binding other components present in a test sample, if they bind a target molecule with an affinity or Kₐ (i.e., an equilibrium association constant of a particular binding interaction with units of 1/M) of, for example, greater than or equal to about 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173; 5,468,614; Biacore® analysis; or the equivalent).

Binding domains of this disclosure can be generated as described herein or by a variety of methods known in the art (see, e.g., U.S. Pat. Nos. 6,291,161; 6,291,158). Sources include antibody gene sequences from various species (which can be formatted as antibodies, sFvs, scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, ovine, sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., US Patent Application Publication No. 2007/0065431), C-type lectin domains (Zelensky and Gready (2005) FEBS J. 272:6179), mAb² or Fcab™ (see, e.g., PCT Patent Application Publication Nos. WO 2007/098934; WO 2006/072620), or the like. Additionally, traditional strategies for hybridoma development using a synthetic single chain IL6/IL6R complex, such as a human IL6/IL6R complex or Hyper-IL6 (IL6 joined by a peptide linker to IL6R), as an immunogen in convenient systems (e.g., mice, HuMAb Mouse®, TC Mouse™, KM-Mouse®, llamas, chicken, rats, hamsters, rabbits, etc.) can be used to develop binding domains of this disclosure.

Terms understood by those in the art as referring to antibody technology are each given the meaning acquired in the art, unless expressly defined herein. For example, the terms “V_{L}” and “V_{H}” refer to the variable binding region derived from an antibody light and heavy chain, respectively. The variable binding regions are made up of discrete, well-defined sub-regions known as “complementarity determining regions” (CDRs) and “framework regions” (FRs). The terms “C_{L}” and “C_{H}” refer to an “immunoglobulin constant region,” i.e., a constant region derived from an antibody light or heavy chain, respectively, with the latter region understood to be further divisible into C_{H1}, C_{H2}, C_{H3} and C_{H4} constant region domains, depending on the antibody isotype (IgA, IgD, IgE, IgG, IgM) from which the region was derived. A portion of the constant region domains makes up the Fc region (the “fragment crystallizable” region), which contains domains responsible for the effector functions of an immunoglobulin, such as ADCC (antibody-dependent cell-mediated cytotoxicity), ADCP (antibody-dependent cell-mediated phagocytosis), CDC (complement-dependent cytotoxicity) and complement fixation, binding to Fc receptors, greater half-life in vivo relative to a polypeptide lacking an Fc region, protein A binding, and perhaps even placental transfer (see Capon et al. (1989) Nature, 337:525). Further, a polypeptide containing an Fc region allows for dimerization or multimerization of the polypeptide. A “hinge region,” also referred to herein as a “linker,” is an amino acid sequence interposed between and connecting the variable binding and constant regions of a single chain of an antibody, which is known in the art as providing flexibility in the form of a hinge to antibodies or antibody-like molecules.

The domain structure of immunoglobulins is amenable to engineering, in that the antigen binding domains and the domains conferring effector functions may be exchanged between immunoglobulin classes and subclasses. Immunoglobulin structure and function are reviewed, for example, in Harlow et al., Eds., Antibodies: A Laboratory Manual, Chapter 14 (Cold Spring Harbor Laboratory, Cold Spring Harbor, 1988). An extensive introduction as well as detailed information about all aspects of recombinant antibody technology can be found in the textbook Recombinant Antibodies (John Wiley & Sons, NY, 1999). A comprehensive collection of detailed antibody engineering lab Protocols can be found in R. Kontermann and S. Dübel, Eds., The Antibody Engineering Lab Manual (Springer Verlag, Heidelberg/New York, 2000).

“Derivative” as used herein refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, a derivative may be more hydrophilic or it may have altered reactivity (e.g., a CDR having an amino acid change that alters its affinity for a target) as compared to the parent compound.

The term “biological sample” includes a blood sample, biopsy specimen, tissue explant, organ culture, biological fluid or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid sequences, somatic cell hybrid cell lines, immortalized or immortalizable cell lines, differentiated or differentiatable cell lines, transformed cell lines, or the like. In further embodiments of this disclosure, a subject or biological source may be suspected of having or being at risk for having a disease, disorder or condition, including a malignant disease, disorder or condition or a B cell disorder. In certain embodiments, a subject or biological source may be suspected of having or being at risk for having a hyperproliferative, inflammatory, or autoimmune disease, and in certain other embodiments of this disclosure the subject or biological source may be known to be free of a risk or presence of such disease, disorder, or condition.

In certain embodiments, the present disclosure makes possible the depletion or modulation of cells associated with aberrant TGFβ activity by providing multi-specific fusion proteins that bind both a TGFβ and a second target other than TGFβ, such as IL6, IL6R, an IL6/IL6R complex, IL10, GITR, VEGF, TNF, HGF, Tumor necrosis factor-like weak inducer of apoptosis (TWEAK; also known as tumor necrosis factor (ligand) superfamily, member 12, TNFSF12), IGF1 or IGF2. In certain embodiments, a multi-specific fusion protein comprises a first and second binding domain, a first and second linker, and an intervening domain, wherein one end of the intervening domain is fused via a linker to a first binding domain that is a TGFβ2 ectodomain (e.g. an extracellular domain) and at the other end fused via a linker to a second binding domain. In some embodiments, less than an entire TGFβ2 ectodomain is employed. Specifically, domains within the ectodomain that function as a TGFβ antagonist or confer ligand binding are employed.

In certain embodiments, the second binding domain is an IL6 antagonist (such as an immunoglobulin variable region that is specific for an IL6, IL6R, or IL6/IL6Ra complex), an IL10 antagonist (such as an immunoglobulin variable region that is specific for IL10, an IL10R1 ectodomain (e.g. SEQ ID NO:745) or a sub-domain of an IL10R1 ectodomain), a GITR agonist (such as an immunoglobulin variable region that is specific for GITR, a GITRL ectodomain (for example, amino acids 74-181 of Genbank Accession NP_{—}005083.2, SEQ ID NO:746) or a sub-domain of a GITRL ectodomain), a VEGF antagonist (such as an immunoglobulin variable region that is specific for VEGF, a VEGFR2 ectodomain (see, Genbank Accession NP_{—}002244.1, SEQ ID NO:747) or a sub-domain of a VEGFR2 ectodomain), a TNF antagonist (such as an immunoglobulin variable region that is specific for TNF, a TNFR1 ectodomain (see, Genbank Accession NP_{—}001056.1; SEQ ID NO:749), a sub-domain of a TNFR1 ectodomain, a TNFR2 ectodomain (see, Genbank Accession NP_{—}001057.1; SEQ ID NO:748), or a sub-domain of a TNFR2 ectodomain), a HGF antagonist (such as an immunoglobulin variable region that is specific for HGF, a c-Met ectodomain or a sub-domain of a c-Met ectodomain (e.g. SEQ ID NO:750-752)), a TWEAK antagonist (such as an immunoglobulin binding domain specific for TWEAK or TWEAKR, or a TWEAKR ectodomain (e.g. SEQ ID NO:761) or TWEAK binding fragment thereof), or an IGF1 or IGF2 antagonist (such as an immunoglobulin variable region that is specific for IGF1 or IGF2, an IGF1R ectodomain (for example, an IGF1R ectodomain of Genbank Accession no. NP_{—}000866.1 (SEQ ID NO:753) or a sub-domain thereof), or an IGFBP (for example, an IGFBP ectodomain of Genbank Accession no. NP_{—}000587.1 (IGFBP1; SEQ ID NO:754), NP_{—}000588.2 (IGFBP2; SEQ ID NO:755), NP_{—}001013416.1 (IGFBP3 isoform a; SEQ ID NO:756), NP_{—}000589.2 (IGFBP3 isoform b; SEQ ID NO:757), NP_{—}001543.2 (IGFBP4; SEQ ID NO:758), NP_{—}000590.1 (IGFBP5; SEQ ID NO:759) or NP_{—}002169.1 (IGFBP6; SEQ ID NO:760)), or a sub-domain thereof).

The complex of IL6 with membrane or soluble IL6 receptor (IL6Rα) is referred to herein as IL6xR when referring to IL6 with either membrane IL6Rα or soluble IL6Rα (sIL6Rα), and as sIL6xR when referring only to the complex of IL6 with sIL6Rα. In some embodiments, multi-specific fusion proteins containing a binding domain specific for IL6xR have one or more of the following properties: (1) greater or equal affinity for an IL6xR complex than for IL6 or IL6Rα alone or has greater affinity for IL6Rα alone or an IL6xR complex than for IL6 alone; (2) compete with membrane gp130 for binding with a sIL6xR complex or enhance soluble gp130 binding with a sIL6xR complex; (3) preferentially inhibit IL6 trans-signaling over IL6 cis-signaling and (4) do not inhibit signaling of gp130 family cytokines other than IL6.

### TGFβ Antagonists

As outlined above, TGFβ has been linked to several diseases such as fibrosis, auto-immunity and cancer. In the early stages of tumor development, TGFβ acts as a growth inhibitory factor. However, as tumors evolve they develop mechanisms to evade the growth-inhibition properties of TGFβ, resulting in increased tumor invasiveness, increased metastatic potential and inhibition of surrounding immune responses (Luwor et al. (2008) J. Clin. Neurosci. June 10 (epub)). A TGFβ antagonist of this disclosure inhibits the tumor-promoting activity of TGF. The antagonist domains may block TGFβ dimerization and TGFβ binding, or the domains may bind to components of the receptor system and block activity either by preventing ligand activity or by preventing the assembly of the receptor complex.

In some embodiments, a TGFβ antagonist may be an extracellular domain (“ectodomain”) of TGFβR2. In certain embodiments, a TGFβ antagonist comprises a TGFβR2 ectodomain as set forth in SEQ ID NO:743, 744 or any combination thereof.

In one aspect, a TGFβ antagonist or fusion protein thereof of this disclosure is specific for TGFβ wherein it has an affinity with a dissociation constant (K_{d}) of about 10⁻⁵ M to 10⁻¹³ M, or less. In certain embodiments, the TGFβ antagonist or fusion protein thereof binds TGFβ with an affinity that is less than about 300 pM. Another measure, the kinetic dissociation (k_{d}), also referred to herein as k_{OFF}, is a measure of the rate of complex dissociation and, thus, the ‘dwell time’ of the target molecule bound by a polypeptide binding domain of this disclosure. The k_{d} (k_{OFF}) has units of 1/sec. Exemplary TGFβ antagonists of this disclosure can have a k_{OFF} of about 10⁻⁴/sec (e.g., about a day) to about 10⁻⁸/sec or less. In certain embodiments, the k_{OFF} can range from about 10⁻¹/sec, about 10⁻²/sec, about 10⁻³/sec, about 10⁻⁴/sec, about 10⁻⁵/sec, about 10⁻⁶/sec, about 10⁻⁷/sec, about 10⁻⁸/sec, about 10⁻⁹/sec, about 10⁻¹°/sec, or less (see Graff et al. (2004) Protein Eng. Des. Sel. 17:293). In some embodiments, a TGFβ antagonist or fusion protein thereof of this disclosure will bind TGFβ with higher affinity and have a lower k_{OFF} rate as compared to the cognate TGFβ receptor binding to TGFβ. In further embodiments, a TGFβ antagonist or fusion protein thereof of this disclosure that blocks or alters TGFβ dimerization or other cell surface activity may have a more moderate affinity (i.e., a K_{d} of about 10⁻⁸ M to about 10⁻⁹ M) and a more moderate off rate (i.e., a k_{OFF} closer to about 10⁻⁴/sec) as compared to the affinity and dimerization rate of cognate TGFβ receptor.

Exemplary binding domains that function as TGFβ antagonists of this disclosure can be generated as described herein or by a variety of methods known in the art (see, e.g., U.S. Pat. Nos. 6,291,161; 6,291,158). Sources include antibody gene sequences from various species (which can be formatted as scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, ovine, sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., US Patent Application Publication No. 2007/0065431), C-type lectin domains (Zelensky and Gready (2005) FEBS J. 272:6179), or the like. Additionally, traditional strategies for hybridoma development using a synthetic TGFβ or single chain TGFβR2 ectodomain as an immunogen in convenient systems (e.g., mice, HuMAb Mouse®, TC Mouse™, KM-Mouse®, llamas, chicken, rats, hamsters, rabbits, etc.) can be used to develop binding domains of this disclosure.

In an illustrative example, TGFβ antagonists of this disclosure specific for a TGFβ can be identified using a Fab phage library of fragments (see, e.g., Hoet et al. (2005) Nature Biotechnol. 23:344) by screening for binding to a synthetic or recombinant TGFβ (using an amino acid sequence or fragment thereof as set forth in GenBank Accession No. NP_{—}000651.3). A TGFβ, as described herein or known in the art, can be used for such a screening. In certain embodiments, a TGFβ used to generate a TGFβ antagonist can further comprise an intervening domain or a dimerization domain, as described herein, such as an immunoglobulin Fc domain or fragment thereof.

In some embodiments, TGFβ antagonist domains of this disclosure comprise V_{H} and V_{L} domains as described herein. In certain embodiments, the V_{H} and V_{L} domains are rodent (e.g., mouse, rat), humanized, or human. In further embodiments, there are provided TGFβ antagonist domains of this disclosure that have a sequence that is at least 90%, at least 91%, at least 92%, at least 93%, at least 94%, at least 95%, at least 96%, at least 97%, at least 98%, at least 99%, at least 99.5%, or at least 100% identical to the amino acid sequence of one or more light chain variable regions (V_{L}) or to one or more heavy chain variable regions (V_{H}), or both, wherein each CDr has up to three amino acid changes (i.e., many of the changes are in the framework region(s)), as set forth herein.

In further embodiments, TGFβ antagonist domains of this disclosure comprise V_{H} and V_{L} domains as set forth herein, which are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of such V_{H} domain, V_{L} domain, or both wherein each CDr has at most up to three amino acid changes (i.e., many of the changes are in the framework region(s)).

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al. (1977) Nucleic Acids Res. 25:3389 and Altschul et al. (1990) J. Mol. Biol. 215:403, respectively.

In any of these or other embodiments described herein, the V_{L} and V_{H} domains may be arranged in either orientation and may be separated by about a five to about a thirty amino acid linker as disclosed herein or any other amino acid sequence capable of providing a spacer function compatible with interaction of the two sub-binding domains. In certain embodiments, a linker joining the V_{H} and V_{L} domains comprises an amino acid sequence as set forth in SEQ ID NO:497-604 and 1223-1228, such as Linker 47 (SEQ ID NO:543) or Linker 80 (SEQ ID NO:576). Multi-specific binding domains will have at least two specific sub-binding domains, by analogy to camelid antibody organization, or at least four specific sub-binding domains, by analogy to the more conventional mammalian antibody organization of paired V_{H} and V_{L} chains.

In further embodiments, TGFβ antagonist domains and fusion proteins thereof of this disclosure may comprise a binding domain including one or more complementarity determining region (“CDR”), or multiple copies of one or more such CDRs, which have been obtained, derived, or designed from variable regions of an anti-TGFβ or anti-TGFβR2 scFv or Fab fragment or from heavy or light chain variable regions thereof.

CDRs are defined in various ways in the art, including the Kabat, Chothia, AbM, and contact definitions. The Kabat definition is based on sequence variability and is the most commonly used definition to predict CDR regions (Johnson et al. (2000) Nucleic Acids Res. 28:214). The Chothia definition is based on the location of the structural loop regions (Chothia et al. (1986) J. Mol. Biol. 196:901; Chothia et al. (1989) Nature 342:877). The AbM definition, a compromise between the Kabat and Chothia definitions, is an integral suite of programs for antibody structure modeling produced by the Oxford Molecular Group (Martin et al. (1989) Proc. Nat'l. Acad. Sci. (USA) 86:9268; Rees et al., ABMTM, a computer program for modeling variable regions of antibodies, Oxford, UK; Oxford Molecular, Ltd.). An additional definition, known as the contact definition, has been recently introduced (see MacCallum et al. (1996) J. Mol. Biol. 5:732), which is based on an analysis of available complex crystal structures.

By convention, the CDR domains in the heavy chain are referred to as H1, H2, and H3, which are numbered sequentially in order moving from the amino terminus to the carboxy terminus. The CDR-H1 is about ten to 12 residues in length and starts four residues after a Cys according to the Chothia and AbM definitions, or five residues later according to the Kabat definition. The H1 can be followed by a Trp, Trp-Val, Trp-Ile, or Trp-Ala. The length of H1 is approximately ten to 12 residues according to the AbM definition, while the Chothia definition excludes the last four residues. The CDR-H2 starts 15 residues after the end of H1 according to the Kabat and AbM definitions, which is generally preceded by sequence Leu-Glu-Trp-Ile-Gly (but a number of variations are known) and is generally followed by sequence Lys/Arg-Leu/Ile/Val/Phe/Thr/Ala-Thr/Ser/Ile/Ala. According to the Kabat definition, the length of H2 is about 16 to 19 residues, while the AbM definition predicts the length to be nine to 12 residues. The CDR-H3 usually starts 33 residues after the end of H2, is generally preceded by the amino acid sequence Cys-Ala-Arg and followed by the amino acid Gly, and has a length that ranges from three to about 25 residues.

By convention, the CDR regions in the light chain are referred to as L1, L2, and L3, which are numbered sequentially in order moving from the amino terminus to the carboxy terminus. The CDR-L1 generally starts at about residue 24 and generally follows a Cys. The residue after the CDR-L1 is always Tip, which begins one of the following sequences: Trp-Tyr-Gln, Trp-Leu-Gln, Trp-Phe-Gln, or Trp-Tyr-Leu. The length of CDR-L1 is approximately ten to 17 residues. The CDR-L2 starts about 16 residues after the end of L1 and will generally follow residues Ile-Tyr, Val-Tyr, Ile-Lys, or Ile-Phe. The CDR-L2 is about seven residues in length. The CDR-L3 usually starts 33 residues after the end of L2 and generally follows a Cys, which is generally followed by the sequence Phe-Gly-XXX-Gly and has a length of about seven to 11 residues. A binding domain of this disclosure can comprise a single CDR from a variable region of an anti-TGFβ or anti-TGFβR2, or it can comprise multiple CDRs that can be the same or different.

Thus, a binding domain of this disclosure can comprise a single CDR from a variable region of an anti-TGFβ or anti-TGFβR2, or it can comprise multiple CDRs that can be the same or different. In certain embodiments, binding domains of this disclosure comprise V_{H} and V_{L} domains specific for a TGFβ or TGFβR2 comprising framework regions and CDR1, CDR2 and CDR3 regions, wherein (a) the V_{H} domain comprises an amino acid sequence of a heavy chain CDR3; or (b) the V_{L} domain comprises an amino acid sequence of a light chain CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for a TGFβ or TGFβR2 comprising framework regions and CDR1, CDR2 and CDR3 regions, wherein (a) the V_{H} domain comprises an amino acid sequence of a heavy chain CDR1, CDR2, and CDR3; or (b) the V_{L} domain comprises an amino acid sequence of a light chain CDR1, CDR2, and CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence.

In any of the embodiments described herein comprising specific CDRs, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain, wherein each CDR has at most three amino acid changes (i.e., many of the changes will be in the framework regions); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR has at most three amino acid changes (i.e., many of the changes will be in the framework regions); or (iii) both a V_{H} domain of (i) and a V_{L} domain of (ii); or both a V_{H} domain of (i) and a V_{L} domain of (ii) wherein the V_{H} and V_{L} are from the same reference sequence.

A TGFβ antagonist domain of fusion proteins of this disclosure may be an immunoglobulin-like domain such as an immunoglobulin scaffold. Immunoglobulin scaffolds contemplated by this disclosure include a scFv, a domain antibody or a heavy chain-only antibody. In a scFv, this disclosure contemplates the heavy and light chain variable regions are joined by any linker peptide known in the art to be compatible with domain or region joinder in a binding molecule. Exemplary linkers are linkers based on the Gly₄Ser linker motif, such as (Gly₄Ser)ₙ, wherein n=1-5. If a binding domain of a fusion protein of this disclosure is based on a non-human immunoglobulin or includes non-human CDRs, the binding domain may be “humanized” according to methods known in the art.

Alternatively, a TGFβ antagonist domain of fusion proteins of this disclosure may be a scaffold other than an immunoglobulin scaffold. Other scaffolds contemplated by this disclosure present the TGFβ-specific CDR(s) in a functional conformation. Other scaffolds contemplated include, but are not limited to, an A domain molecule, a fibronectin III domain, an anticalin, an ankyrin-repeat engineered binding molecule, an adnectin, a Kunitz domain or a protein AZ domain affibody.

### IL6 Antagonists

As noted above, in certain embodiments the present disclosure provides polypeptides containing a binding region or domain that is an IL6 antagonist (e.g., preferentially inhibits IL6 trans-signaling or inhibits both IL6 cis- and trans-signaling). In certain embodiments, the present disclosure provides multi-specific fusion proteins containing a binding region or domain specific for an IL6/IL6R complex that has one or more of the following properties: (1) greater or equal affinity for an IL6/IL6R complex than for IL6 or IL6Rα alone or has greater affinity for IL6Rα alone or an IL6/IL6R complex than for IL6 alone, (2) competes with membrane gp130 for binding with a sIL6/IL6R complex or augments soluble gp130 binding to sIL6/IL6R complex, (3) preferentially inhibits IL6 trans-signaling over IL6 cis-signaling, or (4) does not inhibit signaling of gp130 family cytokines other than IL6. In certain preferred embodiments, a binding domain specific for an IL6/IL6R complex according to this disclosure has the following properties: (1) greater affinity for IL6Rα alone or an IL6xR complex than for IL6 alone, (2) augments soluble gp130 binding to sIL6/IL6R complex, (3) preferentially inhibits IL6 trans-signaling over IL6 cis-signaling, and (4) does not inhibit signaling of gp130 family cytokines other than IL6. For example, a binding region or domain specific for an IL6/IL6R complex may be an immunoglobulin variable binding domain or derivative thereof, such as an antibody, Fab, scFv, or the like. In the context of this disclosure, it should be understood that a binding region or domain specific for an IL6/IL6R complex is not gp130 as described herein.

As used herein, “IL6xR complex” or “IL6xR” refers to a complex of an IL6 with an IL6 receptor, wherein the IL6 receptor (also known as, for example, IL6Ra, IL6RA, IL6R1, and CD126) is either a membrane protein (referred to herein as mIL6R or mIL6Rα) or a soluble form (referred to herein as sIL6R or sIL6Rα). The term “IL6R” encompasses both mIL6Rα and sIL6Ra. In one embodiment, IL6xR comprises a complex of IL6 and mIL6Rα. In certain embodiments, the IL6xR complex is held together via one or more covalent bonds. For example, the carboxy terminus of an IL6R can be fused to the amino-terminus of an IL6 via a peptide linker, which is known in the art as a Hyper-IL6 (see, e.g., Fischer et al. (1997) Nat. Biotechnol. 15:142). A Hyper-IL6 linker can be comprised of a cross-linking compound, a one to 50 amino acid sequence, or a combination thereof. A Hyper-IL6 may further include a dimerization domain, such as an immunoglobulin Fc domain or an immunoglobulin constant domain sub-region. In certain embodiments, the IL6xR complex is held together via non-covalent interactions, such as by hydrogen bonding, electrostatic interactions, Van der Waal's forces, salt bridges, hydrophobic interactions, or the like, or any combination thereof. For example, an IL6 and IL6R can naturally associate non-covalently (e.g., as found in nature, or as synthetic or recombinant proteins) or each can be fused to a domain that promotes multimerization, such as an immunoglobulin Fc domain, to further enhance complex stability.

As used herein, “gp130” refers to a signal transduction protein that binds to an IL6xR complex. The gp130 protein can be in a membrane (mgp130), soluble (sgp130), or any other functional form thereof. Exemplary gp130 proteins have a sequence as set forth in GenBank Accession No. NP_{—}002175.2 or any soluble or derivative form thereof (see, e.g., Narazaki et al. (1993) Blood 82:1120 or Diamant et al. (1997) FEBS Lett. 412:379). By way of illustration and not wishing to be bound by theory, an mgp130 protein can bind to either an IL6/mILR or an IL6/sILR complex, whereas a sgp130 primarily binds with an IL6/sILR complex (see Scheller et al. (2006) Scand. J. Immunol. 63:321). Thus, certain embodiments of binding domains, or fusion proteins thereof, of the instant disclosure can inhibit IL6xR complex trans-signaling by binding with higher affinity to IL6xR than to either IL6 or IL6Rα alone and preferably by competing with sIL6xR complex binding to mgp130. A binding domain of the instant disclosure “competes” with gp130 binding to a sIL6xR when (1) a binding domain or fusion protein thereof prevents gp130 from binding a sIL6xR and the binding domain binds sIL6xR with equal or higher affinity as compared to the binding of gp130 with sIL6xR, or (2) a binding domain or fusion protein thereof enhances or promotes sgp130 binding to sIL6xR.

In one aspect, an IL6 antagonist of this disclosure has an affinity for IL6 or IL6xR complex that is at least 2-fold to 1000-fold greater than for IL6Rα alone or has an affinity for IL6Rα or IL6xR complex that is at least 2-fold to 1000-fold greater than for IL6 alone. By binding to IL6, IL6R, or IL6xR complex, an IL6 antagonist of this disclosure preferentially inhibits IL6 cis- and trans-signaling. In certain embodiments, the affinity of a binding domain for IL6 or sIL6xR complex is about the same as the affinity of gp130 for IL6xR complex—with “about the same” meaning equal or up to about 2-fold higher affinity. In certain embodiments, the affinity of the binding domain for IL6, IL6R, or IL6xR complex is higher than the affinity of gp130 for IL6xR complex by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 50-fold, at least 100-fold, 1000-fold, or greater. For example, if the affinity of gp130 for a IL6xR complex is about 2 nM (see, e.g., Gaillard et al. (1999) Eur. Cytokine Netw. 10:337), then a binding domain having at least a 10-fold higher affinity for the IL6xR complex would have a dissociation constant (K_{d}) of about 0.2 nM or less.

In further embodiments, an IL6 antagonist binding domain of this disclosure comprises a polypeptide sequence that (a) binds to a sIL6xR complex with an affinity at least 2-fold, 10-fold, 25-fold, 50-fold, 75-fold to 100-fold, 100-fold to 1000-fold higher than for either IL6 or IL6Rα alone and (b) competes with membrane gp130 for binding to sIL6xR complex or augments soluble gp130 binding to sIL6xR complex. In further embodiments, a polypeptide binding domain of this disclosure that binds to a sIL6xR complex with an affinity at least 2-fold, 10-fold, 25-fold, 50-fold, 75-fold to 100-fold, 100-fold to 1000-fold higher than for either IL6 or IL6Rα alone may also (i) more significantly or preferentially inhibit IL6 trans-signaling over IL6 cis-signaling, (ii) not inhibit signaling of gp130 cytokine family members other than IL6, (iii) preferentially inhibit IL6 trans-signaling over IL6 cis-signaling and not detectably inhibit signaling of gp130 family cytokines other than IL6, (iv) may have two or more of these properties, or (v) may have all of these properties.

In certain embodiments, a polypeptide IL6 antagonist binding domain of this disclosure binds to a sIL6xR complex with an affinity at least 2-fold to 1000-fold higher than for either IL6 or IL6Rα alone and more significantly or preferentially inhibits IL6 trans-signaling over IL6 cis-signaling. To “preferentially inhibit IL6 trans-signaling over IL6 cis-signaling” refers to altering trans-signaling to an extent that sIL6xR activity is measurably decreased while the decrease in IL6 cis-signaling is not substantially altered (i.e., meaning inhibition is minimal, non-existent, or not measurable). For example, a biomarker for sIL6xR activity (e.g., acute phase expression of antichymotrypsin (ACT) in HepG2 cells) can be measured to detect trans-signaling inhibition. A representative assay is described by Jostock et al. (Eur. J. Biochem., 2001)—briefly, HepG2 cells can be stimulated to overexpress ACT in the presence of sIL6xR (trans-signaling) or IL6 (cis-signaling), but adding spg130 will inhibit the overexpression of ACT induced by sIL6xR while not substantially affecting IL6 induced expression. Similarly, a polypeptide binding domain of this disclosure that preferentially inhibits IL6 trans-signaling over IL6 cis-signaling will inhibit the overexpression of ACT induced by sIL6xR (i.e., inhibit trans-signaling) while not substantially affecting IL6 induced expression (i.e., not measurably decrease cis-signaling). This and other assays known in the art can be used to measure preferential inhibition of IL6 trans-signaling over IL6 cis-signaling (see, e.g., other biomarkers described in Sporri et al. (1999) Int. Immunol. 11:1053; Mihara et al. (1995) Br. J. Rheum. 34:321; Chen et al. (2004) Immun. 20:59).

In further embodiments, signaling by gp130 family cytokines other than IL6 is not substantially inhibited by binding domain polypeptides or multi-specific fusion proteins thereof of this disclosure. For example, cis- and trans-signaling by an IL6xR complex via gp130 will be inhibited, but signaling by one or more other gp130 family cytokines will be minimally affected or unaffected, such as signaling via leukemia inhibitory factor (LIF), ciliary neurotropic factor (CNTF), neuropoietin (NPN), cardiotropin like cytokine (CLC), oncostatin M (OSM), IL-11, IL-27, IL-31, cardiotrophin-1 (CT-1), or any combination thereof.

It will be appreciated by those skilled in the art that the preferred in vivo half-life of a binding domain of this disclosure is on the order of days or weeks, but while the binding domain concentration may be low, the target may be plentiful as both IL6 and sIL6 production can be quite elevated in disease states (see, e.g., Lu et al. (1993) Cytokine 5:578).

Thus, in certain embodiments, a binding domain of this disclosure has a k_{OFF} of about 10⁻⁵/sec (e.g., about a day) or less. In certain embodiments, the k_{OFF} can range from about 10⁻¹/sec, about 10⁻²/sec, about 10⁻³/sec, about 10⁻⁴/sec, about 10⁻⁵/sec, about 10⁻⁶/sec, about 10⁻⁷/sec, about 10⁻⁸/sec, about 10⁻⁹/sec, about 10⁻¹⁰/sec, or less.

In an illustrative example, binding domains of this disclosure specific for an IL6 or IL6xR complex were identified in a Fab phage library of fragments (see Hoet et al. (2005) Nature Biotechnol. 23:344) by screening for binding to a synthetic IL6xR complex. The synthetic IL6xR complex used for this screening comprises a structure of N-IL6Rα(frag)-L1-IL6(frag)-L2-ID-C, wherein N is the amino-terminus and C is the carboxy-terminus, IL6Rα(frag) is a fragment of full length IL6Rα, IL6(frag) is a fragment of IL6, L1 and L2 are linkers, and ID is an intervening or dimerization domain, such as an immunoglobulin Fc domain.

More specifically, an IL6xR (which is a form of Hyper IL6) used to identify the binding domains specific for IL6xR complex has a structure, from amino-terminus to carboxy-terminus, as follows: (a) a central fragment of 212 amino acids from IL6Rα that is missing the first 110 amino acids of the full length protein and a carboxy-terminal portion that will depend on the isoform used (see GenBank Accession No. NP_{—}000556.1, isoform 1 or NP_{—}852004.1, isoform 2) fused to (2) a linker of G₃S that is in turn fused to (3) a 175 amino acid carboxy-terminal fragment of IL6 (i.e., missing the first 27 amino acids of the full length protein; GenBank Accession No. NP_{—}000591.1) that is in turn fused to (4) a linker that is an IgG2A hinge as set forth in SEQ ID NO:589, which is finally fused to a dimerization domain comprised of an immunoglobulin G1 (IgG1) Fc domain. In certain embodiments, the dimerization domain comprised of an IgG1 Fc domain has one or more of the following amino acids mutated (i.e., have a different amino acid at that position): leucine at position 234 (L234), leucine at position 235 (L235), glycine at position 237 (G237), glutamate at position 318 (E318), lysine at position 320 (K320), lysine at position 322 (K322), or any combination thereof (EU numbering). For example, any one of these amino acids can be changed to alanine. In a further embodiment, an IgG1 Fc domain has each of L234, L235, G237, E318, K320, and K322 (according to Kabat numbering) mutated to an alanine (i.e., L234A, L235A, G237A, E318A, K320A, and K322A, respectively).

In one embodiment, an IL6xR complex used to identify the IL6 antagonist binding domains of this disclosure has an amino acid sequence as set forth in SEQ ID NO:606. In certain embodiments, there are provided polypeptides containing a binding domain specific for an IL6xR complex, wherein the IL6xR is a sIL6xR and has the amino acid sequence as set forth in SEQ ID NO:606. In further embodiments, polypeptides containing a binding domain specific for an IL6xR complex (1) have greater or equal affinity for an IL6xR complex than for IL6 or IL6Rα alone, or have greater affinity for IL6Rα alone or an IL6xR complex than for IL6 alone, (2) compete with membrane gp130 for binding with a sIL6xR complex or augment soluble gp130 binding to sIL6xR complex, (3) preferentially inhibit IL6 trans-signaling over IL6 cis-signaling, or (4) do not inhibit signaling of gp130 family cytokines other than IL6, (5) have any combination thereof of properties (1)-(4), or (6) have all of the properties of (1)-(4). Other exemplary IL6xR complexes that may be used to identify binding domains of the instant disclosure or used as a reference complex to measure any of the aforementioned binding properties are described, for example, in US Patent Publication Nos. 2007/0172458; 2007/0031376; and U.S. Pat. Nos. 7,198,781; 5,919,763.

In some embodiments, IL6 antagonist binding domains of this disclosure comprise V_{H} and V_{L} domains specific for an IL6, IL6R, or IL6xR complex as described herein, and preferably human IL6, human IL6R, or human IL6xR complex. In certain embodiments, the V_{H} and V_{L} domains are rodent, (e.g., mouse, rat), humanized, or human. Examples of binding domains containing such V_{H} and V_{L} domains specific for IL6, IL6R, or IL6xR are set forth in SEQ ID NOS:435-496 and 373-434, respectively. In further embodiments, there are provided polypeptide binding domains specific for an IL6xR wherein the binding domain comprises a sequence that is at least 90%, at least 91%, at least 92%, at least 93%, at least 94%, at least 95%, at least 96%, at least 97%, at least 98%, at least 99%, at least 99.5%, or at least 100% identical to the amino acid sequence of one or more light chain variable regions (V_{L}) or to one or more heavy chain variable regions (V_{H}), or both, as set forth in SEQ ID NOS:373-434 and 435-496, respectively, wherein each CDR has up to three amino acid changes (i.e., many of the changes are found in one or more of the framework regions).

In further embodiments, binding domains of this disclosure comprise V_{H} and V_{L} domains specific for an IL6xR as set forth in SEQ ID NOS:435-496 and 373-434, respectively, which are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of such V_{H} domain, V_{L} domain, or both, wherein each CDR has zero, one, two, or three amino acid changes. For example, the amino acid sequence of a V_{H} domain, V_{L} domain, or both of this disclosure can be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of V_{H} domain (e.g., amino acids 512 to 636), V_{L} domain (e.g., amino acids 652 to 759), or both, respectively, from an exemplary xceptor molecule containing binding domain TRUE-1002 (see SEQ ID NO:608), wherein each CDR has zero, one, two, or three amino acid changes.

In any of these or other embodiments described herein, the V_{L} and V_{H} domains may be arranged in either orientation and may be separated by up to about a ten amino acid linker as disclosed herein or any other amino acid sequence capable of providing a spacer function compatible with interaction of the two sub-binding domains. In certain embodiments, a linker joining the V_{H} and V_{L} domains comprises an amino acid sequence as set forth in SEQ ID NO:497-604 and SEQ ID NO:1223-1228, such as Linker 47 (SEQ ID NO:543) or Linker 80 (SEQ ID NO:576).

In further embodiments, IL6 antagonist binding domains of this disclosure may comprise one or more complementarity determining region (“CDR”), or multiple copies of one or more such CDRs, which have been obtained, derived, or designed from variable regions of an anti-IL6, anti-IL6R, or anti-IL6xR complex scFv or Fab fragment or from heavy or light chain variable regions thereof. Thus, a binding domain of this disclosure can comprise a single CDR from a variable region of an IL6 or anti-IL6xR, or it can comprise multiple CDRs that can be the same or different. In certain embodiments, IL6 antagonist binding domains of this disclosure comprise V_{H} and V_{L} domains comprising framework regions and CDR1, CDR2 and CDR3 regions, wherein (a) the V_{H} domain comprises the amino acid sequence of a heavy chain CDR3 found in any one of SEQ ID NOS:435-496; or (b) the V_{L} domain comprises the amino acid sequence of a light chain CDR3 found in any one of SEQ ID NOS:373-434; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for an IL6xR complex comprising framework regions and CDR1, CDR2 and CDR3 regions, wherein (a) the V_{H} domain comprises the amino acid sequence of a heavy chain CDR1, CDR2, and CDR3 found in any one of SEQ ID NOS:435-496; or (b) the V_{L} domain comprises the amino acid sequence of a light chain CDR1, CDR2, and CDR3 found in any one of SEQ ID NOS:373-434; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence. Exemplary light and heavy chain variable domain CDRs directed against IL6, IL6R, or IL6xR complex are provided in SEQ ID NO:1-186 and 1187-1192, and 187-372 and 1193-1198, respectively.

Amino acid sequences of IL6 antagonist light chain variable regions are provided in SEQ ID NO:373-434 and 1199-1204, with the corresponding heavy chain variable regions being provided in SEQ ID NO:435-496 and 1205-1210, respectively.

In any of the embodiments described herein comprising specific CDRs against IL6, IL6R, or IL6xR, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain found in any one of SEQ ID NOS:435-496; or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain found in any one of SEQ ID NOS:373-434; or (iii) both a V_{H} domain of (i) and a V_{L} domain of (ii); or both a V_{H} domain of (i) and a V_{L} domain of (ii) wherein the V_{H} and V_{L} are from the same reference sequence.

In certain embodiments, a binding domain of this disclosure may be an immunoglobulin-like domain, such as an immunoglobulin scaffold. Immunoglobulin scaffolds contemplated in this disclosure include a scFv, Fab, a domain antibody, or a heavy chain-only antibody. In further embodiments, there are provided anti-IL6 or anti-IL6xR antibodies (e.g., non-human such as mouse or rat, chimeric, humanized, human) or Fab fragments or scFv fragments that have an amino acid sequence that is at least 80%, 85%, 90%, 91%, 92%, 93%, 94%, 95%, 96%, 97%, 98%, 99%, or 100% identical to the amino acid sequence of a V_{H} and V_{L} domain set in any one of SEQ ID NOS:435-496 and 373-434, respectively, which may also have one or more of the following properties: (1) have greater or equal affinity for an IL6xR complex than for IL6 or IL6Rα alone, or have greater affinity for IL6Rα alone or an IL6xR complex than for IL6 alone, (2) compete with membrane gp130 for binding with a sIL6xR complex or augment soluble gp130 binding to sIL6xR complex, (3) preferentially inhibit IL6 trans-signaling over IL6 cis-signaling, or (4) do not inhibit signaling of gp130 family cytokines other than IL6. Such antibodies, Fabs, or scFvs can be used in any of the methods described herein. In certain embodiments, the present disclosure provides polypeptides containing a binding domain that is an IL6 antagonist (i.e., can inhibit IL6 cis- and trans-signaling). In further embodiments, an IL6 antagonist according to this disclosure does not inhibit signaling of gp130 family cytokines other than IL6. Exemplary IL6 antagonists include binding domains specific for an IL6 or IL6xR, such as an immunoglobulin variable binding domain or derivative thereof (e.g., an antibody, Fab, scFv, or the like).

Alternatively, binding domains of this disclosure may be part of a scaffold other than an immunoglobulin. Other scaffolds contemplated include an A domain molecule, a fibronectin III domain, an anticalin, an ankyrin-repeat engineered binding molecule, an adnectin, a Kunitz domain, or a protein AZ domain affibody.

### IL10 Antagonists

In certain embodiments the present disclosure provides polypeptides containing a binding region or domain that is an IL10 antagonist (i.e., can inhibit IL10 signaling). Exemplary IL10 antagonists include binding domains specific for an IL10 or IL10R1, such as an immunoglobulin variable binding domain or derivative thereof (e.g., an antibody, Fab, scFv, or the like), or an IL10R1 ectodomain.

IL10 is a member of a cytokine superfamily that share an alpha-helical structure. Although no empirical evidence exists, it has been suggested that all possess six alpha-helices (Fickenscher, H. et al., 2002, Trends Immunol. 23: 89). IL10 has four cysteines, only one of which is conserved among family members. Since IL10 demonstrates a V-shaped fold that contributes to its dimerization, it appears that disulfide bonds are not critical to this structure. Amino acid identity of family members to IL10 ranges from 20% (IL-19) to 28% (IL-20) (Dumouter et al., 2002, Eur. Cytokine Netw. 13: 5).

IL10 was first described as a Th2 cytokine in mice that inhibited IFN-α and GM-CSF cytokine production by Th1 cells (Moore et al., 2001, Annu Rev. Immunol. 19: 683; Fiorentino et al., 1989, J. Exp. Med. 170:2081).

Human IL10 is 178 amino acids in length with an 18 amino acid signal sequence and a 160 amino acid mature segment and a molecular weight of approximately 18 kDa (monomer). Human IL10 contains no potential N-linked glycosylation site and is not glycosylated (Dumouter et al., 2002, Eur. Cytokine Netw. 13: 5; Vieira et al., 1991, Proc. Natl. Acad. Sci. USA 88:1172). It contains four cysteine residues that form two intrachain disulfide bonds. The length of α-helices A to F in human IL10 are 21, 8, 19, 20, 12 and 23 amino acids, respectively. Helices A to D of one monomer noncovalently interact with helices E and F of a second monomer, forming a noncovalent V-shaped homodimer. Functional areas have been mapped on the IL10 molecule. In the N-terminus, pre-helix A residues no. 1-9 are involved in mast cell proliferation, while in the C-terminus, helix F residues no. 152-160 mediate leukocyte secretion and chemotaxis.

Cells known to express IL10 include CD8+ T cells, microglia, CD14+ (but not CD16+) monocytes, Th2 CD4+ cells (mice), keratinocytes, hepatic stellate cells, Th1 and Th2 CD4+ T cells (human), melanoma cells, activated macrophages, NK cells, dendritic cells, B cells (CD5+ and CD19+) and eosinophils.

On T cells, the initial observations of IL10 inhibition of IFN-gamma production are now believed to be an indirect effect mediated by accessory cells. Additional effects on T cells, however, include: IL10 induced CD8+ T cell chemotaxis, an inhibition of CD4+ T cell chemotaxis towards IL-8, suppression of IL-2 production following activation, an inhibition of T cell apoptosis via Bcl-2 up-regulation, and an interruption of T cell proliferation following low antigen exposure accompanied by B7/CD28 costimulation (Akdis et al., 2001, Immunology 103: 131).

On B cells, IL10 has a number of related, yet distinct functions. In conjunction with TNF-β and CD40L, IL10 induces IgA production in naïve (IgD+) B cells. It is believed that TGF-β/CD40L promotes class switching while IL10 initiates differentiation and growth. When TGF-β is not present, IL10 cooperates with CD40L in inducing IgG1 and IgG3 (human), and thus may be a direct switch factor for IgG subtypes. IL10 has divergent effects on IL-4 induced IgE secretion. If IL10 is present at the time of IL-4 induced class switching, it reverses the effect; if it is present after IgE commitment, it augments IgE secretion. CD27/CD70 interaction in the presence of IL10 promotes plasma cell formation from memory B cells (Agematsu et al., 1998, Blood 91: 173).

Mast cells and NK cells are also impacted by IL10. On mast cells, IL10 induces histamine release while blocking GM-CSF and TNF-α release. This effect may be autocrine as IL10 is known to be released by mast cells in rat. As evidence of its pleiotrophic nature, IL10 has the opposite effects on NK cells. Rather than blocking TNF-α and GM-CSF production, IL10 actually promotes this function on NK cells. In addition, it potentiates IL-2 induced NK cell proliferation and facilitates IFN-γ secretion in NK cells primed by IL-18. In concert with both IL-12 and/or IL-18, IL10 potentiates NK cell cytotoxicity (Cai et al., 1999, Eur. J. Immunol. 29: 2658).

IL10 has a pronounced anti-inflammatory impact on neutrophils. It inhibits the secretion of the chemokines MIP-1α, MIP-1β and IL-8, and blocks production of the proinflammatory mediators IL-1β and TNF-α. In addition, it decreases the ability of neutrophils to produce superoxide, and as a result interferes with PMN-mediated antibody-dependent cellular cytotoxicity. IL10 also blocks IL-8 and fMLP-induced chemotaxis, possibly via CXCR1 (Vicioso et al., 1998 Eur. Cytokine Netw. 9: 247).

On dendritic cells (DCs), IL10 generally exhibits immunosuppressive effects. It would appear to promote CD14+ macrophage differentiation at the expense of DCs. Macrophages, while phagocytic, are poor antigen-presenting cells. IL10 seems to decrease the ability of DCs to stimulate T cells, particularly for Th1 type cells. How IL10 accomplishes this is unclear, as the data within the literature is conflicting. Relative to MHC-II expression, it can be down-regulated, unchanged, or up-regulated (Sharma et al., 1999, J. Immunol. 163:5020). With respect to B7-1/CD80, IL10 will either up-regulate or down-regulate its expression. B7-2/CD86 plays a key role in T cell activation. For this molecule, IL10 is involved in both up-regulation and down-regulation. Perhaps the most significant modulation, however, occurs with CD40 (IL10 seems to reduce its expression). At the regional level, IL10 may block immunostimulation by inhibiting Langerhans cell migration in response to proinflammatory cytokines. Alternatively, IL10 blocks an inflammation-induced DC maturation step that normally involves CCR1, CCR2 and CCR5 down-regulation and CCR7 up-regulation. This blockage, with retention of CCR1, CCR2 and CCR5, results in a failure of DCs to migrate to regional nodes. The result is an immobile DC that will not stimulate T cells but will bind (and clear) proinflammatory chemokines without responding to them (D-Amico et al., 2000 Nat. Immunol. 1:387).

On monocytes, IL10 has a number of documented effects. For example, IL10 seems to clearly reduce cell surface MHC-II expression. It also inhibits IL-12 production following stimulation. While it promotes a monocyte to macrophage transition in conjunction with M-CSF, the phenotype of the macrophage is not clear (i.e. CD16+/cytotoxic vs. CD16−). IL10 also reduces monocyte GM-CSF secretion and IL-8 production, while promoting IL-1ra release (Gesser et al., 1997, Proc. Natl. Acad. Sci. USA 94:14620). Hyaluronectin, a connective tissue component, is now known to be secreted by monocytes in response to IL10. This may have some importance in cell migration, particularly tumor cell metastases, where hyaluronectin is known to interrupt cell migration through extracellular space (Gesser et al., 1997).

Human IL10R1 is a 90-110 kDa, single-pass type I transmembrane glycoprotein that is expressed on a limited number of cell types (Liu et al., 1994, J. Immunol. 152:1821). Weak expression is seen in pancreas, skeletal muscle, brain, heart and kidney. Placenta, lung, and liver showed intermediate levels of expression, while monocytes, B-cells, large granular lymphocytes and T-cells express high levels (Liu et al., 1994). The expressed protein is a 578 amino acid protein that contains a 21 amino acid signal peptide, a 215 amino acid extracellular region, a 25 amino acid transmembrane segment, and a 317 amino acid cytoplasmic domain. There are two FNIII motifs within the extracellular region and a STAT3 docking site plus a JAK1 association region within the cytoplasmic domain (Kotenko et al., 2000 Oncogene 19:2557; Kotenko et al., 1997, EMBO J. 16:5894). IL10R1 binds human IL10 with a Kd of about 200 pM.

In some embodiments, binding domains of this disclosure comprise V_{H} and V_{L} domains specific for an IL10 or an IL10R1. In certain embodiments, the V_{H} and V_{L} domains are rodent (e.g., mouse, rat), humanized, or human. Examples of binding domains containing such V_{H} and V_{L} domains specific for IL10 include, but are not limited to, those disclosed in US Patent Application Publication no. US 2007/0178097A1. Binding domains of this disclosure may also, or alternatively, comprise an IL10R1 ectodomain as shown, for example, in SEQ ID NO:745, or a fragment thereof. In further embodiments, there are provided polypeptide binding domains specific for IL10, wherein the binding domain comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to an amino acid sequence of SEQ ID NO:745 or to amino acids 22-401 of SEQ ID NO:745, wherein the polypeptide binding domain binds to IL10 and inhibits the activity thereof.

### GITR Agonists

In certain embodiments the present disclosure provides polypeptides containing a binding region or domain that is a GITR agonist (i.e., can increase GITR signaling). Exemplary GITR agonists include binding domains specific for a GITR or GITRL, such as an immunoglobulin variable binding domain or derivative thereof (e.g., an antibody, Fab, scFv, or the like), or a GITRL ectodomain.

Glucocorticoid-induced tumor necrosis factor receptor (GITR; also known as AITR) is a type I transmembrane protein and a member of the TNF receptor superfamily (Nocentini et al., (2007) Eur. J. Immunol. 37:1165-9). The cytoplasmic domain has homology to the cytoplasmic domain of 4-1BB and CD27. GITR is expressed in peripheral blood T cells, bone marrow, thymus, spleen, and lymph nodes, and is constitutively expressed in CD4⁺CD25⁺ regulatory T cells (Kwon et al., (2003) Exp. Mol. Med. 35:13). In addition, it is constitutively expressed at low levels in natural killer (NK) cells and is induced upon stimulation by either Toll-like receptor ligand or IL-15 (Liu et al., (2008) J. Biol. Chem. 283:8202).

Expression of GITR is increased following T cell activation. Activation of GITR coactivates effector T lymphocytes and modulates regulatory T cell activity. Binding of GITR to its ligand GITRL has been shown to render CD4⁺CD25⁻ effector T cells resistant to the inhibitory effects of CD4⁺CD25⁺ regulatory T cells.

GITR ligand (GITRL) is a type II membrane protein. It is 173 amino acids long with a predicted molecular weight of 20 kDa. The experimental molecular weight of 25-28 kDa is suggestive of glycosylation. GITRL is expressed in antigen presenting cells (APC) and is constitutively expressed in human umbilical vein endothelial cells (Nocentini et al. Ibid). It is not, however, expressed in resting or stimulated T cells, B cell lines, or peripheral blood mononuclear cells.

The GITR/GITRL system has been shown to increase resistance to tumors and viral infections (Nocentini et al., ibid). Specifically, the anti-GITR monoclonal antibody DTA-1 was shown to inhibit regulatory T cell-dependent suppression and enhance T cell responses. Administration of DTA-1 in mice induced B16 melanoma tumor rejection. GITR is also involved in autoimmune/inflammatory processes and regulates leukocyte extravasation. GITR−/− mice exhibit decreased sensitivity to inflammatory disease conditions, indicating a positive role for GITR in inflammation.

In some embodiments, binding domains of this disclosure comprise V_{H} and V_{L} domains specific for a GITR or a GITRL. In certain embodiments, the V_{H} and V_{L} domains are rodent (e.g., mouse, rat), humanized, or human. Examples of binding domains containing such V_{H} and V_{L} domains specific for GITR include, but are not limited to, those disclosed in US Patent Application Publication no. US 2007/0098719A1. Binding domains of this disclosure may also, or alternatively, comprise a GITRL ectodomain (e.g. amino acids 74-181 of Genbank Accession NP_{—}005083.2 (SEQ ID NO:746) or a fragment thereof. In further embodiments, there are provided polypeptide binding domains specific for GITR, wherein the binding domain comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to amino acids 74-181 of SEQ ID NO:746, wherein the polypeptide binding domain binds to GITR and increases the activity thereof.

### VEGF Antagonists

In certain embodiments the present disclosure provides polypeptides containing a binding region or domain that is a VEGF antagonist (i.e., can inhibit VEGF signaling). Exemplary VEGF antagonists include binding domains specific for a VEGF or VEGFR2, such as an immunoglobulin variable binding domain or derivative thereof (e.g., an antibody, Fab, scFv, or the like), or a VEGFR2 ectodomain.

Vascular endothelial growth factor (VEGF or VEGF-A) is an evolutionarily conserved homodimeric glycoprotein and a potent endothelial cell—specific mitogen that plays a critical role in angiogenesis and vasculogenesis (Lee et al. (2007) PLOS Medicine 6:1101-1116). VEGF induces various intracellular signaling and physiologic responses that are essential for angiogenesis, such as intracellular Ca²⁺ influx, chemotaxis (migration), expression of plasminogen activators, urokinase receptor and collagenases, and vascular permeability. Its biological effects are elicited through two high-affinity receptor tyrosine kinases, namely VEGF receptors 1 (VEGFR1) and 2 (VEGFR2), which are mainly expressed in endothelial cells.

VEGFA is a secreted protein that is a homodimer linked by disulfide bonds. It is also found as heterodimer with P1GF. Alternative splicing of VEGF mRNA results in various isoforms, which include VEGF121, VEGF145, VEGF165, VEGF189 and VEGF206, in humans and VEGF120, VEGF164 and VEGF188 in mice. Studies of genetically engineered mice expressing only one VEGF isoform indicate that VEGF isoforms have distinct yet some overlapping roles in vascular development and function as evidenced by tissue-specific vascular defects in these mice. The VEGF isoforms display differences in their biochemical properties, including receptor binding with VEGF165 and VEGF188 but not VEGF120 binding to neuropilins and heparan sulfate. The differential affinity to heparan sulfate is important in their binding to VEGFR1 and VEGF2, as heparan sulfate can mediate the binding and transactivation of these receptors. Furthermore, differential binding to heparan sulfate is reported to lead to different VEGF actions, including endothelial cell survival, adhesion and vascular branch formation. Both VEGF164 and VEGF188 bind heparan sulfate, making them partially or fully cell-bound, respectively, whereas VEGF120 does not bind heparan sulfate, and is freely diffusible.

The VEGF isoforms display tissue-specific patterns of expression. The VEGF189, VEGF-165 and VEGF-121 isoforms are widely expressed, whereas the VEGF206 and VEGF-145 are uncommon. Its expression is regulated by growth factors, cytokines, gonadotropins, nitric oxide, hypoxia, hypoglycemia and oncogenic mutations.

The classical role of VEGF in tumor progression is as a positive regulator of angiogenesis, the process of forming new capillaries from preexisting blood vessels. Tumor growth is highly dependent on the ability of tumors to induce their own vascularization. VEGF expression has been reported in a number of cancer cell lines and in several clinical specimens derived from breast, brain, and ovarian cancers. Thus, antagonism of VEGF can effectively prevent tumor growth through incomplete blood vessel formation. VEGF exerts its effects on endothelial cells in a paracrine mode after its release by other cells such as tumor cells, or in an autocrine manner in VEGF-producing endothelial cells. VEGF binds to its cognate receptors VEGFR1 (also known as FLT1), VEGFR2 (also known as KDR or FLK1), and neuropilin 1 (NRP1).

VEGF expression in the adult is cell-type specific and is controlled at many levels from transcription to translation, and is upregulated in tumors and in various pathologic states. One of the best-characterized stimuli of VEGF transcription is hypoxia, which acts by stabilization of the hypoxia-inducible factor-1 alpha (HIF1α) transcription factor. Hypoxic regulation of VEGF also takes place post-transcriptionally via mRNA stabilization. VEGF expression is induced by other growth factors and cytokines including IGF-1, Il-6, Il-1, PDGF, TNF-α, TGF-β and FGF-4. In addition, VEGF expression is also stimulated by physical forces, including stretch, with one putative transcription factor being the Kruppel like factor-2. Analysis of the VEGF promoter reveals many other potential transcription factor responsive elements, of which several pathways have been elucidated, for example EGF and HGF signaling via the SP1 responsive element.

Members of the VEGF family promote two very important processes in vivo, angiogenesis and lymphangiogenesis, which involve growth of new blood and lymphatic vessels from pre-existing vasculature, respectively. These processes control the normal processes of wound healing, ovarian-follicular development, endometrium growth and pathological processes such as retinopathies, rheumatoid arthritis and solid tumor growth. A newly identified splice variant of VEGF, VEGF165b, is postulated to have an inhibitory effect on angiogenesis. Lymphangiogenesis is correlated with lymph node metastasis and cancer spread via the lymphatic system.

VEGF activities are mediated by high-affinity receptor tyrosine kinases expressed primarily in endothelial cells. These are: VEGFR-1 (Flt-1) and VEGFR-2 (Flk-1/KDR), which are mainly expressed by blood vessel endothelial cells and VEGFR-3 (Flt-4) expressed in lymphatic endothelial cells. These receptors are characterized by seven extracellular immunoglobulin-like domains, which bind the growth factor, followed by a single membrane-spanning region and a conserved intracellular tyrosine kinase domain interrupted by a kinase insert sequence. These receptors are themselves enzymes and once activated by ligand binding, they dimerize and undergo autophosphorylation. This step enhances the capacity of the receptor to directly activate other target proteins by phosphorylating them on specific tyrosine residues.

The VEGF-kinase ligand/receptor signaling system plays a key role in vascular development and regulation of vascular permeability. In case of HIV-1 infection, the interaction with extracellular viral Tat protein seems to enhance angiogenesis in Kaposi's sarcoma lesions.

Although VEGF binds to VEGFR1, VEGFR2, Nip-1 and Nrp-2, its main signaling receptor in the endothelium is VEGFR2. VEGFR2 belongs to the family of receptor tyrosine kinases, and upon VEGF binding, there is dimerization and activation of the tyrosine kinase, resulting in phosphorylation of specific tyrosine residues on the cytoplasmic tail, which in turn promotes docking of signal transducing molecules. VEGFR2 is responsible for initiating signal transduction pathways within endothelial cells. Following the binding of VEGF to VEGFR2, VEGF mediates its effects on proliferation, survival, adhesion, migration, capillary morphogenesis, and gene expression in endothelial cells. VEGFR1 has a relatively minor role in VEGF-mediated signal transduction as compared to VEGFR2, since its kinase activity is 10-fold less than that of VEGFR2. Breast cancer cell lines express both VEGF and the VEGF receptors VEGFR1, VEGFR2, and NRP1. Recent studies have shown that VEGF acts as an autocrine growth and survival factor for VEGF receptor-expressing tumor cells. However, the mechanism by which VEGF mediates the survival of tumor cells needs to be investigated in depth (Lee et al., 2007, PLOS Medicine 6: 1101-1116).

Although VEGFR1 is also expressed by endothelial cells (EC), it is believed to act primarily to modulate VEGFR2 signaling. Mitogenesis, chemotaxis, cell survival and changes in the morphology of endothelial cells are mainly mediated by VEGFR-2. The mitogenic signal is induced by activation of the Raf-Mek-Erk pathway, while the antiapoptotic effects and chemotaxis are mediated by PI3K/Akt activation. VEGF binding to VEGFR-2 also results in activation of several integrins, which are adhesion molecules involved in angiogenesis, in a PI3K/Akt dependent manner. Apart from being expressed in endothelial cells, VEGFR-2 is also found in haematopoietic stem cells, where it increases their survival, and in retinal progenitor cells, where it plays a critical role in neurogenesis and vasculogenesis.

In some embodiments, binding domains of this disclosure comprise V_{H} and V_{L} domains specific for a VEGF or a VEGFR2. In certain embodiments, the V_{H} and V_{L} domains are rodent (e.g., mouse, rat), humanized, or human. Examples of binding domains containing such V_{H} and V_{L} domains specific for VEGF include, but are not limited to, those disclosed in US Patent Application Publication no. US 2007/0141065A1. Binding domains of this disclosure may also, or alternatively, comprise a VEGFR2 ectodomain (see, Genbank Accession NP_{—}002244.1, SEQ ID NO:747) or a fragment thereof. In further embodiments, there are provided polypeptide binding domains specific for VEGF, wherein the binding domain comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to an amino acid sequence of SEQ ID NO:747, wherein the polypeptide binding domain binds to VEGF and inhibits the activity thereof.

### TNFα Antagonists

In certain embodiments the present disclosure provides polypeptides containing a binding region or domain that is a TNFα antagonist (i.e., can inhibit TNFα signaling). Exemplary TNFα antagonists include binding domains specific for a TNFα, such as an immunoglobulin variable binding domain or derivative thereof (e.g., an antibody, Fab, scFv, or the like), or a TNFR1 or TNFR2 ectodomain.

Tumor Necrosis Factor Receptor (TNFR) is a member of the tumor necrosis factor receptor superfamily and is the receptor for Tumor Necrosis Factor-α (TNFα), also known as CD120 or cachectin. There are two variants of this cytokine receptor, TNFR1 and TNFR2, (CD120a receptor and CD120b receptor). TNFR1 (Genbank accession no. NP_{—}001056.1) has a molecular weight of about 55 KD and is therefore sometimes referred to as p55. A TNFR domain that may be used as a TNFα binding domain in the disclosed fusion proteins is located at amino acids 44-149 of the TNFR1 sequence. TNFR2 (Genbank accession no. NP_{—}001057.1) has a molecular weight of about 75 KD and is therefore sometimes referred to as p75. A TNFR domain that may be used as a TNFα binding domain in the disclosed fusion proteins is located at amino acids 40-141 of the TNFR2 sequence.

A majority of cell types and tissues appear to express both TNF receptors. Both exist in cell surface as well as soluble forms and both are active in signal transduction, although they are able to mediate distinct cellular responses. TNFR1 appears to be responsible for signaling most TNF responses. Among other activities, TNFR2 stimulates thymocyte proliferation, activates NF-κβ, and is an accessory to TNFR1 in the signaling of responses primarily mediated by TNF-R1, like cytotoxicity.

TNF antagonists, such as anti-TNF antibodies, can positively affect various inflammatory conditions. For example, infliximab is indicated in the United States for the treatment of rheumatoid arthritis, Crohn's disease, ankylosing spondylitis, psoriatic arthritis, plaque psoriasis, and ulcerative colitis. Recently, perispinal delivery of the TNFα inhibitor etanercept has been shown to reduce symptoms in patients with Alzheimer's disease (Tobinick and Gross (2008) BMC Neurol. 8:27-36; Griffin (2008) J. Neuroinflammation, 5:3-6).

According to REMICADE® (infliximab) prescribing information, biological activities attributed to TNF include: induction of pro-inflammatory cytokines such as interleukins (IL) 1 and 6, enhancement of leukocyte migration by increasing endothelial layer permeability and expression of adhesion molecules by endothelial cells and leukocytes, activation of neutrophil and eosinophil functional activity, induction of acute phase reactants and other liver proteins, as well as tissue degrading enzymes produced by synoviocytes and/or chondrocytes.

In some embodiments, binding domains of this disclosure comprise V_{H} and V_{L} domains specific for a TNFα. In certain embodiments, the V_{H} and V_{L} domains are human. Examples of binding domains containing such V_{H} and V_{L} domains specific for TNFα include, but are not limited to, those disclosed in US Patent Application Publication no. US 2007/0249813. Binding domains of this disclosure may also, or alternatively, comprise a TNFR1 ectodomain (see, Genbank Accession NP_{—}001056.1, SEQ ID NO:749) or a fragment thereof, or a TNFR2 ectodomain (see, Genbank Accession NP_{—}001057.1, SEQ ID NO:748) or a fragment thereof. TNFR1 and TNFR2 ectodomains are described in US Patent Application Publication no. US 2007/0128177. In further embodiments, there are provided polypeptide binding domains specific for TNFα, wherein the binding domain comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to an amino acid sequence of SEQ ID NO:748 or 749, wherein the polypeptide binding domain binds to TNFα and inhibits the activity thereof.

### HGF Antagonists

As noted above, in certain embodiments the present disclosure provides polypeptides containing a binding region or domain that is a HGF antagonist (i.e., can inhibit HGF signaling). Exemplary HGF antagonists include binding domains specific for a HGF, such as an immunoglobulin variable binding domain or derivative thereof (e.g., an antibody, Fab, scFv, or the like), or a c-Met ectodomain or sub-domain thereof (e.g., a Sema domain, a PSI domain, or both domains of c-Met).

The tyrosine kinase receptor c-Met (also known as the hepatocyte growth factor receptor, HGFR, because hepatocyte growth factor (HGF) is one of its ligands) is active during the normal processes of embryogenesis and tissue repair. In both of these processes, cells dissociate from neighboring cells and enter the bloodstream. In the bloodstream, c-Met-induced protection from apoptosis and ability to grow in an anchorage-independent manner allow the cells to survive until they extravasate, proliferate and eventually differentiate. In tissue repair, c-Met is involved in the process of epithelial-mesenchymal transition when epithelial cells adjacent to the injury detach, change shape and migrate toward the injured area where they proliferate and reconstitute the epithelial layer.

However, when c-Met is constitutively activated, the cells expressing it become tumorigenic and metastatic. Constitutive c-Met activation has been demonstrated to occur by multiple mechanisms. The most common is over-expression of the receptor, which occurs as a result of c-Met gene amplification (e.g., in colectoral tumors), enhanced c-Met transcription induced by other oncogenes, or hypoxia-activated transcription. Another mechanism includes c-Met gene structural alterations including point mutations (e.g., in hereditary papillary renal carcinomas, childhood hepatocellular carcinomas, sporadic papillary renal carcinomas, gastric carcinomas and head and neck squamous-cell carcinomas) and chromosomal translocations. Yet another mechanism includes c-Met structural alterations such as abnormal posttranslational processing, lack of cleavage of the precursor protein, mutations that prevent receptor downregulation and truncation of the receptor (e.g., in musculoskeletal tumors). Still another mechanism is HGF-dependent autocrine/paracrine activation. Paracrine activation can become pathological in the presence of abnormal HGF production by mesenchymal cells. Autocrine activation occurs when tumor cells aberrantly expression both c-Met and HGF (e.g., in osteosarcomas, rhabdomyosarcomas, gliomas and carcinomas of the thyroid, breast and lung). Finally, constitutive c-Met activation can also be caused by transactivation by other membrane receptors (e.g., RON, EGF-receptor family members, FAS and B plexins). See Corso et al., TRENDS Mol. Med. 11:284 (2005).

Anti-cancer strategies targeting the c-Met signaling pathway are also discussed in Corso et al., supra. These have included antagonism or neutralization of HGF, inhibition of c-Met kinase activity, prevention of c-Met dimerization, inhibition of c-Met intracellular activities, and silencing of c-Met or Hgf expression. Michielli et al., Cancer Cell, 6: 61-73 (2004) describe a soluble c-Met receptor, termed “decoy Met,” that interferes with both HGF binding to c-Met and c-Met homodimerization. Delivery of the decoy Met by a lentiviral vector in mice was reported to inhibit tumor cell proliferation and survival in human xenografts. Decoy Met was observed to impair tumor angiogenesis, suppress formation of spontaneous metastases, and synergize with radiotherapy in inducing tumor regression.

In some embodiments, binding domains of this disclosure comprise V_{H} and V_{L} domains specific for a HGF. In certain embodiments, the V_{H} and V_{L} domains are rodent (e.g., mouse, rat), humanized, or human. Examples of binding domains containing such V_{H} and V_{L} domains specific for HGF include, but are not limited to, those disclosed in US Patent Application Publication no. US 2005/0118643. Binding domains of this disclosure may also, or alternatively, comprise a cMet ectodomain of SEQ ID NO:750, 751 or 752, or a fragment thereof. In further embodiments, there are provided polypeptide binding domains specific for HGF, wherein the binding domain comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to an amino acid sequence of SEQ ID NO:750, 751 or 752, wherein the polypeptide binding domain binds to HGF and inhibits the activity thereof.

In some embodiments, binding domains of this disclosure are c-Met antagonist domains that comprise V_{H} and V_{L} domains as described herein. In certain embodiments, the V_{H} and V_{L} domains are human. Examples of binding domains containing such V_{H} and V_{L} domains are set forth in SEQ ID NOS:1132-1184 and 1079-1131, respectively. In further embodiments, there are provided c-Met antagonist domains of this disclosure that have a sequence that is at least 90%, at least 91%, at least 92%, at least 93%, at least 94%, at least 95%, at least 96%, at least 97%, at least 98%, at least 99%, at least 99.5%, or at least 100% identical to the amino acid sequence of one or more light chain variable regions (V_{L}) or to one or more heavy chain variable regions (V_{H}), or both, as set forth in SEQ ID NOS:1079-1131 and 1132-1184, respectively, wherein each CDR has at most up to three amino acid changes.

In further embodiments, c-Met antagonist domains of this disclosure comprise V_{H} and V_{L} domains as set forth in SEQ ID NOS:1132-1184 and 1079-1131, respectively, which are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of such V_{H} domain, V_{L} domain, or both, wherein each CDR has no more than zero, one, two, or three mutations. For example, the amino acid sequence of a V_{H} domain, V_{L} domain, or both of this disclosure can be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of V_{H} domain (SEQ ID NO:1174), V_{L} domain (SEQ ID NO:1121), or both, respectively, from exemplary binding domain TRU(H)-343.

A binding domain of this disclosure can comprise a single CDR from a variable region of an anti-HGF or anti-c-Met, or it can comprise multiple CDRs that can be the same or different. In certain embodiments, binding domains of this disclosure comprise V_{H} and V_{L} domains specific for an HGF or c-Met comprising framework regions and CDR1, CDR2 and CDR3 regions, wherein (a) the V_{H} domain comprises an amino acid sequence of a heavy chain CDR3 found in any one of SEQ ID NOS:1132-1184; or (b) the V_{L} domain comprises an amino acid sequence of a light chain CDR3 found in any one of SEQ ID NOS:1079-1131; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for an HGF or c-Met comprising framework regions and CDR1, CDR2 and CDR3 regions, wherein (a) the V_{H} domain comprises an amino acid sequence of a heavy chain CDR1, CDR2, and CDR3 found in any one of SEQ ID NOS:1132-1184; or (b) the V_{L} domain comprises an amino acid sequence of a light chain CDR1, CDR2, and CDR3 found in any one of SEQ ID NOS:1079-1131; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence.

In any of the embodiments described herein comprising specific CDRs, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain found in any one of SEQ ID NOS:1132-1184; or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain found in any one of SEQ ID NOS:1079-1131; or (iii) both a V_{H} domain of (i) and a V_{L} domain of (ii); or both a V_{H} domain of (i) and a V_{L} domain of (ii) wherein the V_{H} and V_{L} are from the same reference sequence. Exemplary light and heavy chain variable domain CDRs directed against c-Met are provided in SEQ ID NO:762-920 and 921-1078, respectively.

Amino acid sequences of c-Met antagonist light chain and heavy chain variable regions are provided in SEQ ID NO:1079-1131 and 1132-1184, respectively.

### TWEAK Antagonists

In certain embodiments the present disclosure provides polypeptides containing a binding region or domain that is a TWEAK antagonist (i.e., can inhibit TWEAKR signaling). Exemplary TWEAK antagonists include binding domains specific for a TWEAK, such as an immunoglobulin variable binding domain or derivative thereof (e.g., an antibody, Fab, scFv, or the like), or a TWEAKR ectodomain or fragment thereof.

TWEAK is a cytokine that belongs to the tumor necrosis factor (TNF) ligand family and regulates multiple cellular responses including pro-inflammatory activity, angiogenesis and cell proliferation. TWEAK is a type II-transmembrane protein that is cleaved to generate a soluble cytokine with biological activity. The position of various domains within the TWEAK protein is shown, for example, in US Published Patent Application No. 2007/0280940. TWEAK has overlapping signaling functions with TNF, but displays a much wider tissue distribution. TWEAK can induce apoptosis via multiple pathways of cell death in a cell type-specific manner and has also been found to promote proliferation and migration of endothelial cells, and thus acts as a regulator of angiogenesis.

The cognate TWEAK receptor, TWEAKR or fibroblast growth factor-inducible 14 (Fn14), is a TNF receptor superfamily member expressed by non-lymphoid cell types (Wiley et al. (2001) Immunity 15:837). Expression of TWEAK and TWEAKR is relatively low in normal tissues but undergoes dramatic upregulation in settings of tissue injury and diseases. The TWEAK/R pathway facilitates acute tissue repair functions and thus functions physiologically after acute injury but functions pathologically in chronic inflammatory disease settings. In contrast to TNF, TWEAK plays no apparent role in development or homeostasis. A review of the TWEAK/R pathway is provided in Burkly et al. (2007) Cytokine 40:1. Persistently activated TWEAK promotes chronic inflammation, pathological hyperplasia and angiogenesis, and potentially impedes tissue repair by inhibiting differentiation of progenitor cells. TWEAK protein has been identified on the surface of activated monocytes and T cells and on tumor cell lines, and intracellularly in resting and activated monocytes, dendritic cells and NK cells. TWEAK expression is significantly increased locally in target tissues in contexts of acute injury, inflammatory disease and cancer, all of which are associated with infiltration of inflammatory cells and/or activation of resident innate immune cell types. Circulating TWEAK levels have been shown to be significantly increased in patients with chronic inflammatory diseases such as multiple sclerosis and systemic lupus erythematosus.

TWEAK blocking monoclonal antibodies have been shown to be effective in a mouse collagen-induced arthritis (CIA) model (Kamata et al. (2006) J. Immunol. 177:6433; Perper et al. (2006) J. Immunol. 177:2610). The arthritogenic activities of TWEAK and TNF on human synoviocytes are often additive or synergistic and appear independent of one another, indicating that TWEAK and TNF may act in parallel in pathology of rheumatoid arthritis. It has been speculated that the heterogeneity of RA patients with respect to their clinical response to TNF inhibitors may reflect a pathological contribution by TWEAK.

U.S. Pat. No. 7,169,387 describes the preparation of a monoclonal antibody specific for TWEAK and its use to block aspects of the development of graft-versus-host disease (GVHD) using a mouse model of chronic GVHD. US Patent Application Publication No. 2007/0280940 describes TWEAKR decoy receptors and antibodies against TWEAKR and TWEAK and their use in the treatment of central nervous system diseases associated with cerebral edema and cell death.

In some embodiments, binding domains of this disclosure comprise V_{H} and V_{L} domains specific for a TWEAK. In certain embodiments, the V_{H} and V_{L} domains are rodent (e.g., mouse, rat), humanized, or human. Examples of binding domains containing such V_{H} and V_{L} domains specific for TWEAK, include those disclosed, for example, in U.S. Pat. No. 7,169,387. Monoclonal antibodies that block TWEAK have been shown to be effective in a mouse collagen-induced arthritis (CIA) model (Kamata et al. (2006) J. Immunol. 177:6433; Perper et al. (2006) J. Immunol. 177:2610).

In certain embodiments, a TWEAK antagonist may be an extracellular domain (“ectodomain”) of a TWEAKR (also known as FN14). As used herein, a TWEAKR ectodomain refers to an extracellular portion of TWEAKR, a soluble TWEAKR, or any combination thereof. In certain embodiments, a TWEAK antagonist comprises an amino-terminal portion of TWEAKR, such as the first 70 amino acids of TWEAKR as set forth in GenBank Accession No. NP_{—}057723.1 (SEQ ID NO:761), or any fragment thereof that continues to function as a TWEAK antagonist. In other embodiments, a TWEAK antagonist comprises amino acids 28-70 of SEQ ID NO:761 (i.e., without the native leader sequence). In yet further embodiments, a TWEAK antagonist comprises a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to an amino acid sequence of SEQ ID NO:761, or amino acids 28-70 of SEQ ID NO:761, wherein the antagonist binds to TWEAK and inhibits the activity thereof.

The ability of binding proteins or fusion proteins described herein to reduce binding of TWEAK to TWEAKR may be determined using assays known to those of skill in the art including those described in US Patent Application Publication No. 2007/0280940.

### IGF Antagonists

As noted above, in certain embodiments the present disclosure provides polypeptides containing a binding region or domain that is an IGF1 or IGF2 antagonist (i.e., can inhibit IGF1 or IGF2 signaling). Exemplary IGF1 or IGF2 antagonists include binding domains specific for IGF1 or IGF2, such as an immunoglobulin variable binding domain or derivative thereof (e.g., an antibody, Fab, scFv, or the like), or an IGF1R or IGFBP ectodomain or sub-domain thereof.

The insulin-like growth factors (IGFs), comprise a family of peptides that play important roles in mammalian growth and development. Insulin-like growth factor 1 (IGF1) is a secreted protein that has the following features: disulfide bonds (amino acids 54-96, 66-109, 95-100); D peptide domain (amino acids 111-118); carboxyl-terminal propeptide domain (E peptide) (amino acids 119-153); insulin chain A-like domain (amino acids 90-110); insulin chain B-like domain (amino acids 49-77); insulin connecting C peptide-like domain (amino acids 78-89); propeptide domain (amino acids 22-48); and signal sequence domain (amino acids 1-21).

IGF1 is synthesized in multiple tissues including liver, skeletal muscle, bone and cartilage. The changes in blood concentrations of IGF1 reflect changes in its synthesis and secretion from the liver, which accounts for 80% of the total serum IGF1 in experimental animals. The remainder of the IGF1 is synthesized in the periphery, usually by connective tissue cell types, such as stromal cells that are present in most tissues. IGF1 that is synthesized in the periphery can function to regulate cell growth by autocrine and paracrine mechanisms. Within these tissues, the newly synthesized and secreted IGF1 can bind to receptors that are present either on the connective tissue cells themselves and stimulate growth (autocrine), or it can bind to receptors on adjacent cell types (often epithelial cell types) that do not actually synthesize IGF1 but are stimulated to grow by locally secreted IGF1 (paracrine) (Clemmons, 2007, Nat Rev Drug Discov. 6(10): 821-33). IGF1 synthesis is controlled by several factors, including the human pituitary growth hormone (GH, also known as somatotropin). IGF2 concentrations are high during fetal growth but are less GH-dependent in adult life compared with IGF1.

IGF1 enhances growth and/or survival of cells in a variety of tissues including musculoskeletal systems, liver, kidney, intestines, nervous system tissues, heart, and lung. IGF1 also has an important role in promoting cell growth and consequently IGF1 inhibition is being pursued as a potential adjunctive measure for treating atherosclerosis. Inhibiting IGF1 action has been proposed as a specific treatment either for potentiating the effects of other forms of anticancer therapies or for directly inhibiting tumor cell growth.

Like IGF1, IGF2 acts through IGF1R. IGF2 is an important autocrine growth factor in tumors due to its mitogenic and antiapoptotic functions (Kaneda et al., 2005, Cancer Res 65(24): 11236-11240). Increased expression of IGF2 is found frequently in a wide variety of malignancies, including colorectal, liver, esophageal and adrenocortical cancer, as well as sarcomas. Paracrine signaling by IGF2 also plays a role in tumors including breast cancers, as abundant expression of IGF2 is found in stromal fibroblasts surrounding malignant breast epithelial cells.

Insulin-like growth factor 1 receptor (IGF1R) is a tetramer of two alpha and two beta chains linked by disulfide bonds. Cleavage of a precursor generates the alpha and beta subunits. IGF1R is related to the protein kinase superfamily, the tyrosine protein kinase family, and the insulin receptor subfamily. It contains three fibronectin type-III domains, and one protein kinase domain (Lawrence et al., 2007, Current Opinion in Structural Biology 17: 699-705). The alpha chains contribute to the formation of the ligand-binding domain, while the beta chain carries a kinase domain. It is a single-pass type I membrane protein and is expressed in a variety of tissues.

The kinase domain has tyrosine-protein kinase activity, which is necessary for the activation of the IGF1- or IGF2-stimulated downstream signaling cascade. Auto-phosphorylation activates the kinase activity. IGF1R interacts with PIK3R1 and with the PTB/PID domains of IRS1 and SHC1 in vitro when autophosphorylated on tyrosine residues in the cytoplasmic domain of the beta subunit. IGF1R plays a critical role in transformation events. It is highly over-expressed in most malignant tissues where it functions as an anti-apoptotic agent by enhancing cell survival. Cells lacking this receptor cannot be transformed by most oncogenes, with the exception of v-Src.

The insulin-like growth factor-binding protein (IGFBP) family comprises six soluble proteins (IGFBP1-6) of approximately 250 residues that bind to IGFs with nanomolar affinities. Because of their sequence homology, IGFBPs are assumed to share a common overall fold and are expected to have closely related IGF-binding determinants. Each IGFBP can be divided into three distinct domains of approximately equal lengths: highly conserved cysteine-rich N and C domains and a central linker domain unique to each IGFBP species. Both the N and C domains participate in the binding to IGFs, although the specific roles of each of these domains in IGF binding have not been decisively determined. The C-terminal domain may be responsible for preferences of IGFBPs for one species of IGF over the other; the C-terminal domain is also involved in regulation of the IGF-binding affinity through interaction with extracellular matrix components and is most probably engaged in mediating IGF1-independent actions. The central linker domain is the least conserved region and has never been cited as part of the IGF-binding site for any IGFBP. This domain is the site of posttranslational modifications, specific proteolysis, and the acid-labile subunit and extracellular matrix associations known for IGFBPs. Proteolytic cleavage in this domain is believed to produce lower-affinity N- and C-terminal fragments that cannot compete with IGF receptors for IGFs, and, thus, the proteolysis is assumed to be the predominant mechanism for IGF release from IGFBPs. However, recent studies indicate that the resulting N- and C-terminal fragments still can inhibit IGF activity and have functional properties that differ from those of the intact proteins (Sitar et al. (2006) Proc. Natl. Acad. Sci. USA. 103(35):13028-33).

IGF-binding proteins are secreted proteins that prolong the half-life of the IGFs and have been shown to either inhibit or stimulate the growth promoting effects of the IGFs on cell culture. They alter the interaction of IGFs with their cell surface receptors and also promote cell migration. They bind equally well to IGF1 and IGF2. The C-terminal domains of all IGFBPs show sequence homology with thyroglobulin type-1 domains and share common elements of secondary structure: an α-helix and a 3- to 4-β-stranded β-sheet. The core of the molecule is connected by the consensus three disulfide pairings, has conserved Tyr/Phe amino acids and has the QC, CWCV motifs. These essential features are preserved in CBP1, CBP4, and CBP-6, the structures of C domains solved so far, although there are significant variations in detail. For example, CBP4 has helix α2, whereas the corresponding residues in CBP1 form a short beta-strand seen in other structures of the thyroglobulin type-1 domain superfamily. This particular region of CBPs has high sequence diversity and is involved in the IGF complex formation and thus may perform the role of an affinity regulator.

Inhibition of IGF/IGF-receptor binding interferes with cell growth and represents a strategy for the development of IGFBPs and variants as natural IGF antagonists in many common diseases that arise from disregulation of the IGF system, including diabetes, atherosclerosis, and cancer.

In some embodiments, binding domains of this disclosure comprise V_{H} and

V_{L} domains specific for IGF1 or IGF2. In certain embodiments, the V_{H} and V_{L} domains are rodent (e.g., mouse, rat), humanized, or human. Binding domains of this disclosure may also, or alternatively, comprise an IGF1R ectodomain of Genbank Accession no. NP_{—}000866.1 (SEQ ID NO:753) or a sub-domain thereof, or an IGFBP ectodomain of Genbank Accession no. NP_{—}000587.1 (IGFBP1; SEQ ID NO:754), NP_{—}000588.2 (IGFBP2; SEQ ID NO:755), NP_{—}001013416.1 (IGFBP3 isoform a; SEQ ID NO:756), NP_{—}000589.2 (IGFBP3 isoform b; SEQ ID NO:757), NP_{—}001543.2 (IGFBP4; SEQ ID NO:758), NP_{—}000590.1 (IGFBP5; SEQ ID NO:759) or NP_{—}002169.1 (IGFBP6; SEQ ID NO:760) or a sub-domain thereof. In yet further embodiments, an IGF1 or IGF2 antagonist comprises a sequence that is at least 80%, at least 85%, at least 90%, at least 91%, at least 92%, at least 93%, at least 94%, at least 95%, at least 96%, at least 97%, at least 98%, at least 99%, at least 99.5% or at least 100% identical to an amino acid sequence of SEQ ID NO:754-760, wherein the antagonist inhibits the activity of at least one or IGF1 and IGF2.

### Multi-Specific Fusion Proteins

The present disclosure provides multi-specific fusion proteins comprising a domain that is an antagonist of TGFβ (“TGFβ antagonist domain”) and a domain that is an antagonist or agonist of a ligand other than a TGFβ ligand (“heterologous binding domain”), such as an IL6 antagonist, IL10 antagonist, GITR agonist, VEGF antagonist, TNF antagonist, HGF antagonist, TWEAK antagonist, or IGF antagonist. It is contemplated that the TGFβ antagonist domain may be at the amino-terminus and the heterologous binding domain at the carboxy-terminus of a fusion protein, or the heterologous binding domain may be at the amino-terminus and the TGFβ antagonist may be at the carboxy-terminus. As set forth herein, the binding domains of this disclosure may be fused to each end of an intervening domain (e.g., an immunoglobulin constant region or sub-region thereof). Furthermore, the two or more binding domains may be each joined to an intervening domain via a linker known in the art or as described herein.

As used herein, an “intervening domain” refers to an amino acid sequence that simply functions as a scaffold for one or more binding domains so that the fusion protein will exist primarily (e.g., 50% or more of a population of fusion proteins) or substantially (e.g., 90% or more of a population of fusion proteins) as a single chain polypeptide in a composition. For example, certain intervening domains can have a structural function (e.g., spacing, flexibility, rigidity) or biological function (e.g., an increased half-life in plasma, such as in human blood). Exemplary intervening domains that can increase half-life of the fusion proteins of this disclosure in plasma include albumin, transferrin, a scaffold domain that binds a serum protein, or the like, or fragments thereof.

In certain preferred embodiments, the intervening domain contained in a multi-specific fusion protein of this disclosure is a “dimerization domain,” which refers to an amino acid sequence that is capable of promoting the association of at least two single chain polypeptides or proteins via non-covalent or covalent interactions, such as by hydrogen bonding, electrostatic interactions, Van der Waal's forces, disulfide bonds, hydrophobic interactions, or the like, or any combination thereof. Exemplary dimerization domains include immunoglobulin heavy chain constant regions or sub-regions. It should be understood that a dimerization domain can promote the formation of dimers or higher order multimer complexes (such as trimers, tetramers, pentamers, hexamers, septamers, octamers, etc.).

A “constant sub-region” is a term defined herein to refer to a peptide, polypeptide, or protein sequence that corresponds to or is derived from part or all of one or more immunoglobulin constant region domains, but does not contain all constant region domains found in a source antibody. In preferred embodiments, the constant region domains of a fusion protein of this disclosure contains a CH2 domain and a CH3 domain of IgG, IgA, or IgD, more preferably IgG1 CH2 and CH3, and even more preferably human IgG1 CH2 and CH3. In some embodiments, the constant region domains of a fusion protein of this disclosure lack or have minimal effector functions of antibody-dependent cell-mediated cytotoxicity (ADCC), antibody-dependent cell-mediated phagocytosis (ADCP), and complement activation and complement-dependent cytotoxicity (CDC), while retaining the ability to bind some F_{C} receptors (such as F_{C}Rn binding) and retaining a relatively long half life in vivo. In certain embodiments, a binding domain of this disclosure is fused to a human IgG1 constant region or sub-region, wherein the IgG1 constant region or sub-region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (EU numbering).

Methods are known in the art for making mutations inside or outside an Fc domain that can alter Fc interactions with Fc receptors (CD16, CD32, CD64, CD89, FcεR1, FcRn) or with the complement component C1q (see, e.g., U.S. Pat. No. 5,624,821; Presta (2002) Curr. Pharma. Biotechnol. 3:237). Particular embodiments of this disclosure include compositions comprising immunoglobulin or fusion proteins that have a constant region or sub-region from human IgG wherein binding to FcRn and protein A are preserved and wherein the Fc domain no longer interacts or minimally interacts with other Fc receptors or C1q. For example, a binding domain of this disclosure can be fused to a human IgG1 constant region or sub-region wherein the asparagine at position 297 (N297 under EU numbering) has been mutated to another amino acid to reduce or eliminate glycosylation at this site and, therefore, abrogate efficient Fc binding to FcγR and C1q. Another exemplary mutation is a P331S, which knocks out C1q binding but does not affect Fc binding.

In further embodiments, an immunoglobulin Fc region may have an altered glycosylation pattern relative to an immunoglobulin referent sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831). Alternatively, the host cells in which fusion proteins of this disclosure are produced may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of fusion proteins of this disclosure. For example, a variety of glycosidase and/or mannosidase inhibitors provide one or more of desired effects of increasing ADCC activity, increasing Fc receptor binding, and altering glycosylation pattern. In certain embodiment, cells expressing a multispecific fusion protein of the instant disclosure (containing a TGFβ antagonist domain linked to a IL6, IL6R, IL6xR, IL10, VEGF, TNF, HGF, TWEAK, IGF antagonist or to a GITR agonist) are grown in a culture medium comprising a carbohydrate modifier at a concentration that increases the ADCC of immunoglycoprotein molecules produced by said host cell, wherein said carbohydrate modifier is at a concentration of less than 800 μM. In a preferred embodiment, the cells expressing these multispecific fusion proteins are grown in a culture medium comprising castanospermine or kifunensine, more preferably castanospermine at a concentration of 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in US Patent Application Publication No. 2009/0041756 or PCT Publication No. WO 2008/052030.

In another embodiment, the immunoglobulin Fc region may have amino acid modifications that affect binding to effector cell Fc receptors. These modifications can be made using any technique known in the art, such as the approach disclosed in Presta et al. (2001) Biochem. Soc. Trans. 30:487. In another approach, the Xencor XmAb technology is available to engineer constant sub-regions corresponding to Fc domains to enhance cell killing effector function (see Lazar et al. (2006) Proc. Nat'l. Acad. Sci. (USA) 103:4005). Using this approach, for example, one can generate constant sub-regions with improved specificity and binding for FCγR, thereby enhancing cell killing effector function.

In still further embodiments, a constant region or sub-region can optionally increase plasma half-life or placental transfer in comparison to a corresponding fusion protein lacking such an intervening domain. In certain embodiments, the extended plasma half-life of a fusion protein of this disclosure is at least two, at least three, at least four, at least five, at least ten, at least 12, at least 18, at least 20, at least 24, at least 30, at least 36, at least 40, at least 48 hours, at least several days, at least a week, at least two weeks, at least several weeks, at least a month, at least two months, at least several months, or more in a human.

A constant sub-region may include part or all of any of the following domains: a C_{H2} domain, a C_{H3} domain (IgA, IgD, IgG, IgE, or IgM), and a C_{H4} domain (IgE or IgM). A constant sub-region as defined herein, therefore, can refer to a polypeptide that corresponds to a portion of an immunoglobulin constant region. The constant sub-region may comprise a C_{H2} domain and a C_{H3} domain derived from the same, or different, immunoglobulins, antibody isotypes, or allelic variants. In some embodiments, the C_{H3} domain is truncated and comprises a carboxy-terminal sequence listed in PCT Publication No. WO 2007/146968 as SEQ ID NO:366-371, which sequences are hereby incorporated by reference. In certain embodiments, a constant sub-region of a polypeptide of this disclosure has a C_{H2} domain and C_{H3} domain, which may optionally have an amino-terminal linker, a carboxy-terminal linker, or a linker at both ends.

A “linker” is a peptide that joins or links other peptides or polypeptides, such as a linker of about 2 to about 150 amino acids. In fusion proteins of this disclosure, a linker can join an intervening domain (e.g., an immunoglobulin-derived constant sub-region) to a binding domain or a linker can join two variable regions of a binding domain. For example, a linker can be an amino acid sequence obtained, derived, or designed from an antibody hinge region sequence, a sequence linking a binding domain to a receptor, or a sequence linking a binding domain to a cell surface transmembrane region or membrane anchor. In some embodiments, a linker can have at least one cysteine capable of participating in at least one disulfide bond under physiological conditions or other standard peptide conditions (e.g., peptide purification conditions, conditions for peptide storage). In certain embodiments, a linker corresponding or similar to an immunoglobulin hinge peptide retains a cysteine that corresponds to the hinge cysteine disposed toward the amino-terminus of that hinge. In further embodiments, a linker is from an IgG1 or IgG2A hinge and has one cysteine or two cysteines corresponding to hinge cysteines. In certain embodiments, one or more disulfide bonds are formed as inter-chain disulfide bonds between intervening domains. In other embodiments, fusion proteins of this disclosure can have an intervening domain fused directly to a binding domain (i.e., absent a linker or hinge). In some embodiments, the intervening domain is a dimerization domain.

The intervening or dimerization domain of multi-specific fusion proteins of this disclosure may be connected to one or more terminal binding domains by a peptide linker. In addition to providing a spacing function, a linker can provide flexibility or rigidity suitable for properly orienting the one or more binding domains of a fusion protein, both within the fusion protein and between or among the fusion proteins and their target(s). Further, a linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a linker of an intervening or a dimerization domain of multi-specific fusion proteins of this disclosure may comprise part or all of a human immunoglobulin hinge.

Additionally, a binding domain may comprise a V_{H} and a V_{L} domain, and these variable region domains may be combined by a linker. Exemplary variable region binding domain linkers include those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5 (see, e.g., Linkers 22, 29, 46, 89, 90, and 116 corresponding to SEQ ID NOS:518, 525, 542, 585, 586 and 603, respectively). In preferred embodiments, these (Gly₄Ser)-based linkers are used to link variable domains and are not used to link a binding domain (e.g., scFv) to an intervening domain (e.g., an IgG CH₂CH₃).

Exemplary linkers that can be used join an intervening domain (e.g., an immunoglobulin-derived constant sub-region) to a binding domain or to join two variable regions of a binding domain are provided in SEQ ID NO:497-604 and 1223-1228.

Linkers contemplated in this disclosure include, for example, peptides derived from any inter-domain region of an immunoglobulin superfamily member (e.g., an antibody hinge region) or a stalk region of C-type lectins, a family of type II membrane proteins. These linkers range in length from about two to about 150 amino acids, or about two to about 40 amino acids, or about eight to about 20 amino acids, preferably about ten to about 60 amino acids, more preferably about 10 to about 30 amino acids, and most preferably about 15 to about 25 amino acids. For example, Linker 1 (SEQ ID NO:497) is two amino acids in length and Linker 116 (SEQ ID NO:603) is 36 amino acids in length.

Beyond general length considerations, a linker suitable for use in the fusion proteins of this disclosure includes an antibody hinge region selected from an IgG hinge, IgA hinge, IgD hinge, IgE hinge, or variants thereof. In certain embodiments, a linker may be an antibody hinge region (upper and core region) selected from human IgG1, human IgG2, human IgG3, human IgG4, or fragments or variants thereof. As used herein, a linker that is an “immunoglobulin hinge region” refers to the amino acids found between the carboxyl end of CH1 and the amino terminal end of CH2 (for IgG, IgA, and IgD) or the amino terminal end of CH3 (for IgE and IgM). A “wild type immunoglobulin hinge region,” as used herein, refers to a naturally occurring amino acid sequence interposed between and connecting the CH1 and CH2 regions (for IgG, IgA, and IgD) or interposed between and connecting the CH2 and CH3 regions (for IgE and IgM) found in the heavy chain of an antibody. In preferred embodiments, the wild type immunoglobulin hinge region sequences are human.

According to crystallographic studies, an IgG hinge domain can be functionally and structurally subdivided into three regions: the upper hinge region, the core or middle hinge region, and the lower hinge region (Shin et al. (1992) Immunological Reviews 130:87). Exemplary upper hinge regions include EPKSCDKTHT (SEQ ID NO:1240) as found in IgG1, ERKCCVE (SEQ ID NO:1241) as found in IgG2, ELKTPLGDTT HT (SEQ ID NO:1242) or EPKSCDTPPP (SEQ ID NO:1243) as found in IgG3, and ESKYGPP (SEQ ID NO:1244) as found in IgG4. Exemplary middle hinge regions include CPPCP (SEQ ID NO:1245) as found in IgG1 and IgG2, CPRCP (SEQ ID NO:1246) as found in IgG3, and CPSCP (SEQ ID NO:1247) as found in IgG4. While IgG1, IgG2, and IgG4 antibodies each appear to have a single upper and middle hinge, IgG3 has four in tandem—one of ELKTPLGDTT HTCPRCP (SEQ ID NO:1248) and three of EPKSCDTPPP CPRCP (SEQ ID NO:1249).

IgA and IgD antibodies appear to lack an IgG-like core region, and IgD appears to have two upper hinge regions in tandem (see SEQ ID NOS:1250 and 1251). Exemplary wild type upper hinge regions found in IgA1 and IgA2 antibodies are set forth in SEQ ID NOS:1252 and 1253.

IgE and IgM antibodies, in contrast, instead of a typical hinge region have a CH2 region with hinge-like properties. Exemplary wild-type CH2 upper hinge-like sequences of IgE and IgM are set forth in SEQ ID NO:1254 (VCSRDFTPPT VKILQSSSDG GGHFPPTIQL LCLVSGYTPG TINITWLEDG QVMDVDLSTA STTQEGELAS TQSELTLSQK HWLSDRTYTC QVTYQGHTFE DSTKKCA) and SEQ ID NO:1255 (VIAELPPKVS VFVPPRDGFF GNPRKSKLIC QATGFSPRQI QVSWLREGKQ VGSGVTTDQV QAEAKESGPT TYKVTSTLTI KESDWLGQSM FTCRVDHRGL TFQQNASSMC VP), respectively.

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), (b) a portion of a wild type immunoglobulin hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a wild type immunoglobulin hinge region that comprises the core hinge region (which portion may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild type immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from five to 60 amino acids long, and may be primarily flexible, but may also provide more rigid characteristics, and may contain primarily an α-helical structure with minimal β-sheet structure. Preferably, sequences are stable in plasma and serum and are resistant to proteolytic cleavage. In some embodiments, sequences may contain a naturally occurring or added motif such as CPPC that confers the capacity to form a disulfide bond or multiple disulfide bonds to stabilize the C-terminus of the molecule. In other embodiments, sequences may contain one or more glycosylation sites. Examples of hinge and linker sequences include interdomain regions between the IgV-like and IgC-like or between the IgC-like or IgV-like domains of CD2, CD4, CD22, CD33, CD48, CD58, CD66, CD80, CD86, CD96, CD150, CD166, and CD244. Alternative hinges may also be crafted from disulfide-containing regions of Type II receptors from non-immunoglobulin superfamily members such as CD69, CD72, and CD161.

In some embodiments, a hinge linker has a single cysteine residue for formation of an interchain disulfide bond. In other embodiments, a linker has two cysteine residues for formation of interchain disulfide bonds. In further embodiments, a hinge linker is derived from an immunoglobulin interdomain region (e.g., an antibody hinge region comprising an upper and core sequence of, for example, an IgG1 hinge) or a Type II C-type lectin stalk region (derived from a Type II membrane protein; see, e.g., exemplary lectin stalk region sequences set forth in of PCT Application Publication No. WO 2007/146968, such as SEQ ID NOS:111, 113, 115, 117, 119, 121, 123, 125, 127, 129, 131, 133, 135, 149, 151, 153, 155, 157, 159, 161, 163, 165, 167, 169, 231, 233, 235, 237, 239, 241, 243, 245, 247, 249, 251, 253, 255, 257, 259, 261, 263, 265, 267, 269, 271, 273, 275, 277, 279, 281, 287, 289, 297, 305, 307, 309-311, 313-331, 346, 373-377, 380, or 381 from that publication), which sequences are herein incorporated by reference.

In one aspect, exemplary multi-specific fusion proteins containing a TGFβ antagonist as described herein will also contain at least one additional binding region or domain that is specific for a target other than TGFβ, such as an IL6, IL10, VEGF, TNF, HGF, TWEAK, IGF antagonist or a GITR agonist. For example, a multi-specific fusion protein of this disclosure has a TGFβ antagonist domain linked to an IL6, IL10, VEGF, TNF, HGF, TWEAK, IGF antagonist or a GITR agonist domain by an intervening domain (such as a human IgG1 CH2CH3 Fc region). In certain embodiments, a multi-specific fusion protein comprises a first and second binding domain, a first and second linker, and an intervening domain, wherein one end of the intervening domain is fused via the first linker to a first binding domain that is a TGFβ antagonist (e.g., a TGFβR2 ectodomain, an anti-TGFβR2 ectodomain, an anti-TGFβ) and at the other end is fused via the second linker to a different binding domain that is an IL6, IL10, VEGF, TNF, HGF, TWEAK, IGF antagonist or a GITR agonist.

In certain embodiments, the first linker and second linker of a multi-specific fusion protein of this disclosure are each independently selected from, for example, SEQ ID NO:497-604 and 1223-1228. For example, the first or second linker can be Linker 102 (SEQ ID NO:589), 47 (SEQ ID NO:543), 80 (SEQ ID NO:576), or any combination thereof. In further examples, one linker is Linker 102 (SEQ ID NO:589) and the other linker is Linker 47 (SEQ ID NO:543), or one linker is Linker 102 (SEQ ID NO:589) and the other linker is Linker 80 (SEQ ID NO:576). In further examples, binding domains of this disclosure that comprise V_{H} and V_{L} domains, such as those specific for IL6, IL6R, IL6xR, IL10, VEGF, TNF, HGF, TWEAK, IGF, GITR, TGFβR2 ectodomain, or TGFβ, can have a further (third) linker between the V_{H} and V_{L} domains, such as Linker 46 (SEQ ID NO:542). In any of these embodiments, the linkers may be flanked by one to five additional junction amino acids, which may simply be a result of creating such a recombinant molecule (e.g., use of a particular restriction enzyme site to join nucleic acid molecules may result in the insertion of one to several amino acids), or for purposes of this disclosure may be considered a part of any particular linker core sequence.

In further embodiments, the intervening domain of a multi-specific fusion protein of this disclosure is comprised of an immunoglobulin constant region or sub-region (preferably CH2CH3 of IgG, IgA, or IgD; or CH3CH4 of IgE or IgM), wherein the intervening domain is disposed between a TGFβ antagonist domain and an IL6, IL10, VEGF, TNF, HGF, TWEAK, IGF antagonist binding domain or a GITR agonist binding domain. In certain embodiments, the intervening domain of a multi-specific fusion protein of this disclosure has a TGFβ antagonist at the amino-terminus and a binding domain specific for an IL6, IL6xR, IL10, VEGF, TNF, HGF, TWEAK, IGF, or GITR at the carboxy-terminus. In other embodiments, the intervening domain of a multi-specific fusion protein of this disclosure has a binding domain specific for an IL6, IL10, VEGF, TNF, HGF, TWEAK, IGF antagonist binding domain or a GITR agonist binding domain at the amino-terminus and a TGFβ antagonist at the carboxy-terminus. In further embodiments, the immunoglobulin constant region sub-region includes CH2 and CH3 domains of immunoglobulin G1 (IgG1). In related embodiments, the IgG1 CH2 and CH3 domains have one or more of the following amino acids mutated (i.e., have a different amino acid at that position): leucine at position 234 (L234), leucine at position 235 (L235), glycine at position 237 (G237), glutamate at position 318 (E318), lysine at position 320 (K320), lysine at position 322 (K322), or any combination thereof (EU numbering). For example, any one of these amino acids can be changed to alanine. In a further embodiment, according to Kabat numbering, the CH2 domain has each of L234, L235, G237, E318, K320 and K322 mutated to an alanine (i.e., L234A, L235A, G237A, E318A, K320A and K322A, respectively).

In some embodiments, a multi-specific fusion protein of this disclosure has a TGFβ antagonist that comprises a TGFβR2 ectodomain or a sub-domain of a TGFβR2 ectodomain, or any combination thereof. For example, a TGFβ antagonist can comprise amino acids 73-176 as set forth in GenBank Accession No. NP_{—}001020018.1, amino acids 48-151 as set forth in GenBank Accession No. NP_{—}003233.4, or any combination thereof. In further embodiments, the TGFβ antagonist comprises an amino acid sequence as set forth in SEQ ID NO:743 or 744.

In further embodiments, a multi-specific fusion protein of this disclosure having a TGFβ antagonist of this disclosure also has an IL6 antagonist binding domain that binds with higher affinity to IL6xR than to either IL6 or IL6Rα alone and competes with sIL6xR complex binding to mgp130 or enhances sgp103 binding to sIL6xR complex. In certain embodiments, a binding domain specific for an IL6xR comprises (i) a V_{H} domain having an amino acid sequence that is at least 80%, 85%, 90%, 91%, 92%, 93%, 94%, 95%, 96%, 97%, 98%, 99% or 100% identical to the amino acid sequence of a V_{H} domain found in any one of SEQ ID NOS:435-496; or (ii) a V_{L} domain having an amino acid sequence that is at least 80%, 85%, 90%, 91%, 92%, 93%, 94%, 95%, 96%, 97%, 98%, 99% or 100% identical to the amino acid sequence of a V_{L} domain found in any one of SEQ ID NOS:373-434; or (iii) both a V_{H} domain of (i) and a V_{L} domain of (ii); or both a V_{H} domain of (i) and a V_{L} domain of (ii) wherein the V_{H} and V_{L} are from the same reference sequence. In one embodiment, such V_{H} and V_{L} domains can form exemplary binding domain TRUE-1019 (see SEQ ID NOS:453 and 391, respectively).

In still further embodiments, an IL6 antagonist binding domain, which binds to the IL6xR with a higher affinity than IL6 or IL6Rα or either IL6 or IL6Rα alone, and competes with gp130 for binding to the sIL6xR complex or enhances sgp130 binding to sIL6xR complex, comprises V_{H} and V_{L} domains comprising framework regions and CDR1, CDR2 and CDR3 regions, wherein (a) the V_{H} domain comprises the amino acid sequence of a heavy chain CDR1, CDR2, and CDR3 found in any one of SEQ ID NOS:435-496; or (b) the V_{L} domain comprises the amino acid sequence of a light chain CDR1, CDR2, and CDR3 found in any one of SEQ ID NOS:373-434; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence. The V_{L} and V_{H} domains of these multi-specific fusion proteins may be arranged in either orientation and may be separated by up to about a 5-30 amino acid linker as disclosed herein. In certain embodiments, a linker joining the V_{H} and V_{L} domains comprises an amino acid sequence of Linker 47 (SEQ ID NO:543) or Linker 80 (SEQ ID NO:576). In certain embodiments, a multi-specific fusion protein comprising the IL6 antagonist binding domain measurably inhibits IL6 cis- and trans-signaling, preferably trans-signaling and, optionally, does not inhibit signaling of gp130 family cytokines other than IL6.

Exemplary structures of such multi-specific fusion proteins, referred to herein as Xceptor molecules, include N-BD-X-ED-C, N-ED-X-BD-C, N-ED1-X-ED2-C, wherein BD is an immunoglobulin-like or immunoglobulin variable region binding domain, X is an intervening domain, and ED is a receptor ectodomain, or the like. In some constructs, X can comprise an immunoglobulin constant region or sub-region disposed between the first and second binding domains. In some embodiments, a multi-specific fusion protein of this disclosure has an intervening domain (X) comprising, from amino-terminus to carboxy-terminus, a structure as follows: -L1-X-L2-, wherein L1 and L2 are each independently a linker comprising from two to about 150 amino acids; and X is an immunoglobulin constant region or sub-region. In further embodiments, the multi-specific fusion protein will have an intervening domain that is albumin, transferrin, or another serum protein binding protein, wherein the fusion protein remains primarily or substantially as a single chain polypeptide in a composition. In still further embodiments, a multi-specific fusion protein of this disclosure has the following structure: N-BD1-X-L2-BD2-C, wherein N and C represent the amino-terminus and carboxy-terminus, respectively; BD1 is a TGFβ antagonist that is at least about 90% identical to an ectodomain of TGFβ2; —X— is -L1-CH2CH3-, wherein L1 is the first IgG1 hinge, optionally mutated by substituting the first cysteine and wherein —CH2CH3- is the CH2CH3 region of an IgG1 Fc domain, optionally mutated to eliminate FcγRI-III interaction while retaining FcRn interaction; L2 is a linker selected from SEQ ID NO:497-604 and 1223-1228; and BD2 is a binding domain specific for an IL6 or IL6/IL6R complex.

In particular embodiments, a multi-specific Xceptor fusion protein has (a) a TGFβ antagonist comprising an amino acid sequence at least 80% to 100% identical to a sequence as set forth in SEQ ID NO:743 or 744 and (b) an IL6 antagonist comprising a heavy chain variable region with CDR1, CD2, and CDR3 amino acid sequences at least 80% to 100% identical to sequences set forth in SEQ ID NOS:435-496, respectively, and a light chain variable region with CDR1, CDR2, and CDR3 amino acid sequences at least 80% to 100% identical to sequences set forth in SEQ ID NOS:373-434, respectively, wherein, from amino-terminus to carboxy-terminus or from carboxy-terminus to amino-terminus, (i) a TGFβ antagonist of (a) or an IL6 antagonist of (b) is fused to a first linker, (ii) the first linker is fused to an immunoglobulin heavy chain constant region of CH2 and CH3 comprising amino acids 276 to 489 of SEQ ID NO:625, (iii) the CH2CH3 constant region polypeptide is fused to a second linker, and (iv) the second linker is fused to a TGFβ antagonist of (a) or an IL6 antagonist of (b). In certain embodiments, the first linker is Linker 47 (SEQ ID NO:543) or Linker 80 (SEQ ID NO:576), the second linker is Linker 102 (SEQ ID NO:589), and a further (third) linker between the IL6 antagonist V_{H} and V_{L} domains is Linker 46 (SEQ ID NO:542).

In other embodiments, a multi-specific Xceptor fusion protein has (a) a TGFβ antagonist comprising an amino acid sequence at least 80% to 100% identical to a sequence as set forth in SEQ ID NO:743 or 744 and (b) an IL10 antagonist comprising an amino acid sequence at least 80% to 100% identical to an amino acid sequence of SEQ ID NO:745 or to amino acids 22-401 of SEQ ID NO:745, wherein, from amino-terminus to carboxy-terminus or from carboxy-terminus to amino-terminus, (i) a TGFβ antagonist of (a) or an IL10 antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or an IL10 antagonist of (b). In certain embodiments, the first linker is Linker 47 (SEQ ID NO:543) or Linker 80 (SEQ ID NO:576), and the second linker is Linker 102 (SEQ ID NO:589).

In further embodiments, a multi-specific Xceptor fusion protein has (a) a TGFβ antagonist comprising an amino acid sequence at least 80% to 100% identical to a sequence as set forth in SEQ ID NO:743 or 744 and (b) a VEGF antagonist comprising an amino acid sequence at least 80% to 100% identical to an amino acid sequence of SEQ ID NO:747, wherein, from amino-terminus to carboxy-terminus or from carboxy-terminus to amino-terminus, (i) a TGFβ antagonist of (a) or a VEGF antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or a VEGF antagonist of (b). In certain embodiments, the first linker is Linker 47 (SEQ ID NO:543) or Linker 80 (SEQ ID NO:576) and the second linker is Linker 102 (SEQ ID NO:589.

In further embodiments, a multi-specific Xceptor fusion protein has (a) a TGFβ antagonist comprising an amino acid sequence at least 80% to 100% identical to a sequence as set forth in SEQ ID NO:743 or 744 and (b) a TNFα antagonist comprising an amino acid sequence at least 80% to 100% identical to an amino acid sequence of SEQ ID NO:748 or 749, wherein, from amino-terminus to carboxy-terminus or from carboxy-terminus to amino-terminus, (i) a TGFβ antagonist of (a) or a TNFα antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or a TNFα antagonist of (b). In certain embodiments, the first linker is Linker 47 (SEQ ID NO:543) or Linker 80 (SEQ ID NO:576), and the second linker is Linker 102 (SEQ ID NO:589). In specific embodiments, the multi-specific Xceptor fusion protein has an amino acid sequence of SEQ ID NO:1236.

In further embodiments, a multi-specific Xceptor fusion protein has (a) a TGFβ antagonist comprising an amino acid sequence at least 80% to 100% identical to a sequence as set forth in SEQ ID NO:743 or 744 and (b) a HGF antagonist comprising a heavy chain variable region with CDR1, CD2, and CDR3 amino acid sequences at least 80% to 100% identical to sequences set forth in SEQ ID NOS:921-1078, respectively, and a light chain variable region with CDR1, CDR2, and CDR3 amino acid sequences at least 80% to 100% identical to sequences set forth in SEQ ID NOS:762-920, respectively, wherein, from amino-terminus to carboxy-terminus or from carboxy-terminus to amino-terminus, (i) a TGFβ antagonist of (a) or a HGF antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or a HGF antagonist of (b). In certain embodiments, the first linker is Linker 47 (SEQ ID NO:543) or Linker 80 (SEQ ID NO:576), the second linker is Linker 102 (SEQ ID NO:589), and a further (third) linker between the HGF antagonist V_{H} and V_{L} domains is Linker 46 (SEQ ID NO:542).

In yet other embodiments, a multi-specific Xceptor fusion protein has (a) a TGFβ antagonist comprising an amino acid sequence at least 80% to 100% identical to a sequence as set forth in SEQ ID NO:743 or 744 and (b) a TWEAK antagonist comprising an amino acid sequence at least 80% to 100% identical to an amino acid sequence of SEQ ID NO:761, wherein, from amino-terminus to carboxy-terminus or from carboxy-terminus to amino-terminus, (i) a TGFβ antagonist of (a) or a TWEAK antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or a TWEAK antagonist of (b). In certain embodiments, the first linker is Linker 47 (SEQ ID NO:543) or Linker 80 (SEQ ID NO:576), and the second linker is Linker 102 (SEQ ID NO:589). In specific embodiments, the multi-specific Xceptor fusion protein has an amino acid sequence of SEQ ID NO:1237.

In yet other embodiments, a multi-specific Xceptor fusion protein has (a) a TGFβ antagonist comprising an amino acid sequence at least 80% to 100% identical to a sequence as set forth in SEQ ID NO:743 or 744 and (b) an IGF antagonist comprising an amino acid sequence at least 80% to 100% identical to an amino acid sequence of SEQ ID NO:754-760, wherein, from amino-terminus to carboxy-terminus or from carboxy-terminus to amino-terminus, (i) a TGFβ antagonist of (a) or an IGF antagonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or an IGF antagonist of (b). In certain embodiments, the first linker is Linker 47 (SEQ ID NO:543) or Linker 80 (SEQ ID NO:576), and the second linker is Linker 102 (SEQ ID NO:589).

In other embodiments, a multi-specific Xceptor fusion protein has (a) a TGFβ antagonist comprising an amino acid sequence at least 80% to 100% identical to a sequence as set forth in SEQ ID NO:743 or 744 and (b) a GITR agonist comprising an amino acid sequence at least 80% to 100% identical to amino acids 74-181 of SEQ ID NO:746, wherein, from amino-terminus to carboxy-terminus or from carboxy-terminus to amino-terminus, (i) a TGFβ antagonist of (a) or a GITR agonist of (b) is fused to a first linker, (ii) the first linker is fused to an immunoglobulin heavy chain constant region of CH2 and CH3, (iii) the CH2CH3 constant region polypeptide is fused to a second linker, and (iv) the second linker is fused to a TGFβ antagonist of (a) or a GITR agonist of (b). In certain embodiments, the first linker is Linker 47 (SEQ ID NO:543) or Linker 80 (SEQ ID NO:576), and the second linker is Linker 102 (SEQ ID NO:589).

### Making Multi-Specific Fusion Proteins

To efficiently produce any of the binding domain polypeptides or fusion proteins described herein, a leader peptide is used to facilitate secretion of expressed polypeptides and fusion proteins. Using any of the conventional leader peptides (signal sequences) is expected to direct nascently expressed polypeptides or fusion proteins into a secretory pathway and to result in cleavage of the leader peptide from the mature polypeptide or fusion protein at or near the junction between the leader peptide and the polypeptide or fusion protein. A particular leader peptide will be chosen based on considerations known in the art, such as using sequences encoded by polynucleotides that allow the easy inclusion of restriction endonuclease cleavage sites at the beginning or end of the coding sequence for the leader peptide to facilitate molecular engineering, provided that such introduced sequences specify amino acids that either do not interfere unacceptably with any desired processing of the leader peptide from the nascently expressed protein or do not interfere unacceptably with any desired function of a polypeptide or fusion protein molecule if the leader peptide is not cleaved during maturation of the polypeptides or fusion proteins. Exemplary leader peptides of this disclosure include natural leader sequences (i.e., those expressed with the native protein) or use of heterologous leader sequences, such as H₃N-MDFQVQIFSFLLISASVIMSRG(X)ₙ—CO₂H, wherein X is any amino acid and n is zero to three (SEQ ID NO:1185) or H₃N-MEAPAQLLFLLLLWLPDTTG-CO₂H (SEQ ID NO:1186).

As noted herein, variants and derivatives of binding domains, such as ectodomains, light and heavy variable regions, and CDRs described herein, are contemplated. In one example, insertion variants are provided wherein one or more amino acid residues supplement a specific binding agent amino acid sequence. Insertions may be located at either or both termini of the protein, or may be positioned within internal regions of the specific binding agent amino acid sequence. Variant products of this disclosure also include mature specific binding agent products, i.e., specific binding agent products wherein a leader or signal sequence is removed, and the resulting protein having additional amino terminal residues. The additional amino terminal residues may be derived from another protein, or may include one or more residues that are not identifiable as being derived from a specific protein. Polypeptides with an additional methionine residue at position −1 are contemplated, as are polypeptides of this disclosure with additional methionine and lysine residues at positions −2 and −1. Variants having additional Met, Met-Lys, or Lys residues (or one or more basic residues in general) are particularly useful for enhanced recombinant protein production in bacterial host cells.

As used herein, “amino acids” refer to a natural (those occurring in nature) amino acid, a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (GIy or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration.

Substitution variants include those fusion proteins wherein one or more amino acid residues in an amino acid sequence are removed and replaced with alternative residues. In some embodiments, the substitutions are conservative in nature; however, this disclosure embraces substitutions that are also non-conservative. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 1 (see WO 97/09433, page 10, published Mar. 13, 1997), immediately below.

TABLE 1
Conservative Substitutions I

| Side Chain | Characteristic | Amino Acid |
|---|---|---|
| Aliphatic | Non-polar | G, A, P, I, L, V |
| | Polar - uncharged | S, T, M, N, Q |
| | Polar - charged | D, E, K, R |
| Aromatic | | H, F, W, Y |
| Other | | N, Q, D, E |

Alternatively, conservative amino acids can be grouped as described in Lehninger (Biochemistry, Second Edition; Worth Publishers, Inc. NY:NY (1975), pp. 71-77) as set out in Table 2, immediately below.

TABLE 2
Conservative Substitutions II

| Side Chain | Characteristic | Amino Acid |
|---|---|---|
| Non-polar (hydrophobic) | Aliphatic: | A, L, I, V, P |
| | Aromatic | F, W |
| | Sulfur-containing | M |
| | Borderline | G |
| Uncharged-polar | Hydroxyl | S, T, Y |
| | Amides | N, Q |
| | Sulfhydryl | C |
| | Borderline | G |
| Positively Charged (Basic) | | K, R, H |
| Negatively Charged (Acidic) | | D, E |

Variants or derivatives can also have additional amino acid residues which arise from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants which result from expression in other vector systems are also contemplated, including those wherein histidine tags are incorporated into the amino acid sequence, generally at the carboxy and/or amino terminus of the sequence.

Deletion variants are also contemplated wherein one or more amino acid residues in a binding domain of this disclosure are removed. Deletions can be effected at one or both termini of the fusion protein, or from removal of one or more residues within the amino acid sequence.

In certain illustrative embodiments, fusion proteins of this disclosure are glycosylated, the pattern of glycosylation being dependent upon a variety of factors including the host cell in which the protein is expressed (if prepared in recombinant host cells) and the culture conditions.

This disclosure also provides derivatives of fusion proteins. Derivatives include specific binding domain polypeptides bearing modifications other than insertion, deletion, or substitution of amino acid residues. In certain embodiments, the modifications are covalent in nature, and include for example, chemical bonding with polymers, lipids, other organic, and inorganic moieties. Derivatives of this disclosure may be prepared to increase circulating half-life of a specific binding domain polypeptide, or may be designed to improve targeting capacity for the polypeptide to desired cells, tissues, or organs.

This disclosure further embraces fusion proteins that are covalently modified or derivatized to include one or more water-soluble polymer attachments such as polyethylene glycol, polyoxyethylene glycol, or polypropylene glycol, as described U.S. Pat. Nos. 4,640,835; 4,496,689; 4,301,144; 4,670,417; 4,791,192 and 4,179,337. Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)—derivatized proteins. Water-soluble polymers may be bonded at specific positions, for example at the amino terminus of the proteins and polypeptides according to this disclosure, or randomly attached to one or more side chains of the polypeptide. The use of PEG for improving therapeutic capacities is described in U.S. Pat. No. 6,133,426.

A particular embodiment of this disclosure is an immunoglobulin or an Fc fusion protein. Such a fusion protein can have a long half-life, e.g., several hours, a day or more, or even a week or more, especially if the Fc domain is capable of interacting with FcRn, the neonatal Fc receptor. The binding site for FcRn in an Fc domain is also the site at which the bacterial proteins A and G bind. The tight binding between these proteins can be used as a means to purify antibodies or fusion proteins of this disclosure by, for example, employing protein A or protein G affinity chromatography during protein purification.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography; exclusion chromatography; polyacrylamide gel electrophoresis; and isoelectric focusing. Particularly efficient methods of purifying peptides are fast protein liquid chromatography and HPLC.

Certain aspects of the present disclosure concern the purification, and in particular embodiments, the substantial purification, of a fusion protein. The term “purified fusion protein” as used herein, is intended to refer to a composition, isolatable from other components, wherein the fusion protein is purified to any degree relative to its naturally obtainable state. A purified fusion protein therefore also refers to a fusion protein, free from the environment in which it may naturally occur.

Generally, “purified” will refer to a fusion protein composition that has been subjected to fractionation to remove various other components, and which composition substantially retains its expressed biological activity. Where the term “substantially purified” is used, this designation refers to a fusion binding protein composition in which the fusion protein forms the major component of the composition, such as constituting about 50%, about 60%, about 70%, about 80%, about 90%, about 95%, about 99% or more of the protein, by weight, in the composition.

Various methods for quantifying the degree of purification are known to those of skill in the art in light of the present disclosure. These include, for example, determining the specific binding activity of an active fraction, or assessing the amount of fusion protein in a fraction by SDS/PAGE analysis. A preferred method for assessing the purity of a protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed fusion protein exhibits a detectable binding activity.

Various techniques suitable for use in protein purification are well known to those of skill in the art. These include, for example, precipitation with ammonium sulfate, PEG, antibodies and the like, or by heat denaturation, followed by centrifugation; chromatography steps such as ion exchange, gel filtration, reverse phase, hydroxylapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein.

There is no general requirement that the fusion protein always be provided in its most purified state. Indeed, it is contemplated that less substantially purified proteins will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in greater purification than the same technique utilizing a low pressure chromatography system. Methods exhibiting a lower degree of relative purification may have advantages in total recovery of protein product, or in maintaining binding activity of an expressed protein.

It is known that the migration of a polypeptide can vary, sometimes significantly, with different conditions of SDS/PAGE (Capaldi et al. (1977) Biochem. Biophys. Res. Comm. 76:425). It will therefore be appreciated that under differing electrophoresis conditions, the apparent molecular weights of purified or partially purified fusion protein expression products may vary.

### Polynucleotides, Expression Vectors, and Host Cells

This disclosure provides polynucleotides (isolated or purified or pure polynucleotides) encoding the multi-specific fusion protein of this disclosure, vectors (including cloning vectors and expression vectors) comprising such polynucleotides, and cells (e.g., host cells) transformed or transfected with a polynucleotide or vector according to this disclosure.

In certain embodiments, a polynucleotide (DNA or RNA) encoding a binding domain of this disclosure, or a multi-specific fusion protein containing one or more such binding domains is contemplated. Expression cassettes encoding multi-specific fusion protein constructs are provided in the examples appended hereto.

The present disclosure also relates to vectors that include a polynucleotide of this disclosure and, in particular, to recombinant expression constructs. In one embodiment, this disclosure contemplates a vector comprising a polynucleotide encoding a multi-specific fusion protein containing a TGFβ antagonist domain and an IL6 or IL6/IL6R binding domain of this disclosure, along with other polynucleotide sequences that cause or facilitate transcription, translation, and processing of such multi-specific fusion protein-encoding sequences.

Appropriate cloning and expression vectors for use with prokaryotic and eukaryotic hosts are described, for example, in Sambrook et al., Molecular Cloning: A Laboratory Manual, Second Edition, Cold Spring Harbor, N.Y., (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein

As used herein, “vector” means a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences.

In certain embodiments, expression constructs are derived from plasmid vectors. Illustrative constructs include modified pNASS vector (Clontech, Palo Alto, Calif.), which has nucleic acid sequences encoding an ampicillin resistance gene, a polyadenylation signal and a T7 promoter site; pDEF38 and pNEF38 (CMC ICOS Biologics, Inc.), which have a CHEF1 promoter; and pD18 (Lonza), which has a CMV promoter. Other suitable mammalian expression vectors are well known (see, e.g., Ausubel et al., 1995; Sambrook et al., supra; see also, e.g., catalogs from Invitrogen, San Diego, Calif.; Novagen, Madison, Wis.; Pharmacia, Piscataway, N.J.). Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, the fusion protein-encoding nucleic acids as provided herein may be included in any one of a variety of expression vector constructs as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (Current Protocols in Molecular Biology, Greene Publ. Assoc. Inc. & John Wiley & Sons, Inc., Boston, Mass., 1993); Sambrook et al. (Molecular Cloning, Second Ed., Cold Spring Harbor Laboratory, Plainview, N.Y., 1989); Maniatis et al. (Molecular Cloning, Cold Spring Harbor Laboratory, Plainview, N.Y., 1982); Glover (Ed.) (DNA Cloning Vol. I and II, IRL Press, Oxford, UK, 1985); Hames and Higgins (Eds.) (Nucleic Acid Hybridization, IRL Press, Oxford, UK, 1985); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least 90%, and preferably 95%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y., 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

A further aspect of this disclosure provides a host cell transformed or transfected with, or otherwise containing, any of the polynucleotides or vector/expression constructs of this disclosure. The polynucleotides or cloning/expression constructs of this disclosure are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of this disclosure when harboring a polynucleotide, vector, or protein according to this disclosure include, in addition to a subject's own cells (e.g., a human patient's own cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. The selection of an appropriate host is within the scope of those skilled in the art from the teachings herein. Host cells that glycosylate the fusion proteins of this disclosure are contemplated.

The term “recombinant host cell” (or simply “host cell”) refers to a cell containing a recombinant expression vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Recombinant host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman (1981) Cell 23:175, and other cell lines capable of expressing a compatible vector, for example, the C127, 3T3, CHO, HeLa and BHK cell lines. Mammalian expression vectors will comprise an origin of replication, a suitable promoter and, optionally, enhancer, and also any necessary ribosome binding sites, polyadenylation site, splice donor and acceptor sites, transcriptional termination sequences, and 5′-flanking nontranscribed sequences, for example, as described herein regarding the preparation of multivalent binding protein expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including calcium phosphate transfection, DEAE-Dextran-mediated transfection, or electroporation (Davis et al. (1986) Basic Methods in Molecular Biology).

In one embodiment, a host cell is transduced by a recombinant viral construct directing the expression of a protein or polypeptide according to this disclosure. The transduced host cell produces viral particles containing expressed protein or polypeptide derived from portions of a host cell membrane incorporated by the viral particles during viral budding.

### Compositions and Methods of Use

To treat human or non-human mammals suffering a disease state associated with TGFβ, IL6, IL10, GITR, VEGF, TNF, HGF, TWEAK, IGF1 or IGF2 dysregulation, a multi-specific fusion protein of this disclosure is administered to the subject in an amount that is effective to ameliorate symptoms of the disease state following a course of one or more administrations. Being polypeptides, the multi-specific fusion proteins of this disclosure can be suspended or dissolved in a pharmaceutically acceptable diluent, optionally including a stabilizer of other pharmaceutically acceptable excipients, which can be used for intravenous administration by injection or infusion, as more fully discussed below.

A pharmaceutically effective dose is that dose required to prevent, inhibit the occurrence of, or treat (alleviate a symptom to some extent, preferably all symptoms of) a disease state. The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.1 mg/kg and 100 mg/kg body weight (which can be administered as a single dose, or in multiple doses given hourly, daily, weekly, monthly, or any combination thereof that is an appropriate interval) of active ingredient may be administered depending on the potency of a binding domain polypeptide or multi-specific protein fusion of this disclosure.

In certain aspects, compositions of fusion proteins are provided by this disclosure. Pharmaceutical compositions of this disclosure generally comprise one or more type of binding domain or fusion protein in combination with a pharmaceutically acceptable carrier, excipient, or diluent. Such carriers will be nontoxic to recipients at the dosages and concentrations employed. Pharmaceutically acceptable carriers for therapeutic use are well known in the pharmaceutical art, and are described, for example, in Remington's Pharmaceutical Sciences, Mack Publishing Co. (A. R. Gennaro (Ed.) 1985). For example, sterile saline and phosphate buffered saline at physiological pH may be used. Preservatives, stabilizers, dyes and the like may be provided in the pharmaceutical composition. For example, sodium benzoate, sorbic acid, or esters of p-hydroxybenzoic acid may be added as preservatives. Id. at 1449. In addition, antioxidants and suspending agents may be used. Id. The compounds of the present invention may be used in either the free base or salt forms, with both forms being considered as being within the scope of the present invention.

Pharmaceutical compositions may also contain diluents such as buffers; antioxidants such as ascorbic acid, low molecular weight (less than about 10 residues) polypeptides, proteins, amino acids, carbohydrates (e.g., glucose, sucrose, or dextrins), chelating agents (e.g., EDTA), glutathione or other stabilizers or excipients. Neutral buffered saline or saline mixed with nonspecific serum albumin are exemplary appropriate diluents. Preferably, product is formulated as a lyophilizate using appropriate excipient solutions as diluents.

Compositions of this disclosure can be used to treat disease states in human and non-human mammals that are a result of or associated with TGFβ or IL6 dysregulation. Increased production or activity of TGFβ has been implicated in various disease processes, including tumorigenesis, angiogenesis, metastasis, metastatic migration, and epithelial and mesenchymal cancers (see, e.g., Oft et al. (1998) Curr. Biol. 8:1243; Pardali & Mousaka (2007) Biochim. Biophys. Acta 1775:21). In addition, TGFβ signal transduction has been associated with angiogenesis and the development of vascular disorders (Bertolino et al. (2005) Chest 128:585 S).

It has been suggested that IL10 may play a key role in the occurrence of lyphocytic diseases (U.S. Pat. No. 5,639,600) and that IL10 may increase proliferation of non-Hodgkin's lymphoma cells (Voorzanger et al. (1996) Cancer Res. 56:5499). More recently it has been proposed that TGFβ and IL10 work together to ensure a controlled inflammatory response (Li & Flavell, (2008) Immunity 28:468). It has been suggested that tumor-expressed GITRL mediates immunosubversion in humans (Baltz et al. (2007) FASEB J. 21:2442). Overexpression of VEGF and TGFβ has been associated with the development of cervical cancer (Baritaki et al. (2007) Int. J. Oncol. 31:69). TNFα has been associated with the development of renal cell carcinoma (Harrison et al. (2007) J. Clin. Oncol. 25:4542-9). TGFβ has been shown to promote HGF-dependent invasion of squamous carcinoma cells (Lewis et al. (2004) Br. J. Cancer, 90:822), and HGF has been shown to stimulate cell growth and enhance expression of TGFα in human pancreatic cancer cells (Ohba et al. (1999) J. Gastroenterol. 34:498-504). In addition, TGFβ and HGF have been shown to stimulate the invasivenss of gastric cancer cells (Inoue et al., (197) Jpn. J. Cancer Res. 88:152). IGF1R has been identified in the treatment of cancers, including sarcomas (Scotlandi & Picci (2008) Curr. Opin. Oncol. 20:419-27; Yuen & Macaulay (2008) Expert Opin. Ther. Targets 12:589-603).

IL-6 trans-signaling has been implicated in malignancies, such as colon cancer, while IL6 cis-signaling has been implicated in malignancies including hormone-independent prostate cancer, B-cell proliferative disorders such as B cell non-Hodgkin's lymphoma, and advanced cancers of kidney, breast, colon, lung, brain, and other tissues (see, e.g., Sansone et al. (2007) J. Clin Invest. 117:3988). Thus, multi-specific fusion proteins of this disclosure are useful in treating various TGFβ related autoimmune disorders (such as systemic lupus erythematosus (SLE) or rheumatoid arthritis), Alzheimer's disease or hyperproliferative diseases or malignant disorders, including polycystic kidney disease, lung cancer, colon cancer, urothelial cancer, bladder cancer, renal cell cancer, breast cancer, ovarian cancer, Rhabdomyosarcoma, Ewing's sarcoma, osteosarcoma, neuroblastoma, head & neck cancer, melanoma, glioblastoma, pancreatic cancer, or hepatocarcinoma, or the like.

“Pharmaceutically acceptable salt” refers to a salt of a binding domain polypeptide or fusion protein of this disclosure that is pharmaceutically acceptable and that possesses the desired pharmacological activity of the parent compound. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, or the like.

In particular illustrative embodiments, a polypeptide or fusion protein of this disclosure is administered intravenously by, for example, bolus injection or infusion. Routes of administration in addition to intravenous include oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrathecal, intrameatal, intraurethral injection, perispinal or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of this disclosure in aerosol form may hold a plurality of dosage units.

For oral administration, an excipient and/or binder may be present, such as sucrose, kaolin, glycerin, starch dextrans, cyclodextrins, sodium alginate, ethyl cellulose, and carboxy methylcellulose. Sweetening agents, preservatives, dye/colorant, flavor enhancer, or any combination thereof may optionally be present. A coating shell may also optionally be used.

In a composition intended to be administered by injection, one or more of a surfactant, preservative, wetting agent, dispersing agent, suspending agent, buffer, stabilizer, isotonic agent, or any combination thereof may optionally be included.

For nucleic acid-based formulations, or for formulations comprising expression products according to this disclosure, about 0.01 μg/kg to about 100 mg/kg body weight will be administered, for example, by the intradermal, subcutaneous, intramuscular, or intravenous route, or by any route known in the art to be suitable under a given set of circumstances. A preferred dosage, for example, is about 1 μg/kg to about 20 mg/kg, with about 5 μg/kg to about 10 mg/kg particularly preferred. It will be evident to those skilled in the art that the number and frequency of administration will be dependent upon the response of the host.

The pharmaceutical compositions of this disclosure may be in any form that allows for administration to a patient, such as, for example, in the form of a solid, liquid, or gas (aerosol). The composition may be in the form of a liquid, e.g., an elixir, syrup, solution, emulsion or suspension, for administration by any route described herein.

A liquid pharmaceutical composition as used herein, whether in the form of a solution, suspension or other like form, may include one or more of the following components: sterile diluents such as water for injection, saline solution (e.g., physiological saline), Ringer's solution, isotonic sodium chloride, fixed oils such as synthetic mono- or digylcerides that may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; buffers such as acetates, citrates or phosphates; chelating agents such as ethylenediaminetetraacetic acid; and agents for the adjustment of tonicity such as sodium, chloride, or dextrose. The parenteral preparation can be enclosed in ampoules, disposable syringes or multiple dose vials made of glass or plastic. Physiological saline is a preferred additive. An injectable pharmaceutical composition is preferably sterile.

It may also be desirable to include other components in the preparation, such as delivery vehicles including aluminum salts, water-in-oil emulsions, biodegradable oil vehicles, oil-in-water emulsions, biodegradable microcapsules, and liposomes. Examples of adjuvants for use in such vehicles include N-acetylmuramyl-L-alanine-D-isoglutamine (MDP), lipopolysaccharides (LPS), glucan, IL-12, GM-CSF, γ-interferon, and IL-15.

While any suitable carrier known to those of ordinary skill in the art may be employed in the pharmaceutical compositions of this disclosure, the type of carrier will vary depending on the mode of administration and whether a sustained release is desired. For parenteral administration, the carrier may comprise water, saline, alcohol, a fat, a wax, a buffer, or any combination thereof. For oral administration, any of the above carriers or a solid carrier, such as mannitol, lactose, starch, magnesium stearate, sodium saccharine, talcum, cellulose, glucose, sucrose, magnesium carbonate, or any combination thereof, may be employed.

Also contemplated is the administration of multi-specific fusion protein compositions of this disclosure in combination with a second agent. A second agent may be one accepted in the art as a standard treatment for a particular disease state, such as inflammation, autoimmunity, and cancer. Exemplary second agents contemplated include cytokines, growth factors, steroids, NSAIDs, DMARDs, chemotherapeutics, radiotherapeutics, or other active and ancillary agents.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

This disclosure also contemplates a kit comprising a pharmaceutical composition in a unit dose or multi-dose container, e.g., a vial, and a set of instructions for administering the composition to patients suffering a disorder as described herein.

All U.S. patents, U.S. patent application publications, U.S. patent applications, foreign patents, foreign patent applications, non-patent publications, tables, sequences, webpages, or the like referred to in this specification, are incorporated herein by reference, in their entirety. The following examples are intended to illustrate, but not limit, this disclosure.

### EXAMPLES

### Xceptor Sequences

Exemplary IL6 antagonist variable region (V_{L} and V_{H}) binding sequences (SEQ ID NO:373-496) are disclosed herein. Also disclosed are amino acid sequences and nucleic acid expression cassettes for exemplary Xceptor fusion proteins comprising a TGFβR2 ectodomain and an anti-IL6xR binding domain. Xceptor fusion proteins having a TGFβR2 ectodomain at the amino-terminus and an anti-IL6xR binding domain at the carboxy terminus, are referred to herein as TRU(XB6)-1019.1 and TRU(XB6)-1019.2 (amino acid sequences provided in SEQ ID NO:737 and 738, respectively, with the corresponding nucleotide sequences being provided in SEQ ID NO:741 and 742, respectively). The Xceptor fusion proteins in the reverse orientation—that is, having an anti-IL6xR binding domain at the amino-terminus and a TGFβ2 ectodomain at the carboxy terminus, are referred to herein as TRU(X6B)-1019.1 and TRU(X6B)-1019.2 (amino acid sequences provided in SEQ ID NO:735 and 736, respectively, with the corresponding nucleotide sequences being provided in SEQ ID NO:739 and 740, respectively).

### Activity Examples

Various Xceptor fusion proteins described herein were tested for activity as described below. Abbreviations used in the following examples include the following terms, except where indicated otherwise:

PBS-T: PBS, pH 7.2-7.4 and 0.1% Tween®20

Working buffer: PBS-T with 1% BSA

Blocking buffer (PBS-T with 3% BSA

### Example 1

### TGFβ Binding Domains

A phage library of Fab binding domains is screened for binding domains specific for either a TGFβ or an IL6xR complex essentially as described by Hoet et al. (2005) Nature Biotechnol. 23:344. The binding domains are cloned by PCR amplification. Briefly, the VL and VH regions from the Fab library clones are amplified using PCR SuperMix (Invitrogen, San Diego, Calif.) and appropriate primers that create the G₄S linker via overlap, with an initial anneal at 56° C. for 9 cycles, then 62° C. for an additional 20 cycles. The PCR products are separated on an agarose gel and purified using a Qiagen (Chatsworth, Calif.) PCR Purification column. The second round sewing reaction involves mixing a molar equivalent of VL and VH products with Expand buffer and water, denaturing at 95° C. for 5 sec, then cooling slowly to room temperature. To amplify, a mix of dNTPs is added with Expand enzyme and incubated at 72° C. for 10 sec. The outside primers are added (5′ VH and 3′ VL) and the mix is cycled 35 times with an anneal at 62° C. and a 45 min extension reaction. The resulting 750 basepair product is gel purified, digested with EcoRI and NotI, and cloned in plasmid pD28 (for more details, see US Patent Application Publication No. 2005/0136049 and PCT Application Publication No. WO 2007/146968).

### Example 2

### Xceptor Binding to IL6 and Hyper IL6 by ELISA

Hyper-IL6 (HIL6 or IL6xR), recombinant human IL6 (rhIL6), and human soluble IL6R binding activity was examined for exemplary Xceptors TRU(XT6)-1002, 1019, 1025, 1042, 1058, and TRU(X6T)-1019 (SEQ ID NO:608, 625, 631, 648, 664 and 670, respectively), substantially as follows. Each of these Xceptors includes a TNFRSF1B ectodomain and an anti-IL6xR binding domain.

### HIL6 and IL6 Binding

Added to each well of a 96-well plate was 100 μl goat anti-human IgG-Fc (Jackson ImmunoResearch, West Grove, Pa.) from a 2 μg/ml solution in PBS, pH 7.2-7.4. The plate was covered, and incubated overnight at 4° C. After washing four times with PBS (pH 7.2-7.4) and 0.1% Tween®20 (PBS-T), 250 μl Blocking buffer (PBS-T with 3% BSA or 10% normal goat serum) was added to each well, the plate was covered, and incubated at room temperature for 2 hours (or at 4° C. overnight). After washing the plate three times with PBS-T, added in duplicate wells to the anti-human IgG-Fc coated plate was 100 μl/well Xceptor TNFRSF1B::anti-HIL6 samples and human gp130-Fc chimera (R&D Systems, Minneapolis, Minn.) serially diluted three-fold in Working buffer starting at 300 ng/ml, the plate was covered, and incubated at room temperature for about 1 to 2 hours. After washing the plate five times with PBS-T, added in duplicate wells was 100 μl/well human Hyper IL-6 or recombinant human IL-6 from a 150 pM solution in Working buffer, the plate was covered, and incubated at room temperature for about 1 to 2 hours. After washing the plate five times with PBS-T, 100 μl/well anti-human IL-6-biotin (R&D Systems) from a 150 ng/ml solution in Working buffer, the plate was covered, and incubated at room temperature for about 1 to 2 hours. After washing the plate five times with PBS-T, 100 μl per well horse radish peroxidase-conjugated streptavidin (Zymed, San Francisco, Calif.) diluted 1:4,000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for about 3 to 5 minutes and then the reaction was stopped with 50 μl Stop buffer (1N H₂SO₄) per well. The absorbance of each well was read at 450 nm.

sIL6R Binding

Added to each well of a 96-well plate was 100 μl goat anti-human IgG-Fc (ICN Pharmaceuticals, Costa Mesa, Calif.) from a 2 μg/ml solution in PBS, pH 7.2-7.4, The plates were covered, and incubated overnight at 4° C. After washing four times with PBS-T, 250 μl Blocking buffer (PBS-T with 3% BSA or 10% normal goat serum) was added to each well, the plate was covered, and incubated at room temperature for 2 hours (or at 4° C. overnight). After washing the plate three times with PBS-T, added in duplicate wells to the anti-human IgG-Fc coated plate was 100 μl/well Xceptor TNFRSF1B::anti-HIL6 samples, positive control anti-human IL-6R(R&D Systems, Minneapolis, Minn.) and negative controls human IgG or human gp130-Fc chimera (R&D Systems), each serially diluted three-fold in Working buffer starting at 300 ng/ml, the plate was covered, and incubated at room temperature for about 1 to 2 hours. After washing the plate five times with PBS-T, added in duplicate wells was 100 μl/well recombinant human sIL-6R(R&D Systems) from a 75 pM solution in Working buffer, the plate was covered, and incubated at room temperature for about 1 to 2 hours. After washing the plate five times with PBS-T, added 100 μl/well anti-human IL-6R-biotin (R&D Systems) from a 100 ng/ml solution in Working buffer, the plate was covered, and incubated at room temperature for about 1 to 2 hours. After washing the plate five times with PBS-T, 100 μl per well horse radish peroxidase-conjugated streptavidin (Zymed, San Francisco, Calif.) diluted 1:4,000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for about 3 to 5 minutes and then the reaction was stopped with 50 μl Stop buffer (1N H₂SO₄) per well. The absorbance of each well was read at 450 nm.

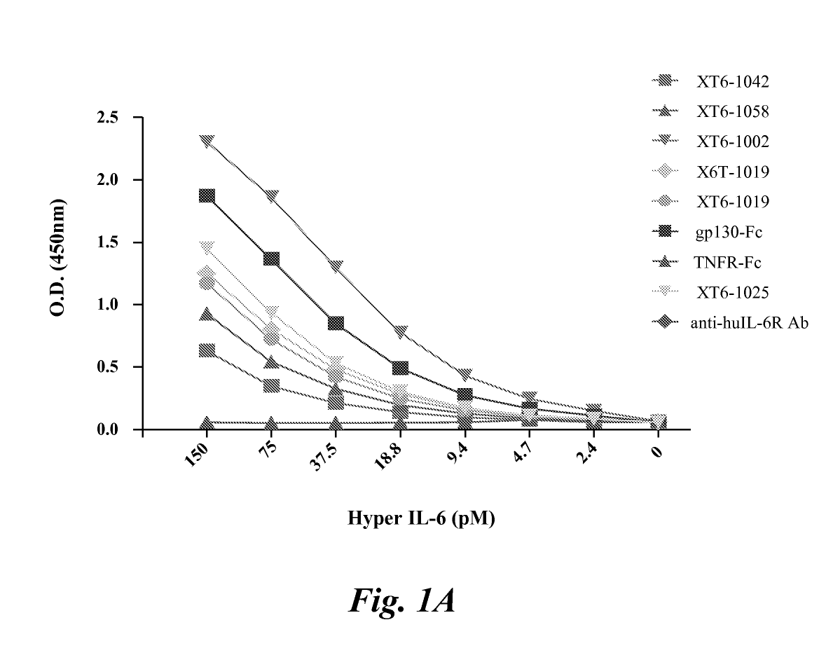

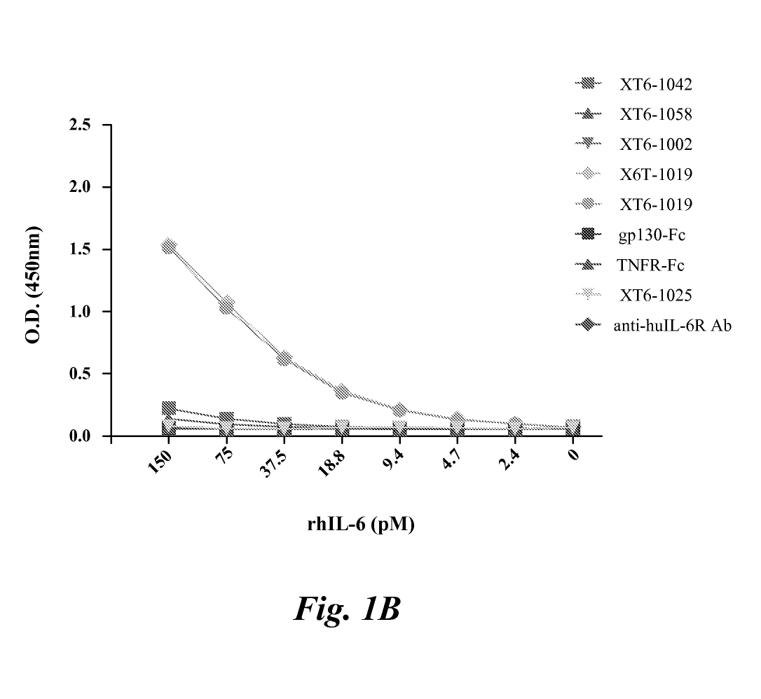

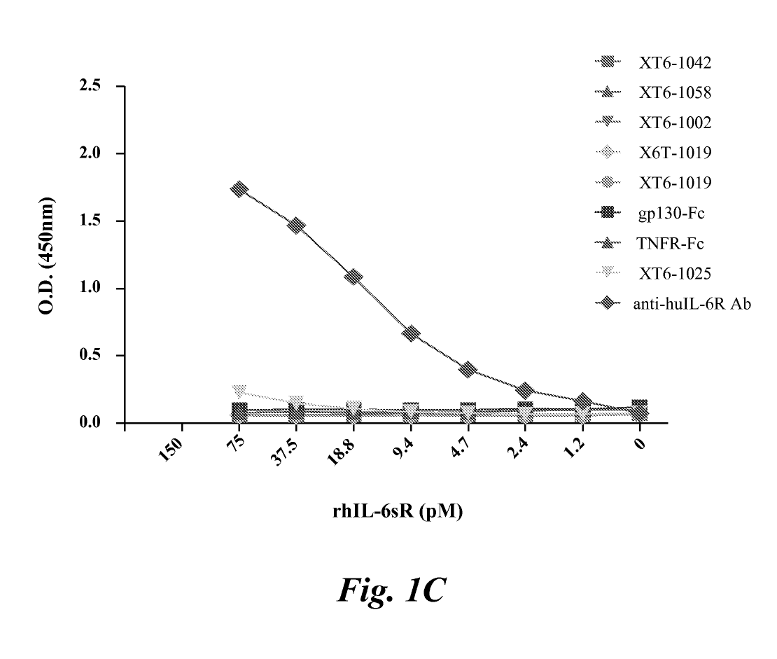

The data in FIGS. 1A-1C demonstrate that all Xceptor fusion proteins, whether the TNFRSF1B ectodomain was on the amino- or carboxy terminus of the fusion protein molecules, can bind HIL6. Furthermore, these assays show that the Xceptor proteins have specificity for the IL6xR complex because only two of the Xceptors bind rhIL6 (FIG. 1B) and none bind sIL6R (FIG. 1C). In related studies, the xceptor TRU(XT6)-1002 and the SMIP TRU(S6)-1002 were found to cross-react with IL6 from the non-human primate Mucaca mulatta.

### Example 3

### Xceptor Binding to TNF-α by ELISA

TNF-α binding activity was examined for Xceptors TRU(XT6)-1002, 1042, 1058, 1019, and TRU(X6T)-1019 (SEQ ID NO:608, 648, 664, 625 and 670, respectively), substantially as follows.

Added to each well of a 96-well plate was 100 μl goat anti-human IgG-Fc (ICN Pharmaceuticals, Costa Mesa, Calif.) from a 2 μg/ml solution in PBS, pH 7.2-7.4. The plate was covered, and incubated overnight at 4° C. After washing four times with PBS-T, 250 μl Blocking buffer was added to each well, the plate was covered, and incubated at room temperature for 2 hours (or at 4° C. overnight). After washing the plate three times with PBS-T, added in duplicate wells to the anti-human IgG-Fc coated plate was 100 μl/well Xceptor TNFRSF1B::anti-HIL6 samples, positive controls Enbrel® (etanercept) and recombinant human TNFR2 (TNFRSF1B)-Fc chimera (R&D Systems, Minneapolis, Minn.), and negative controls human IgG or human gp130-Fc chimera (R&D Systems), each serially diluted three-fold in Working buffer starting at 300 ng/ml, the plate was covered, and incubated at room temperature for about 1 to 2 hours. After washing the plate five times with PBS-T, added in duplicate wells was 100 μl/well recombinant human TNF-α (R&D Systems) from a 2 ng/ml solution in Working buffer, the plate was covered, and incubated at room temperature for about 1 to 2 hours. After washing the plate five times with PBS-T, added 100 μl/well anti-human TNF-α-biotin (R&D Systems) from a 200 ng/ml solution in Working buffer, the plate was covered, and incubated at room temperature for about 1 to 2 hours. After washing the plate five times with PBS-T, 100 μl per well horse radish peroxidase-conjugated streptavidin (Jackson ImmunoResearch, West Grove, Pa.) diluted 1:1,000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for about 3 to 5 minutes and then the reaction was stopped with 50 μl Stop buffer (1N H₂SO₄) per well. The absorbance of each well was read at 450 nm.

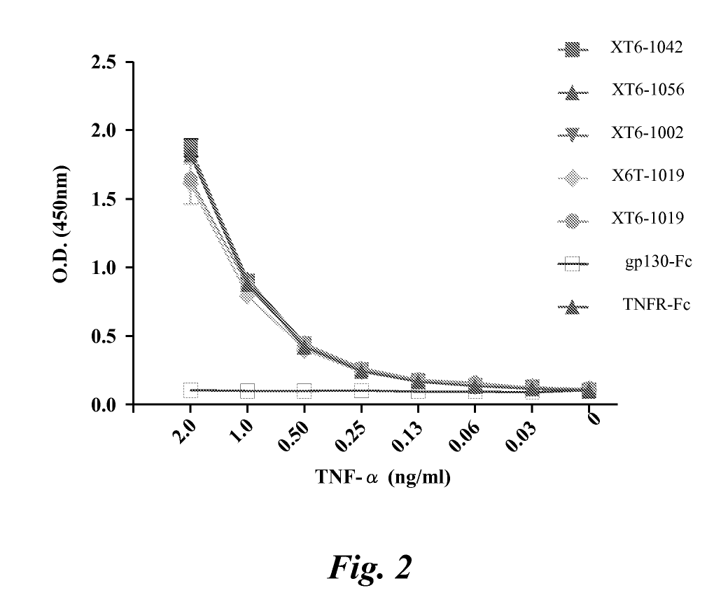

The data in FIG. 2 shows that all Xceptor fusion proteins tested can bind TNF-α, whether the TNFRSF1B ectodomain was on the amino- or carboxy terminus of the fusion protein.

### Example 4

### Xceptor Dual Ligand Binding by ELISA

Concurrent binding to TNF-α and to IL6xR complex was examined for Xceptor fusion protein TRU(XT6)-1006 (SEQ ID NO:612), substantially as follows.

Added to each well of a 96-well plate was 100 μl human HIL-6 solution (5 μg/ml in PBS, pH 7.2-7.4). The plate was covered, and incubated overnight at 4° C. After washing four times with PBS-T, then 250 μl Blocking buffer was added to each well, the plate was covered, and incubated at room temperature for 2 hours (or at 4° C. overnight). After washing the plate three times with PBS-T, added in duplicate wells to the HIL-6 coated plate was 100 μl/well Xceptor TNFRSF1B::HIL6 samples serially diluted three-fold in Working buffer starting at 300 ng/ml. Negative controls included human gp130-Fc chimera (R&D Systems, Minneapolis, Minn.), Enbrel® (etanercept), and Working buffer only. The plate was covered and incubated at room temperature for 1.5 hours. After washing the plate five times with PBS-T, 100 μl per well recombinant human TNF-α (R&D Systems, Minneapolis, Minn.) to 2 ng/ml in Working buffer was added, the plate was covered, and incubated at room temperature for 1.5 hr. After washing the plate five times with PBS-T, 100 μl per well anti-human TNF-α-biotin (R&D Systems) to 200 ng/ml in Working buffer was added, the plate was covered, and incubated at room temperature for 1.5 hr. After washing the plate five times with PBS-T, 100 μl per well horse radish peroxidase-conjugated streptavidin (Jackson ImmunoResearch, West Grove, Pa.) diluted 1:1000 in Working buffer was added, the plate was covered, and incubated at room temperature for 30 minutes. After washing the plate six times with PBS-T, 100 μl per well 3,3,5,5-tetramentylbenzidine (TMB) substrate solution (Pierce, Rockford, Ill.) was added for 3-5 minutes and then the reaction was stopped with 50 μl Stop buffer (1N H₂SO₄) per well. The absorbance of each well was read at 450 nm.

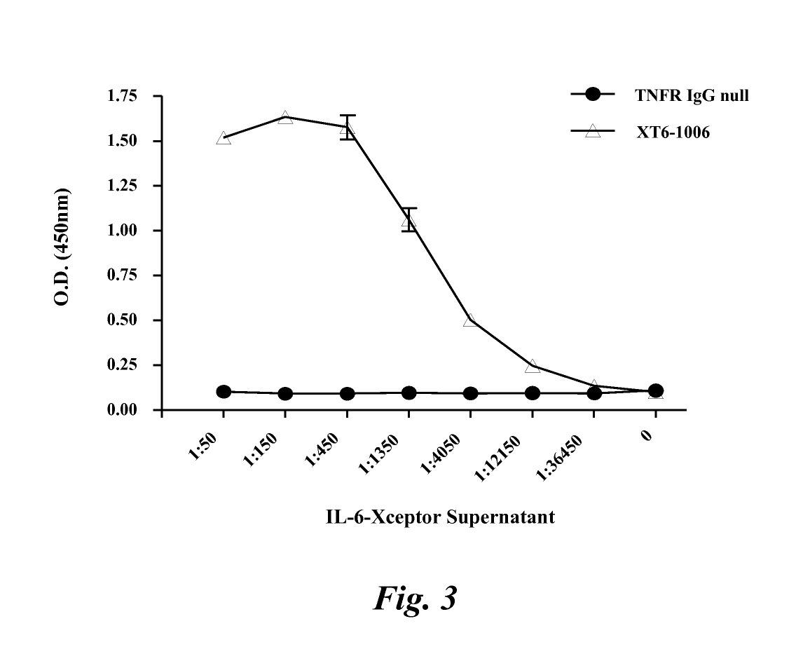

The data in FIG. 3 demonstrates that Xceptor proteins can bind two ligands simultaneously (in this case TNF-α and Hyper IL6).

### Example 5

### Xceptor Blocking of Hyper IL6 Binding to gp130 by ELISA

Blocking of Hyper IL6 (IL6xR) binding to soluble gp130 receptor by Xceptor fusion proteins TRU(XT6)-1004, 1006, 1007, 1008, 1013, and 1019 (SEQ ID NO:610, 612, 613, 614, 619 and 625, respectively), was examined substantially as follows.

Added to each well of a 96-well plate was 100 μl human gp130-Fc chimera (R&D Systems, Minneapolis, Minn.) from of 0.25-0.5 μg/ml solution in PBS, pH 7.2-7.4. The plates were covered, and incubated overnight at 4° C. After washing four times with PBS-T, 250 μl Blocking buffer (PBS-T with 3% BSA or 10% normal goat serum) was added to each well, the plate was covered, and incubated at room temperature for 2 hours (or at 4° C. overnight). Serial five-fold dilutions in Working buffer starting at 50 μg/ml were made of the following samples: Xceptor TNFRSF1B::anti-HIL6 samples, positive controls human gp130-Fc chimera (R&D Systems) and anti-human IL-6R(R&D Systems), and negative controls anti-human IL-6 (R&D Systems), human IgG or Enbrel® (etanercept). Equal volumes of the serially diluted Xceptor samples were mixed with Hyper IL-6 (final Hyper IL-6 concentration of 2.5 ng/ml) and incubated at room temperature for 1 hour. After washing the plate three times with PBS-T, added in duplicate wells to the human gp130-Fc coated plate was 100 μl/well of the serially dilutions of Xceptor/HIL6 mixtures, human gp130-Fc chimera, anti-human IL-6R, anti-human IL-6, human IgG, and Enbrel® (etanercept), the plate was covered, and incubated at room temperature for about 1.5 hours. After washing the plate five times with PBS-T, 100 μl per well horse radish peroxidase-conjugated anti-mouse IgG-Fc (Pierce, Rockford, Ill.) diluted 1:10,000 in Working buffer was added, the plate was covered, and incubated at room temperature for 1 hour. After washing the plate six times with PBS-T, 100 μl per well 3,3,5,5-tetramentylbenzidine (TMB) substrate solution (Pierce) was added for about 5 to 15 minutes and then the reaction was stopped with 50 μl Stop buffer (1N H₂SO₄) per well. The absorbance of each well was read at 450 nm.

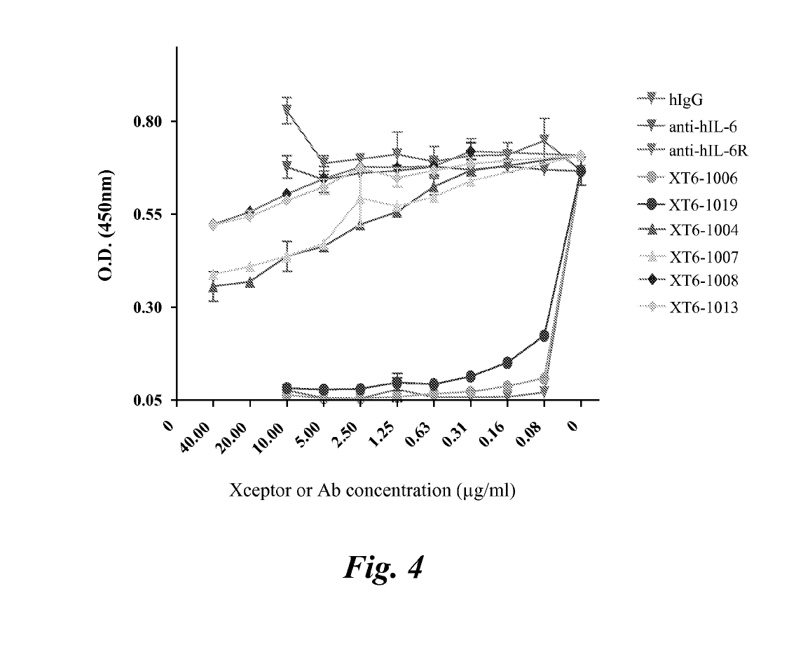

The data in FIG. 4 demonstrate that Xceptor proteins comprising an anti-IL6xR binding domain can block soluble gp130 from binding to HIL6.

### Example 6

### Xceptor Blocking of IL6 and Hyper IL6 Induced Cell Proliferation

Blocking of IL6 or Hyper IL6 (IL6xR) induced cell proliferation of TF-1 cells was examined for Xceptor fusion proteins TRU(XT6)-1011, 1014, 1025, 1026, 1002, and TRU(X6T)-1019 (SEQ ID NO:617, 620, 631, 632, 608 and 670, respectively), substantially as follows.

Added to each well of a 96-well flat bottom plate were 0.3×10⁶ TF-1 cells (human erythroleukemia cells) in the fresh growth medium (10% FBS-RPMI 1640; 2 mM L-glutamine; 100 units/ml penicillin; 100 μg/ml streptomycin; 10 mM HEPES; 1 mM sodium pyruvate; and 2 ng/ml Hu GM-CSF) one day before use in proliferation assay. The cells were then harvested and washed twice with assay medium (same as growth medium except without GM-CSF, cytokine-free), then resuspended at 1×10⁵ cells/ml in assay medium. For blocking IL-6 activity, serial dilutions of a TNFSFR1B::anti-HIL-6 Xceptor of interest or antibody was pre-incubated with a fixed concentration of recombinant human IL-6 (rhIL-6) (R&D Systems, Minneapolis, Minn.) or hyper IL-6 (HIL-6) in 96-well plates for 1 hour at 37° C., 5% CO₂. Controls used included human IgG; human gp130-Fc chimera (R&D Systems); anti-hIL-6 antibody (R&D Systems); and anti-hIL-6R antibody (R&D Systems). After the pre-incubation period, 1×10⁴1 cells (in 100 μl) was added to each well. The final assay mixture, in a total volume of 200 μL/well, containing TNFSFR1B::HIL-6, rhIL-6, or HIL-6 and cells was incubated at 37° C., 5% CO₂ for 72 hours. During the last 4-6 hours of culture, ³H-thymidine (20 μCi/ml in assay medium, 25 μL/well) was added. The cells were harvested onto UniFilter-96 GF/c plates and incorporated ³H-Thymidine was determined using TopCount reader (Packard). The data are presented as the Mean of cpm±SD of triplicates. The percentage of blocking=100−(test cpm−control cpm/maximum cpm−control cpm)*100.

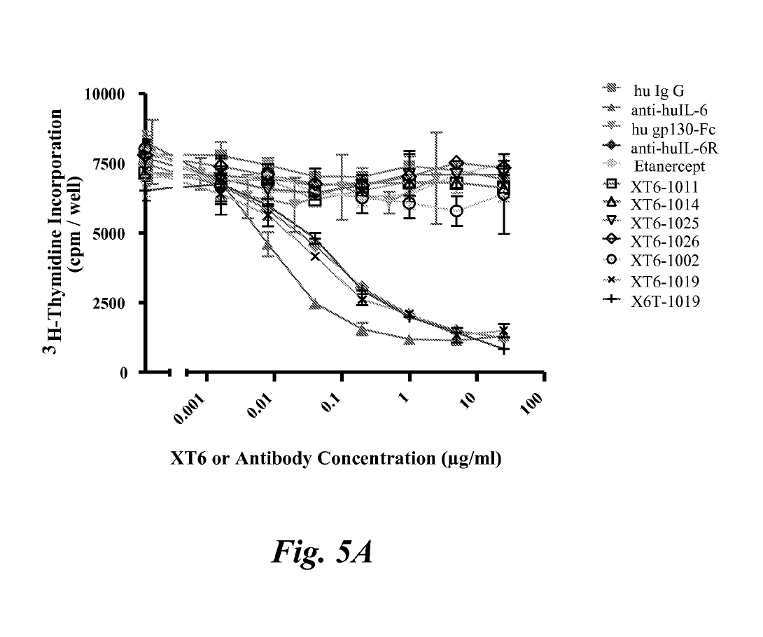

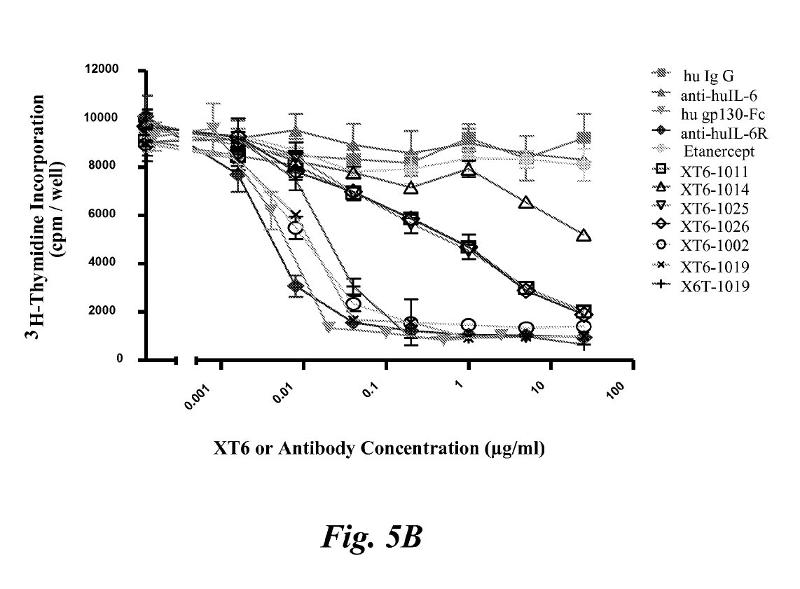

The data in FIG. 5A and FIG. 5B demonstrate that all Xceptor proteins, whether the TNFRSF1B ectodomain was on the amino- or carboxy terminus of the fusion protein molecules, can block cell proliferation induced by IL6 or Hyper IL6, respectively, or both.

### Example 7

### Xceptor Blocking of TNF-α Binding to TNFR by ELISA

Blocking of TNF-α binding to TNF receptor by Xceptor fusion proteins TRU(XT6)-1004, 1006, 1007, 1008, 1013, and 1019 (SEQ ID NO:610, 612, 613, 614, 619 and 625, respectively) was examined substantially as follows.

Added to each well of a 96-well plate was 100 μL recombinant human TNFR2-Fc chimera (R&D Systems, Minneapolis, Minn.) from of 0.25-0.5 μg/ml solution in PBS, pH 7.2-7.4. The plates were covered, and incubated overnight at 4° C. After washing four times with PBS-T, 250 μL Blocking buffer (PBS-T with 3% BSA or 10% normal goat serum) was added to each well, the plate was covered, and incubated at room temperature for 2 hours (or at 4° C. overnight). Serial five-fold dilutions in Working buffer starting at 50 to 250 μM were made of the following samples: Xceptor TNFRSF1B::anti-HIL6 samples, positive controls Enbrel® (etanercept) and anti-TNF-α (R&D Systems), and negative controls human gp130-Fc chimera (R&D Systems) and human IgG. Equal volumes of the serially diluted Xceptor samples were mixed with TNF-α (final TNF-α concentration of 2.5 ng/ml) and incubated at room temperature for 1 hour. After washing the plate three times with PBS-T, added in duplicate wells to the recombinant human TNFR2-Fc coated plate was 100 μl/well of the serially dilutions of Xceptor/TNF-α mixture, Enbrel® (etanercept), anti-TNF-α, human gp130-Fc chimera, and human IgG, the plate was covered, and incubated at room temperature for about 1.5 hours. After washing the plate five times with PBS-T, 100 μL per well of anti-human TNF-α-biotin (R&D Systems) from a 200 ng/ml solution in Working buffer was added, the plate was covered, and incubated at room temperature for 1 to 2 hours. After washing the plate five times with PBS-T, 100 μL per well horse radish peroxidase-conjugated streptavidin (Jackson ImmunoResearch, West Grove, Pa.) diluted 1:1,000 in Working buffer was added, the plate was covered, and incubated at room temperature for 30 minutes. After washing the plate six times with PBS-T, 100 μL per well 3,3,5,5-tetramentylbenzidine (TMB) substrate solution (Pierce, Rockford, Ill.) was added for about 3 to 5 minutes and then the reaction was stopped with 50 μl Stop buffer (1N H₂SO₄) per well. The absorbance of each well was read at 450 nm.

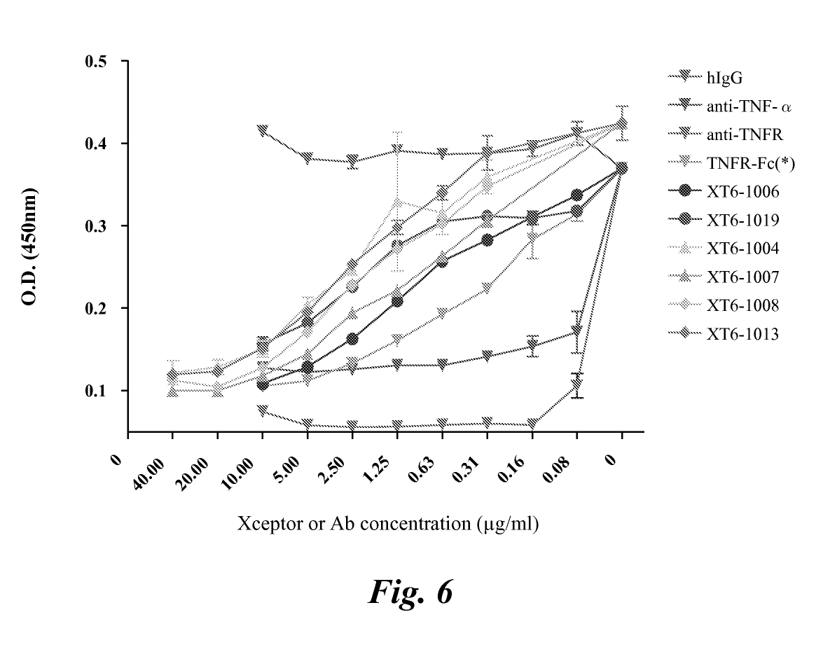

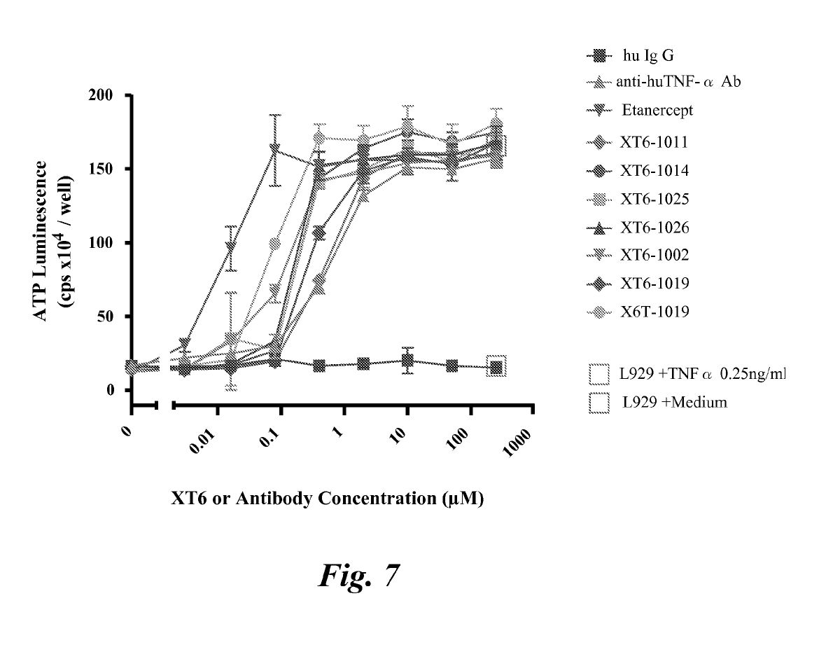

The data in FIG. 6 show that Xceptor proteins blocked TNF-α binding to TNF receptor, which was approximately equivalent to blocking by TNFR-Fc.

### Example 8

### Xceptor Blocking of TNF-α Induced Cell Killing

Blocking of TNF-α induced killing of L929 cells was examined for Xceptor fusion proteins TRU(XT6)-1011, 1014, 1025, 1026, 1002, and TRU(X6T)-1019 (SEQ ID NO:617, 620, 631, 632, 608 and 670, respectively), substantially as follows.

A suspension of L929 mouse fibroblast cells (ATCC, Manassas, Va.) was prepared at a density of 2×10⁵ cells/ml in culture medium (10% FBS-RPMI 1640; 2 mM L-glutamine; 100 units/ml penicillin; 100 μg/ml streptomycin; and 10 mM HEPES), then 100 μl was added to each well of a 96-well flat bottom black plate and incubated overnight at 37° C., 5% CO₂ in a humidified incubator. Xceptor TNFRSF1B::anti-HIL6 samples serially diluted in assay medium (same as culture medium but supplemented with 2% FBS) were mixed with an equal volume of recombinant human TNF-α (rhTNF-α; R&D Systems, Minneapolis, Minn.), and incubated at 37° C., 5% CO₂ in a humidified incubator for 1 hour. Positive controls (i.e., those agents that block TNF-α induced killing of L929 cells) included Enbrel® (etanercept), rhTNFR2-Fc chimera (R&D Systems, Minneapolis, Minn.), and anti-TNF-α antibody (R&D Systems, Minneapolis, Minn.). Negative controls included assay medium alone (no TNF-α added) and antibody hIgG (with TNF-α added). To analyze TNF-α activity, culture medium was removed from the L929 cells and then each well received 50 μl of a TNF-α/Xceptor or control mixture, and 50 μl actinomycin D (Sigma-Aldrich, St. Louis, Mo.) (from a freshly prepared working solution of 4 μg/ml). The cells were then incubated for 24 hrs at 37° C., 5% CO₂ in a humidified incubator. To measure cell viability, added to each well was 100 μl ATPlite 1 Step Reagent (PerkinElmer, Waltham, Mass.) according to the manufacturer's instructions, shaken for two minutes, and then luminescence is measured using a TopCount reader (Packard).

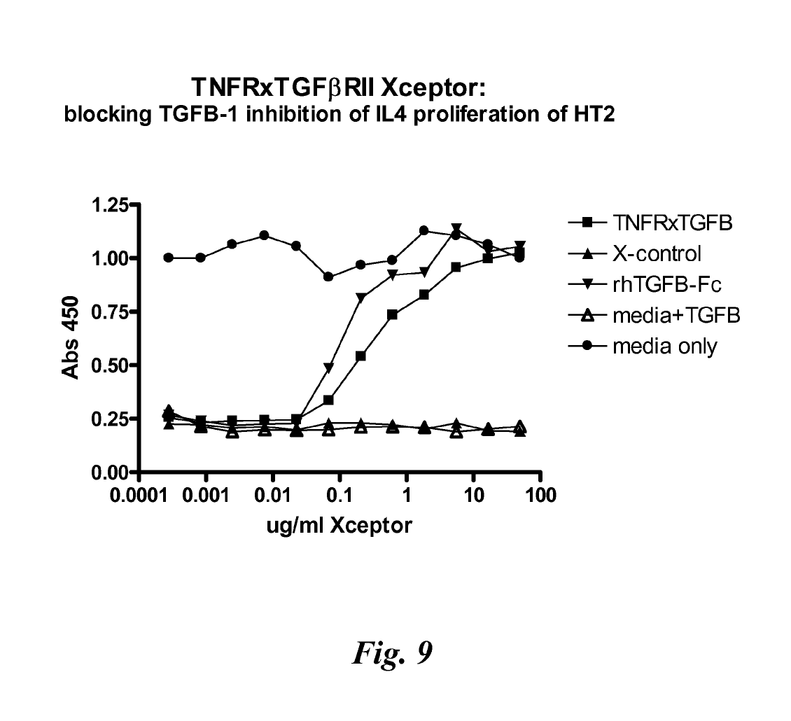

The data in FIG. 9 demonstrate that all Xceptor proteins, whether the TNFRSF1B ectodomain was on the amino- or carboxy terminus of the fusion protein molecules, can block TNF-α induced cell killing in this assay.

### Example 9

### Xceptor Binding to TGFβ by ELISA

TGFβ binding activity was examined for Xceptors X6B and XB6, substantially as follows.

Added to each well of a 96-well plate was 100 μl goat anti-human IgG-Fc (ICN Pharmaceuticals, Costa Mesa, Calif.) from a 2 μg/ml solution in PBS, pH 7.2-7.4. The plate was covered, and incubated overnight at 4° C. After washing four times with PBS-T, 250 μl Blocking buffer (PBS-T with 10% NGS) was added to each well, the plate was covered, and incubated at room temperature for 2 hours. After washing the plate three times with PBS-T, added in duplicate wells to the anti-human IgG-Fc coated plate was 100 μl/well Xceptor TGFβR2::anti-HIL6 samples, positive control recombinant human TGFβRII-Fc chimera (R&D Systems, Minneapolis, Minn.), and negative control recombinant human TNFR2 (TNFRSF1B)-Fc chimera (R&D Systems), each diluted to 300 ng/ml in Working buffer (PBS-T with 1% BSA). The plate was covered, and incubated at room temperature for about 1 hour. After washing the plate five times with Wash buffer (PBS/0.1% Tween 20 (PBS-T)), added was 100 μl/well TGFβ-1 ligand (R&D Systems), serially diluted two-fold in Working buffer starting at 4 ng/ml. The plate was covered, and incubated at room temperature for 1 hour. After washing the plate five times with Wash buffer, added was 100 μl/well biotinylated anti-TGFβ-1 (R&D Systems) from a 200 ng/ml solution in Working buffer. The plate was covered and incubated at room temperature for 1 hour. After washing the plate five times with Wash buffer, 100 μl per well horse radish peroxidase-conjugated streptavidin (Pierce Rockford, Ill.) diluted 1:20,000 in Working buffer was added, the plate was covered, and incubated at room temperature for 30 minutes. After washing the plate five times with Wash buffer, 100 μl per well QuantaBlu Fluorogenic Peroxidase Substrate solution (Pierce, Rockford, Ill.; prepared by mixing 9 ml substrate solution with 1 ml peroxide solution) was added, and the plate was covered and incubated at room temperature for 20 min. The reaction was stopped with 50 μl QuantaBlu Stop Solution per well. The absorbance of each well was read at 325 nm.

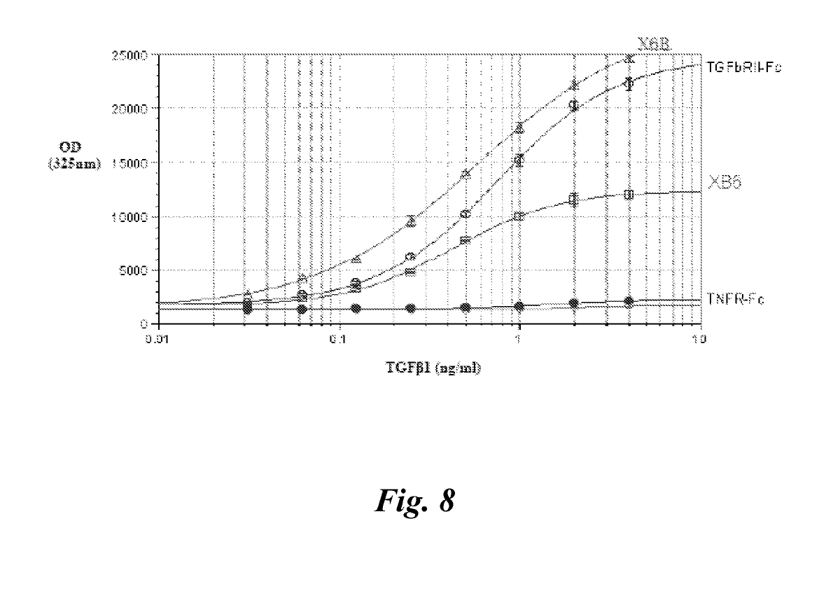

The data in FIG. 8 shows that the Xceptor fusion proteins tested bound TGFβ-1, whether the TGFβ2 ectodomain was on the amino- or carboxy terminus of the fusion protein.

### Example 10

### Expression of Xceptor Fusion Proteins

Expression of certain of the Xceptor fusion proteins disclosed herein in 293 cells was performed using the FreeStyle™ 293 Expression System (Invitrogen, Carlsbad, Calif.) according to the manufacturer's instructions.

For each 30 ml transfection, 3×10⁷ cells in 28 ml of FreeStyle™ 293 Expression Medium were used. On the day of transfection, a small aliquot of the cell suspension was transferred to a microcentrifuge tube, and the viability and the amount of cell clumping determined using the trypan blue dye exclusion method. The suspension was vigorously vortexed for 45 seconds to break up cell clumps and total cell counts determined using a Coulter Counter or a hemacytometer. The viability of the cells was over 90%. A shaker flask containing the required cells was placed in a 37° C. incubator on an orbital shaker.

For each transfection sample, lipid-DNA complexes were prepared as follows. 30 μg of plasmid DNA was diluted in Opti-MEM® I to a total volume of 1 ml and mixed gently. 60 μl of 293Fectin™ was diluted in Opti-MEM® I to a total volume of 1 ml, mixed gently, and incubated for 5 minutes at room temperature. After the 5 minute incubation, the diluted DNA was added to the diluted 293Fectin™ to obtain a total volume of 2 ml and mixed gently. The resulting solution was incubated for 20-30 minutes at room temperature to allow DNA-293Fectin™ complexes to form.

While the DNA-293Fectin™ complexes were incubating, the cell suspension was removed from the incubator and the appropriate volume of cell suspension was placed in a sterile, disposable 125 ml Erlenmeyer shaker flasks. Fresh, pre-warmed FreeStyle™ 293 Expression Medium was added up to a total volume of 28 ml for a 30 ml transfection.

After the DNA-293Fectin™ complex incubation was complete, 2 ml of DNA-293Fectin™ complex was added to the shaker flasks. 2 ml of Opti-MEM® I was added to the negative control flask, instead of DNA-293Fectin™ complex. Each flask contained a total volume of 30 ml, with a final cell density of approximately 1×10⁶ viable cells/ml. The cells were incubated in a 37° C. incubator with a humidified atmosphere of 8% CO₂ in air on an orbital shaker rotating at 125 rpm. Cells were harvested at approximately 7 days post-transfection and assayed for recombinant protein expression.

Xceptor molecules having a TNFRSF1B ectodomain and a TGFβRII ectodomain were expressed in 293 cells as described above.

### Example 11

### Xceptor Binding to Ligands by ELISA

The ability of xceptor molecules comprising a TNFRSF1B ectodomain and either a TWEAKR ectodomain, an OPG ectodomain, a TGFβRII ectodomain or an IL7R ectodomain to bind to the ligands TWEAK, RANKL, TGFβ or IL7, respectively, was examined substantially as follows.

Mouse and human ligands (R&D Systems, Minnesota, Minn.) were added to wells of a 96-well plate at a concentration of 1 μg/ml in PBS (100 μL/well). Plates were incubated at 4° C. overnight. After washing five times with PBS-T, 250 μL Blocking Buffer (PBS-T with 3% BSA) was added to each well, and the plate covered and incubated at room temperature (RT) for 2 hours. Serial three fold dilutions of xceptors were made in Working Buffer (PBS-T with 1% BSA) starting at 300 ng/ml. As a negative control, an irrelevant xceptor was used. The plate was incubated at RT for 1 hour. After washing five times with PBS-T, 100 μL per well of HRP-conjugated anti-human IgG-Fc (1:5000 in Working buffer) was added, the plate covered, and incubated at RT for 1 hour. After washing five times with PBS-T, 100 μL of Quant-Blu substrate (Pierce, Rockford, Ill.) was added to each well. The plate was incubated at RT for 10-30 minutes, and fluorescence measured at 325/420 nm.

The results are shown in Table 3 below. The binding of the TNFRxTGFβRII to mouse TGFβ was not tested, however it is noted that mouse and human TGFβ are 99% identical.

TABLE 3
Xceptor binding to Ligands

| | | Mouse ligand | Human ligand |
|---|---|---|---|
| TNFR x R | Ligand | binding | binding |
| TNFR x TWEAKR | TWEAK | +++ | +++ |
| TNFR x OPG | RANKL | +++ | +++ |
| TNFR x TGFβRII | TGFβ | homologous | +++ |
| TNFR x IL7R | IL7 | ND | + |

ND = Not Done

### Example 12

### Xceptor Blocking of TGFβ-1-Induced Inhibition of Cell Proliferation

Blocking of TGFβ-1 induced inhibition of IL-4 proliferation of HT2 cells was examined for the Xceptor of SEQ ID NO:1236 using the method described by Tsang et al. (Tsang, M. et al. (1995) Cytokine 7:389).

Briefly, in a 96 well plate, Xceptor TNFR::TGFβRII samples were serially diluted in culture medium (RPMI, 10% FCS, 0.05 mM beta-mercaptoethanol) containing 1 ng/ml of human TGFβ-1; 100 ul per well. The plate was incubated at 37° C., 5% CO₂ in a humidified incubator for 1.5 hours. Negative controls included an irrelevant xceptor protein (with TGFβ-1 added) and culture medium (with and without TGFβ-1 added). The positive control was a recombinant TGFβRII-Fc chimera (R&D Systems, Minneapolis, Minn.). Following incubation, 1×10⁴ HT2 cells (ATCC, Manassas, Va.) in 100 ul of culture medium containing 15 ng/ml mIL4 (R&D Systems, Minneapolis, Minn.) was added to each well. The plate was then incubated at 37° C., 5% CO₂ in a humidified incubator for 72 hours.

To analyze TGFβ-1 activity by measuring cell viability, 100 ul of culture medium was removed from each well and replaced with 10 μL WST-8 reagent (Dojindo Molecular Technologies, Rockville, Md.). The plate was incubated for 2 hours at 37° C., and absorbance for each well read at 450 nM.

The data in FIG. 9 shows that the xceptor protein blocked TGFβ-1 inhibition of IL4-mediated proliferation of HT2 cells.

### Example 13

### Specificity of Binding to Hyper IL6 and not Other gp130 Cytokines

The effect of Xceptor fusion proteins on induction of TF-1 cell proliferation by IL6 and the gp130 cytokines IL-11, leukemia inhibitory factor (LIF), oncostatin M (OSM) and cardiotrophin-1 (CT-1) was examined substantially as follows.

Added to each well of a 96-well flat bottom plate was 0.3×10⁶ TF-1 cells (human erythroleukemia cells) in fresh growth medium (10% FBS-RPMI 1640, 2 mM L-glutamine, 100 units/ml penicillin, 100 μg/ml streptomycin, 10 mM HEPES, 1 mM sodium pyruvate and 2 ng/ml Hu GM-CSF) one day before use in the proliferation assay. The cells were harvested and washed twice with assay medium (same as growth medium except without GM-CSF, cytokine-free), then resuspended at 1×10⁵ cells/ml in assay medium. For examining blocking of LIF, OSM, and CT-1 activity, serial dilutions of a TNFSFR1B::anti-HIL-6 xceptors TRU(XT6)-1002 (SEQ ID NO:608), TRU(XT6)-1019 (SEQ ID NO:625), TRU(XT6)-1022 (SEQ ID NO:628), and TRU(XT6)-1025 (SEQ ID NO:631) were pre-incubated with a fixed concentration of each gp130 cytokine individually or hyper IL-6 (HIL-6) in 96-well plates for 1 hour at 37° C., 5% CO₂. After the pre-incubation period, 1×10⁴ cells (in 100 μl) were added to each well. The final assay mixture, in a total volume of 200 μL/well, containing TNFSFR1B::HIL-6, gp130 cytokine or HIL-6 and cells, was incubated at 37° C., 5% CO₂ for 72 hours. During the last 4-6 hours of culture, ³H-thymidine (20 μCi/ml in assay medium, 25 μL/well) was added. The cells were harvested onto UniFilter-96 GF/c plates and incorporated ³H-Thymidine was determined using TopCount reader (Packard). The percentage of blocking=100−(test cpm−control cpm/maximum cpm−control cpm)*100.

The results showed that the xceptor blocked IL6 activity but not IL-11, LIF, OSM or CT-1 (data not shown), and therefore bound to hyper IL6 but had no effect on the other gp130 cytokines tested.

### Example 14

### SMIP and Xceptor Binding to IL6R on Liver Cells

The ability of TRU(S6)-1002, TRU(XT6)-1019 and the anti-IL6 antibody hu-PM1 to bind to IL6R on the liver-derived HepG2 cells was examined as follows.

HepG2 cells were washed in FACS Buffer and adjusted to 2×10⁶ cells/mL in FACS Buffer (PBS+3% FBS). To wells of a 96-well plate were added 50 μL of this solution (10⁵ cells/well). The plates were held at 37° C. until ready to add diluted test molecules. Serial dilutions of the test molecules were prepared in FACS Buffer to give a 2× working stock which was diluted to 1× when added to cells. The diluted test molecules were added to cells (50 μL/well) and the cells incubated for 20 min on ice. Whole IgG was used as a control. The cells were then washed two times with FACS Buffer and resuspended in phycoerythrin-conjugated goat anti-human antibody (Jackson Labs; diluted 1:200 in FACS Buffer). After being incubated for 20 min on ice in the dark, the cells were washed two times with FACS buffer, resuspended in 200 ul PBS and read on a LSRII™ flow cytometer (BD Biosciences, San Jose, Calif.).

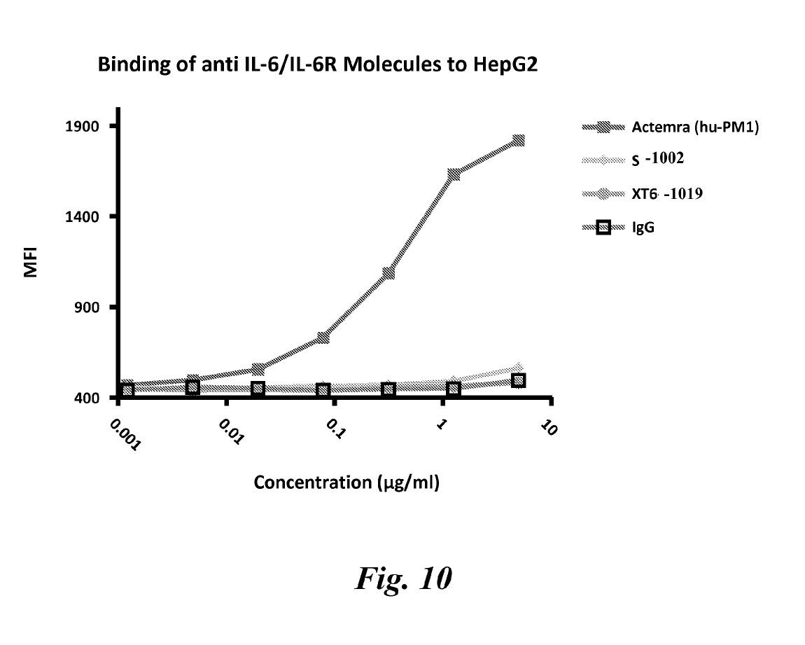

As shown in FIG. 10, TRU(S6)-1002 and TRU(XT6)-1029 showed essentially no binding to HepG2 cells.

### Example 15

### SMIP and Xceptor Blocking of Il-6 and TNF Activity in Mice

The ability of SMIP and Xceptor fusion proteins disclosed herein to block IL-6 or TNF-induced production of serum amyloid A (SAA) protein in mice was examined as described below. SAA is one of the major acute-phase proteins in humans and mice. Prolonged elevation of plasma SAA levels is found in chronic inflammation and leads to amyloidosis which affects the liver, kidney and spleen (Rienhoff et al., (1990) Mol. Biol. Med. 7:287). Both IL-6 and TNF have been shown to induce SAA when administered alone (Benigni et al., (1996) Blood 87:1851; Ramadori et al., (1988) Eur. J. Immunol. 18:1259).

(a) Blocking of hyperIL-6 Activity

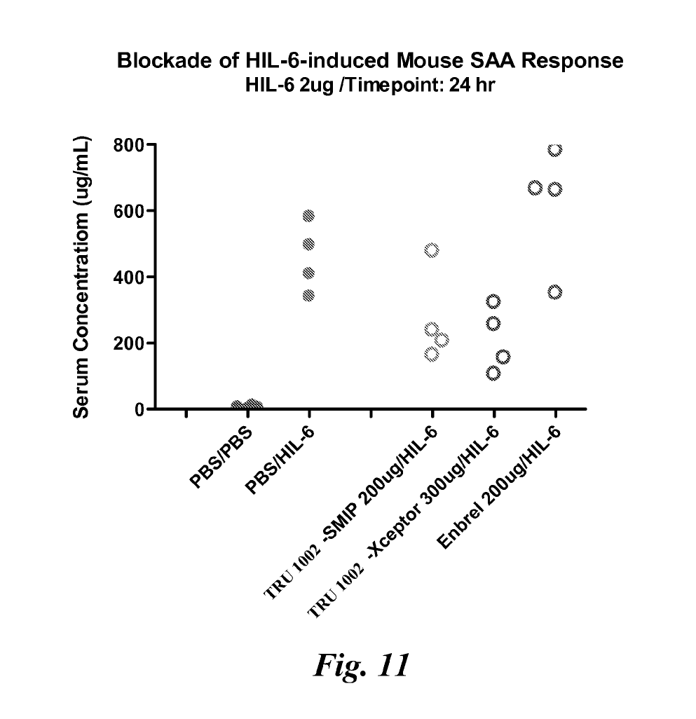

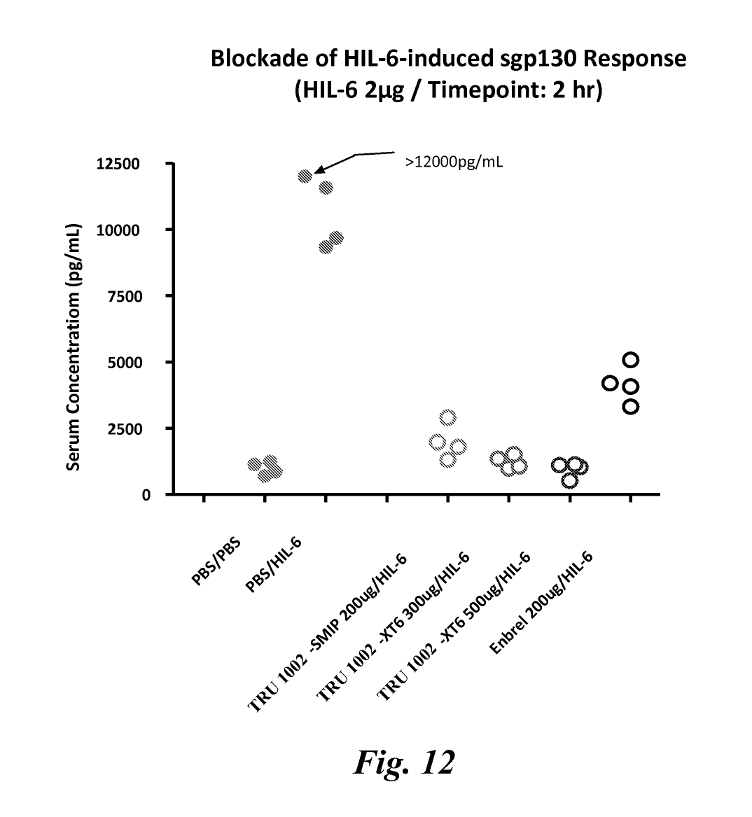

Female BALB/C mice were injected retro-orbitally with 0.2 ml PBS, or Enbrel® (200 ug), TRU(S6)-1002 (200 ug) or TRU(XT6)-1002 (300 ug or 500 ug) in PBS. One hour later, the mice were injected IP with 0.2 ml PBS or 2 μg human hyper-IL6 in PBS. Mouse serum was collected at 2 hours and 24 hours after the IP injection. The serum concentration of SAA was determined by ELISA, and concentration of sgp130 was determined by a Luminex-based mouse soluble receptor assay. As shown in FIGS. 11 and 12, TRU(S6)-1002 and TRU(XT6)-1002 blocked hyperIL6-induced expression of both sgp130 and SAA.

### (b) Blocking of TNF Activity

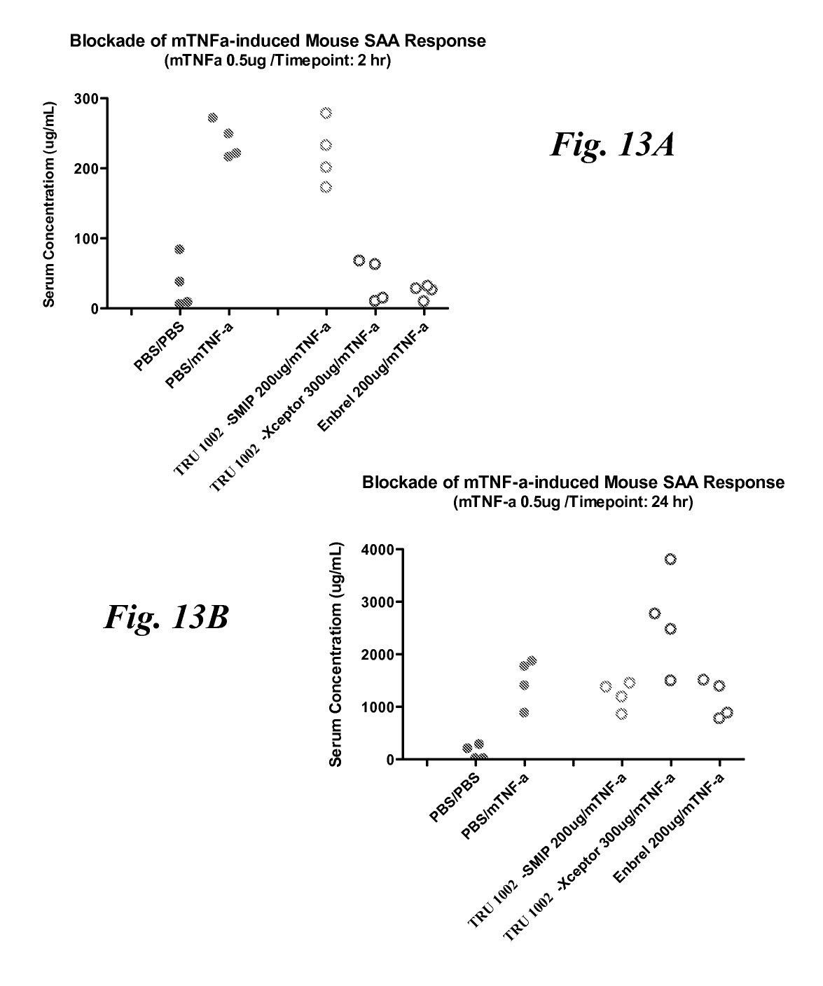

Female BALB/C mice were injected retro-orbitally with 0.2 ml PBS, or Enbrel® (200 μg), TRU(S6)-1002 (200 μg) or TRU(XT6)-1002 (300 μg) in PBS. One hour later, the mice were injected IP with 0.2 ml PBS or 0.5 ug mouse TNF-α in PBS. Mouse serum was collected at 2 hours and 24 hours after the IP injection. The serum concentration of SAA was determined by ELISA, and concentration of sgp130 was determined by a Luminex-based mouse soluble receptor assay. As shown in FIGS. 13A and B, the Xceptor TRU(XT6)-1002 blocked TNFα-induced expression of SAA, with the level of SAA observed at 2 hours post-injection being similar to that seen with Enbrel®.

### Example 16

### Xceptor Activity In Vivo

The therapeutic efficacy of Xceptor molecules described herein is examined in animal models of disease as described below.

### (a) Multiple Myeloma

The activity of Xceptor molecules is examined in at least one of two well characterized mouse models of multiple myeloma, namely the 5T2 multiple myeloma (5T2MM) model and the 5T33 multiple myeloma (5T33MM) model. In the 5T33 model, mice are treated with Xceptors from the time of injection of tumor cells (prophylactic mode). In the 5T2MM model, mice are treated from the onset of the disease (therapeutic mode). The effect of treatment on tumor development and angiogenesis is assessed in both models, with bone studies also being performed in the 5T2MM model.

The 5TMM murine model of myeloma was initially developed by Radl et al. (J. Immunol. (1979) 122:609; see also Radl et al. Am. J. Pathol. (1988) 132:593; Radl J. Immunol. Today (1990) 11:234). Its clinical characteristics resemble the human disease closely: the tumor cells are located in the bone marrow, the serum paraprotein concentration is a measure of disease development, neovascularization is increased in both the 5T2MM and 5T33MM models (Van Valckenborgh et al., Am. J. Pathol. (1988) 132:593), and in certain lines a clear osteolytic bone disease develops. The 5T2MM model includes moderate tumor growth and the development of osteolytic bone lesions. These lesions are associated with a decrease in cancellous bone volume, decreased bone mineral density and increased numbers of osteoclasts (Croucher et al., Blood (2001) 98:3534). The 5T33MM model has a more rapid tumor take and, in addition to the bone marrow, tumor cells also grow in the liver (Vanderkerken et al., Br. J. Cancer (1997) 76:451).

The 5T2 and 5T33MM models have been extensively characterized. Specific monoclonal antibodies have been raised against the idiotype of both 5T2 and 5T33MM allowing the detection, with great sensitivity, of the serum paraprotein by ELISA, and the specific staining of the tumor cells both by FACS analysis and immunostaining of histological sections (Vanderkerken et al., Br. J. Cancer (1997) 76:451). The sequence analysis of the VH gene enables the detection of cells by RT-PCR and Northern blot analysis (Zhu et al., Immunol. (1998) 93:162). The 5TMM models, which can be used for both in vitro and in vivo experiments, generate a typical MM disease and different methods are available to assess tumor load in the bone marrow, serum paraprotein concentrations, bone marrow angiogenesis (by measuring the microvessel density) and osteolytic bone lesions (by a combination of radiography, densitometry and histomorphometry). The investigation of these latter parameters allow the use of the 5TMM models in a preclinical setting and study of the growth and biology of the myeloma cells in a complete syngeneic microenvironment. Both molecules targeting the MM cells themselves and molecules targeting the bone marrow microenvironment can be studied. Specifically, while the 5T33MM model can be used to study both the microenvironment and the MM cells themselves, the 5T2MM model can also be used to study the myeloma associated bone disease.

To study the prophylactic efficacy of the Xceptor molecules disclosed herein, C57BL/KaLwRij mice are injected with 2×10⁶ 5T33 MM cells and with Xceptor on day 0. Mice are sacrificed at day 28 and tumor development is assessed by determining serum paraprotein concentration and the percentage of tumor cells on isolated bone marrow cells (determined by flow cytometry with anti-idiotype antibodies or by cytosmears). The weight of spleen and liver is determined and these organs are fixed in 4% formaldehyde for further analysis. Bone samples are fixed for further processing including CD31 immunostaining on paraffin sections and quantification of microvessel density.

To study the therapeutic efficacy of the Xceptor molecules disclosed herein, mice are injected with 5T2MM cells on day 0, and Xceptor is administered following the onset of disease, as determined by the presence of detectable levels of serum paraprotein. Mice are sacrificed approximately five weeks following administration of Xceptor, and tumor development is assessed as described above for the prophylactic study. In addition, bone analysis is performed using X-rays to determine the number of bone lesions and trabecular bone area, and TRAP staining to assess the number of osteoclasts.

### (b) Rheumatoid Arthritis

The therapeutic efficacy of the Xceptor molecules disclosed herein is examined in at least one of two murine models of rheumatoid arthritis (RA), namely the collagen induced arthritis (CIA) and glucose-6-phosphate isomerase (G6PI) models. Each of these models has been shown by others to be useful for predicting efficacy of certain classes of therapeutic drugs in RA (see Holmdahl (2000) Arthritis Res. 2:169; Holmdahl (2006) Immunol. Lett. 103:86; Holmdahl (2007) Methods Mol. Med. 136:185; McDevitt (2000) Arthritis Res. 2:85; Kamradt and Schubert (2005) Arthritis Res. Ther. 7:20).

### (i) CIA Model

The CIA model is the best characterized mouse model of arthritis in terms of its pathogenesis and immunological basis. In addition, it is the most widely used model of RA and, although not perfect for predicting the ability of drugs to inhibit disease in patients, is considered by many to be the model of choice when investigating potential new therapeutics for RA (Jirholt, J. et al. (2001) Arthritis Res. 3:87-97; Van den Berg, W. B. (2002) Curr. Rheumatol. Rep. 4:232-239; Rosloniec, E. (2003) Collagen-Induced Arthritis. In Current Protocols in Immunology, eds. Coligan et al., John Wiley & Sons, Inc, Hoboken, N.J.).

In the CIA model, arthritis is induced by immunization of male DBA/1 mice with collagen II (CII) in Complete Freund's Adjuvant (CFA). Specifically, mice are injected intradermally/subcutaneously with CII in CFA on Day-21 and boosted with CII in Incomplete Freund's Adjuvant (IFA) on Day 0. Mice develop clinical signs of arthritis within days of the boost with CII/IFA. A subset of mice (0% to 10%) immunized with CII/CFA develop signs of arthritis on or around Day 0 without a boost and are excluded from the experiments. In some CIA experiments, the boost is omitted and mice are instead treated with Xceptor or control starting 21 days after immunization with CII/CFA (i.e. the day of first treatment is Day 0).

Mice are treated with Xceptor, vehicle (PBS), or negative or positive control in a preventative and/or therapeutic regimen. Preventative treatment starts on Day 0 and continues through the peak of disease in control (untreated) mice. Therapeutic treatment starts when the majority of mice show mild signs of arthritis. Enbrel®, which has been shown to have good efficacy in both the CIA and G6PI-induced models of arthritis, is used as a positive control. Data collected in every experiment includes clinical scores and cumulative incidence of arthritis. Clinical signs of arthritis in the CIA model are scored using a scale from 0 to 4 as shown in Table 4 below:

TABLE 4

| Score | Observations |
|---|---|
| 0 | No apparent swelling or redness |
| 1 | Swelling/redness in one to three digits |
| 2 | Redness and/or swelling in more than three digits, mild swelling |
| | extending into the paw, swollen or red ankle, or mild |
| | swelling/redness of forepaw |
| 3 | Swollen paw with mild to moderate redness |
| 4 | Extreme redness and swelling in entire paw |

### (ii) G6PI Model

In the G6PI model, arthritis is induced by immunization of DBA/1 mice with G6PI in adjuvant (Kamradt, T. and D. Schubert (2005) Arthritis Res. Ther. 7:20-28; Schubert, D. et al. (2004) J. Immunol. 172:4503-4509; Bockermann, R. et al. (2005) Arthritis Res. Ther. 7:R1316-1324; Iwanami, K. et al. (2008) Arthritis Rheum. 58:754-763; Matsumoto, I. et al. (2008) Arthritis Res. Ther. 10:R66). G6PI is an enzyme present in virtually all cells in the body and it is not known why immunization induces a joint specific disease. A number of agents, such as CTLA4-Ig, TNF antagonists (e.g. Enbrel®) and anti-IL6 receptor monoclonal antibody, have been shown to inhibit development of arthritis in the G6PI model.

Male DBA/1 mice are immunized with G6PI in Complete Freund's Adjuvant (CFA) in order to induce arthritis. Specifically, mice are injected intradermally/subcutaneously with G6PI in CFA on Day 0 and develop clinical signs of arthritis within days of the immunization. As with the CIA model discussed above, mice are treated with Xceptor, vehicle (PBS), or negative or positive control in a preventative and/or therapeutic regimen. Preventative treatment starts on Day 0 and continues through the peak of disease in control mice. Therapeutic treatment starts when the majority of mice show mild signs of arthritis. Enbrel®, which has been shown to have good efficacy in both the CIA and G6PI-induced models of arthritis, is used as a positive control. Data collected in every experiment includes clinical scores and cumulative incidence of arthritis. Clinical signs of arthritis in the G6PI model are scored using a scale similar to that employed for the CIA model.

### (c) Polycystic Kidney Disease

The efficacy of an xceptor fusion protein (preferably containing a TNF antagonist and a TGFβ antagonist, as disclosed herein) in the treatment of polycystic kidney disease is tested in murine models as described in Gattone et al., Nat. Med. (2003) 9:1323-6; Tones et al. Nat. Med. (2004) 10:363-4; Wang et al. J. Am. Soc. Nephrol. (2005) 16:846-851; and Wilson (2008) Curr. Top. Dev. Biol. 84:311-50.

SEQ ID NOS:1-1255 are set out in the attached Sequence Listing. The codes for nucleotide sequences used in the attached Sequence Listing, including the symbol “n,” conform to WIPO Standard ST.25 (1998), Appendix 2, Table 1.

1. A multi-specific fusion protein comprising a structure from amino terminus to carboxy terminus selected from the group consisting of:
(a) BD-ID-ED;
(b) ED-ID-BD; and
(c) ED1-ID-ED2
wherein:
ED is a TGFβ antagonist and ED1 and ED2 are different antagonists wherein ED1 or ED2 is a TGFβ antagonist;
ID is an intervening domain; and
BD is a binding domain of a TNF antagonist, an IL6 antagonist, an IL10 antagonist, a VEGF antagonist, an HGF antagonist, an IGF antagonist, or a GITR agonist.

2. The multi-specific fusion protein of claim 1, wherein the BD is an immunoglobulin variable binding domain.

3. The multi-specific fusion protein of claim 1, wherein the ED is a receptor ligand binding domain.

4. The multi-specific fusion protein of claim 1, wherein the intervening domain has the following structure:
-L1-CH2CH3-,
wherein:
L1 is an immunoglobulin hinge linker, and
—CH2CH3- is the CH2CH3 region of an IgG1 Fc domain.

5. The multi-specific fusion protein of claim 1, wherein the BD is connected to the intervening domain by a first linker and the ED is connected to the intervening domain by a second linker and wherein the first and second linkers are the same or different.

6. The multi-specific fusion protein of claim 5, wherein the first and second linkers are selected from the group consisting of SEQ ID NO:497-604 and 1223-122.

7. The multi-specific fusion protein of claim 1, comprising an amino acid sequence which is selected from the group consisting of SEQ ID NOS:735-742.

8. A composition comprising the multi-specific fusion protein of claim and a pharmaceutically acceptable carrier, diluent, or excipient.

9. The composition of claim 8 wherein the multi-specific fusion protein exists as a dimer or a multimer in the composition.

10. A polynucleotide encoding the multi-specific fusion protein of claim 1.

11. An expression vector comprising the polynucleotide according to claim 10, which is operably linked to an expression control sequence.

12. A host cell comprising the expression vector according to claim 11.

13. A method for treating a subject with a malignant condition comprising administering to a subject in need thereof a therapeutically effective amount of the multi-specific fusion protein of claim 1.

14. The method of claim 13 wherein the malignant condition is selected from the group consisting of breast cancer, renal cell carcinoma, melanoma and prostate cancer.

15. The multi-specific fusion protein of claim 4, wherein said immunoglobulin hinge linker is an IgG1 hinge having the first cysteine substituted with a different amino acid.

16. The multi-specific fusion protein of claim 4, wherein said CH2CH3 region of an IgG1 Fc domain is mutated to eliminate FcγRI-III interaction while retaining FcRn interaction.

17. The multi-specific fusion protein of claim 6, wherein the first linker is SEQ ID NO: 576 and the second linker is SEQ ID NO: 1223.

18. A method of producing a multi-specific fusion protein comprising culturing the host cell of claim 12 in a medium and expressing the protein.
